(19) United States
(12) Patent Application Publication
(10) Pub. No.: US 2025/0127533 A1
(43) Pub. Date: Apr. 24, 2025

(54) DENTAL TOOLS FOR REMOVING DENTAL AUXILIARIES FROM TEETH

(71) Applicant: Align Technology, Inc., San Jose, CA (US)

(72) Inventors: Laura Natania Nicole Marina Lee, Knightdale, NC (US); Chang Xu, Milpitas, CA (US); Mitra Derakhshan, Herndon, VA (US); Kangning Su, Pullman, WA (US); Jonathan Biderman, Roslyn, NY (US); Jessica H. Schiltz, San Jose, CA (US); Ankit Shukla, San Jose, CA (US); Suemi Gonzales, San Jose, CA (US); Eric Yau, Santa Clara, CA (US); Crystal Tjhia, Sunnyvale, CA (US); Palak Mittal, Newark, CA (US); Yuxiang Wang, Newark, CA (US); Jivtesh Khurana, San Francisco, CA (US); Jun Sato, San Jose, CA (US); Srinivas Kaza, Mountain View, CA (US)

(21) Appl. No.: 18/919,166

(22) Filed: Oct. 17, 2024

Related U.S. Application Data
(60) Provisional application No. 63/591,430, filed on Oct. 18, 2023.
(60) Provisional application No. 63/602,278, filed on Nov. 22, 2023.

(51) Int. Cl. A61B 17/24

(52) U.S. Cl. CPC A61B 17/24

(57) ABSTRACT

Methods, devices, and systems for placing dental auxiliaries on teeth and/or removing dental auxiliaries from teeth are provided. In some embodiments, a device for removing a dental auxiliary from a tooth includes: a first elongate member having a proximal end and a distal end; a second elongate member having a proximal end and a distal end, where the first and second elongate members are pivotally coupled to each other; a scraper at the distal end of the second elongate member, where the scraper is configured to apply force to the dental auxiliary to remove the dental auxiliary from the tooth; and a pair of sidewalls configured to capture debris resulting from the removal of the dental auxiliary from the tooth.

### CROSS-REFERENCE TO RELATED APPLICATION(S)

The present application claims the benefit of priority to U.S. Provisional Application No. 63/591,430 filed Oct. 18, 2023, and U.S. Provisional Application No. 63/602,278, filed Nov. 22, 2023, each of which is incorporated by reference herein in its entirety.

### TECHNICAL FIELD

The present technology generally relates to dental and orthodontic treatment procedures, and in particular, to dental auxiliary tools and positioners and associated methods.

### BACKGROUND

Dental appliances are used to treat various dental conditions, such as dental malocclusions, jaw dysfunction/misalignment, functional and/or aesthetic conditions, endodontic conditions, and others. For example, a patient's teeth can be repositioned using a series of dental appliances that are placed successively on the teeth to provide controlled forces to gradually move the teeth toward a desired arrangement. Some treatment procedures use one or more attachments that are bonded to the patient's teeth at specific locations to interact with a dental appliance to impart repositioning forces on the teeth.

Dental auxiliaries (such as dental attachments, buttons, brackets, etc.) that may be used with dental appliances may be provided preformed or precured, including by one or more direct fabrication techniques, and may be used as part of a dental treatment. For example, a prescribed treatment may include the application of such dental auxiliaries as part of a positioner assembly that fits over the patient's teeth to assist in proper positioning of the dental auxiliary. In general, the dental auxiliaries and positioner assembly may be formed together, e.g., using an additive manufacturing or 3D printing technology. Shapes that are designed to optimize forces or retention during treatment of malocclusion can be controlled and precise. These dental auxiliaries may be bonded to the patient's teeth using the positioner assembly to locate the dental auxiliaries at the desired sites on the teeth. Once the positioner assembly is seated against the patient's dentition in a specified manner, the dental auxiliaries may be affixed to the dentition using standard orthodontic practices, e.g., a photocurable substance may be applied in a noncured, often flowable state, and a cure light may be used to solidify the material to the tooth surface.

In general, bonding of such dental auxiliaries to the patient's teeth, particularly bonding of direct fabricated (e.g., 3D printed) shapes, may benefit from the application of pressure or force while bonding. Unfortunately, applying pressure and holding a cure light can be unwieldy. Moreover, existing dental tools cannot fully meet the functional requirements for effectively bonding a dental auxiliary to a tooth, such as controlling the pressure applied against the dental auxiliary to the tooth during curing, which might compromise bonding accuracy. Existing dental tools may also lack features for separating the dental auxiliary from the positioner assembly after bonding or removing a bonded dental auxiliary from a tooth in a controlled manner without creating debris, which may increase choking risks. Thus, there is a need for apparatuses, e.g., devices and systems, including tools, to address these problems.

Moreover, it may be challenging to accurately place dental auxiliaries on teeth that are proximate to missing, unerupted, or erupting teeth. For instance, a tooth that is adjacent to a missing or unerupted tooth may tip toward the space left by the missing or unerupted tooth over time. Moreover, tooth eruption may affect the positions of neighboring teeth. In such situations, a positioner assembly that was designed based on a previous arrangement of the patient's teeth may no longer fit properly on the teeth by the time the positioner assembly is manufactured and delivered to the clinician.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1A is a perspective view of a patient's teeth with dental auxiliaries, in accordance with embodiments of the present technology.

FIG. 1B is a perspective view of a dental auxiliary positioner on the patient's teeth of FIG. 1A, in accordance with embodiments of the present technology.

FIGS. 1C-IF illustrate examples of prefabricated dental auxiliaries (in this example, dental attachments for engaging a dental appliance, such as an aligner, retainer, palatal expander, etc.), in accordance with embodiments of the present technology. FIG. 1C shows an example of a positioner assembly including multiple dental auxiliaries. FIG. 1D illustrates attachment of dental auxiliaries to a patient's teeth. FIG. 1E is an enlarged view of a dental auxiliary frangibly coupled to a frame of a positioner assembly by a plurality of frame supports. FIG. 1F is another example of a positioner assembly including a plurality of dental auxiliaries.

FIGS. 2A and 2B schematically illustrate an example of an apparatus for bonding dental auxiliaries to a tooth, in accordance with embodiments of the present technology. FIG. 2A shows the apparatus in a first configuration, in which the engager arms are disengaged, and FIG. 2B shows the apparatus in a second configuration, in which the engager arms are engaged.

FIGS. 3A and 3B schematically illustrate an example of an apparatus for bonding dental auxiliaries to a tooth, in accordance with embodiments of the present technology.

FIG. 4A schematically illustrates a distal end region of an apparatus for bonding dental auxiliaries to a tooth, showing engagement of the device with a dental auxiliary and a frame to which the dental auxiliary is attached, in accordance with embodiments of the present technology.

FIG. 4B schematically illustrates an example of a distal end region of an apparatus for bonding dental auxiliaries to a tooth, in accordance with embodiments of the present technology.

FIG. 5 schematically illustrates an example of a handle, including an actuating control for actuating movement of an engager and/or engager arm of an apparatus for bonding dental auxiliaries to a tooth, in accordance with embodiments of the present technology.

FIG. 6 schematically illustrates an example of an engager arm of an apparatus for bonding dental auxiliaries to a tooth, in accordance with embodiments of the present technology.

FIGS. 7A and 7B illustrate another example of an apparatus for bonding dental auxiliaries to a tooth, in accordance with embodiments of the present technology. FIG. 7B shows an enlarged view of the distal end region of one example of an apparatus for bonding dental auxiliaries to a tooth, similar to that shown in FIG. 7A.

FIG. 8 illustrates one example of a distal end region of an apparatus for bonding dental auxiliaries to a tooth including a rotatable joint, in accordance with embodiments of the present technology.

FIGS. 9A-9C schematically illustrate an example of an adapter for a light curing device for use with a dental auxiliary, in accordance with embodiments of the present technology. FIG. 9A schematically illustrates an example of a light curing device. FIG. 9B schematically illustrates an adapter for a light curing device. FIG. 9C schematically illustrates the adapter of FIG. 9B coupled to the light curing device of FIG. 9A.

FIG. 10 illustrates an example of a method of bonding a dental auxiliary to a tooth, in accordance with embodiments of the present technology.

FIG. 11 schematically illustrates an example of an apparatus for removal of a dental auxiliary from a tooth, in accordance with embodiments of the present technology.

FIG. 12 schematically illustrates an example of an apparatus for removal of a dental auxiliary from a tooth, in accordance with embodiments of the present technology. In FIG. 12 the apparatus includes a central suction inlet and a shield around an occlusal gripper region.

FIG. 13 schematically illustrates an example of an apparatus for removal of a dental auxiliary from a tooth, in accordance with embodiments of the present technology. In FIG. 13 the apparatus includes a suction inlet adjacent to a contact region (e.g., occlusal gripper) for the dental auxiliary.

FIG. 14 illustrates one example of a close-up of a distal end region of an apparatus for removal of a dental auxiliary from a tooth, in accordance with embodiments of the present technology.

FIG. 15 schematically illustrates an example of an apparatus for removal of a dental auxiliary from a tooth, in accordance with embodiments of the present technology. In FIG. 15 the apparatus includes a suction inlet in a contact region (e.g., occlusal gripper) for the dental auxiliary.

FIGS. 16A and 16B illustrate one example of a method of removing a dental auxiliary using an apparatus as described herein, in accordance with embodiments of the present technology.

FIGS. 17A-17C illustrate a dental auxiliary removal device, in accordance with embodiments of the present technology.

FIGS. 18A-18E illustrate a dental auxiliary removal device, in accordance with embodiments of the present technology.

FIGS. 19A-19D illustrate a dental auxiliary removal device, in accordance with embodiments of the present technology.

FIGS. 20A and 20B illustrate a dental auxiliary removal device with a sleeve to capture debris, in accordance with embodiments of the present technology.

FIG. 20C illustrates a dental auxiliary removal device configured in accordance with embodiments of the present technology.

FIG. 20D illustrates a combined dental auxiliary placement and removal device, in accordance with embodiments of the present technology.

FIG. 21A is a side view of a dental arch including a space due to one or more missing and/or unerupted teeth.

FIG. 21B is a side view of a dental arch including a space due to an erupting tooth.

FIG. 22 is a schematic block diagram illustrating a workflow for placing a dental auxiliary on a tooth, in accordance with embodiments of the present technology.

FIG. 23 is a flow diagram illustrating a method for placing a dental auxiliary on a tooth, in accordance with embodiments of the present technology.

FIG. 24A illustrates a user interface for displaying a comparison between a first tooth arrangement and a second tooth arrangement of a patient's teeth, in accordance with embodiments of the present technology.

FIG. 24B illustrates a digital dental representation for displaying a comparison between a first tooth arrangement and a second tooth arrangement of a patient's teeth, in accordance with embodiments of the present technology.

FIG. 25 is a perspective view of a device for measuring movement of a patient's teeth, in accordance with embodiments of the present technology.

FIG. 26 is a perspective view of a device for measuring movement of a patient's teeth, in accordance with embodiments of the present technology.

FIG. 27A is a side view of an adjustable dental auxiliary positioner, in accordance with embodiments of the present technology.

FIG. 27B is a partially schematic side view of an adjustable dental auxiliary positioner, in accordance with embodiments of the present technology.

FIG. 27C is a partially schematic side view of another adjustable dental auxiliary positioner, in accordance with embodiments of the present technology.

FIG. 27D is a partially schematic side view of another adjustable dental auxiliary positioner, in accordance with embodiments of the present technology.

FIG. 28A is a side view of a registration element with stabilizing features, in accordance with embodiments of the present technology.

FIG. 28B is a top view of the registration element of FIG. 28A, in accordance with embodiments of the present technology.

FIG. 28C is a perspective view of another registration element with stabilizing features, in accordance with embodiments of the present technology.

FIG. 28D is a perspective view of a dental auxiliary positioner including at least one registration element with stabilizing features, in accordance with embodiments of the present technology.

FIG. 29 is a schematic block diagram illustrating a workflow for placing a dental auxiliary on a tooth, in accordance with embodiments of the present technology.

FIG. 30 is a flow diagram illustrating a method for placing a dental auxiliary on a tooth, in accordance with embodiments of the present technology.

FIG. 31A is a partially schematic top view of a dental spacing device, in accordance with embodiments of the present technology.

FIG. 31B is a partially schematic top view of another dental spacing device, in accordance with embodiments of the present technology.

FIG. 31C is a partially schematic top view of yet another dental spacing device, in accordance with embodiments of the present technology.

FIG. 32A is a partially schematic top view of a dental spacing device with a deformable spacer portion, in accordance with embodiments of the present technology.

FIG. 32B is a partially schematic top view of the dental spacing device of FIG. 32A when placed on a patient's teeth, in accordance with embodiments of the present technology.

FIG. 33 is a partially schematic top view of a dental spacing device with an adjustable spacer portion, in accordance with embodiments of the present technology.

FIG. 34A is a partially schematic top view of a dental spacing device with an aesthetic portion, in accordance with embodiments of the present technology.

FIG. 34B is a partially schematic side view of the dental spacing device of FIG. 34A, in accordance with embodiments of the present technology.

FIG. 35A illustrates a representative example of a tooth repositioning appliance configured in accordance with embodiments of the present technology.

FIG. 35B illustrates a tooth repositioning system including a plurality of appliances, in accordance with embodiments of the present technology.

FIG. 35C illustrates a method of orthodontic treatment using a plurality of appliances, in accordance with embodiments of the present technology.

FIG. 36 illustrates a method for designing an orthodontic appliance, in accordance with embodiments of the present technology.

FIG. 37 illustrates a method for digitally planning an orthodontic treatment and/or design or fabrication of an appliance, in accordance with embodiments of the present technology.

### DETAILED DESCRIPTION

The present technology relates to methods and apparatuses (e.g., devices and systems, including tools) for positioning dental auxiliaries on a patient's teeth and/or removing dental auxiliaries from a patient's teeth. As used herein, a dental auxiliary may include dental attachments, buttons, power arms, brackets, splints, distalizers, wires, or any other fixture for aiding a dental treatment. The dental auxiliary may be manufactured and/or coupled to a positioner or support assembly (e.g., posts, struts, frame, and/or a positioner assembly) for positioning and/or bonding to one or more teeth in a desired location.

An apparatus for bonding a dental auxiliary to a tooth may include a light output region at the distal end for radiating light having a wavelength that is configured to cure a light-curable dental compound (e.g., a bonding material). These apparatuses may include one or more features to assist in bonding or removing of the dental auxiliary to or from one or more teeth, and/or one or more features to assist in removing or separating one or more dental auxiliaries from a frame (or positioner assembly). In some embodiments, the apparatus may advantageously include both features to assist in orthodontic bonding or removal of one or more dental auxiliaries from the dentition, and in removing or separating the one or more dental auxiliaries from the positioning or supporting apparatus.

Features to assist in bonding of the dental auxiliary to the tooth may include one or more sensors that may detect contact and/or pressure between the dental auxiliary upon the dentition and the apparatus, and in particular, between the dental auxiliary and a contact region of the apparatus. The apparatus may provide output to the user indicating that the contact and/or applied pressure is within a target range (or not within a target range) and/or is (or is not) being applied at a target angle of approach. The apparatus may include hardware, software and/or firmware for determining if the contact and/or pressure applied to the dental auxiliary is within a target range, is applied from a target direction, and/or is being applied for a target duration while applying the method of bonding or removal (e.g., mechanical, thermal, or chemical inputs) with the apparatus.

Features to assist in removing or separating the dental auxiliary from a positioning or supporting positioner assembly may include engagers and/or engager arms. An engager may be a jaw, hook, clasp, clip, snare, hasp, etc., that contacts one or more positioner assemblies and at least transiently connects to the positioner assembly. An engager may be at the end of an engager arm. An engager arm may movably extend from a distal end region of the apparatus and may be actuated to engage with and/or to disengage the dental auxiliary from a frame. The apparatus may include an engager without an engager arm, or may include an engager arm without an engager. For example, the apparatus may include an engager arm that extends to contact the frame and rotate, vibrate, or otherwise push against the frame to separate one more dental auxiliary from the frame at specific regions of contact.

In some embodiments, a device for removing a dental auxiliary from a tooth is provided. The device can include a first elongate member having a proximal end and a distal end; a second elongate member having a proximal end and a distal end, where the first and second elongate members are pivotally coupled to each other; a scraper at the distal end of the second elongate member, where the scraper is configured to apply force to the dental auxiliary to remove the dental auxiliary from the tooth; and a pair of sidewalls configured to capture debris resulting from the removal of the dental auxiliary from the tooth.

In some embodiments, a method for placing a dental auxiliary on a tooth includes providing a dental auxiliary positioner having an auxiliary to be mounted on a tooth of a patient. The dental auxiliary positioner can be provided in a first configuration corresponding to a first tooth arrangement of the patient's teeth. For instance, the dental auxiliary positioner can be designed based on scan data of the first tooth arrangement of the patient's teeth, and thus can be configured to fit on the patient's teeth in the first tooth arrangement. The method can include determining a second tooth arrangement of the patient's teeth, where the second tooth arrangement corresponds to the patient's teeth at a time subsequent to the first tooth arrangement. A second position of the tooth in the second tooth arrangement may be different from a first position of the tooth in the first tooth arrangement, e.g., the tooth may move over time due to being proximate to a missing, unerupted, or erupting tooth. The method can further include adjusting the dental auxiliary positioner to a second configuration to accommodate the difference between the first and second positions of the tooth, then placing the auxiliary on the tooth using the adjusted dental auxiliary positioner.

As another example, a method for placing a dental auxiliary on a tooth can include obtaining, at a first time, data indicative of a tooth arrangement of a patient's teeth (e.g., scan data). The patient's teeth may include a tooth proximate to a space associated with a missing, unerupted, or erupting tooth. The method can also include positioning a dental spacing device into or proximate to the space proximate to the tooth, where the dental spacing device is configured to inhibit movement of the tooth relative to the space. For instance, the dental spacing device can obstruct movement of the tooth toward and/or away from the space, thereby maintaining the position of the tooth. The method can further include placing, at a second time after the first time, an auxiliary on the tooth using a dental auxiliary positioner. The dental auxiliary positioner can have a configuration based on the data of the tooth arrangement of the patient's teeth at the first time.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” “lower,” “left,” “right,” etc., can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Dental Auxiliaries and Positioners

The present technology provides systems, methods, and devices that may be used in combination with dental auxiliaries (e.g., dental attachments, buttons, power arms, brackets, splints, distalizers, wires) and/or with positioners for placing dental auxiliaries on a patient's teeth (which may also be referred to herein as “dental auxiliary positioners” or “positioner assemblies.”)

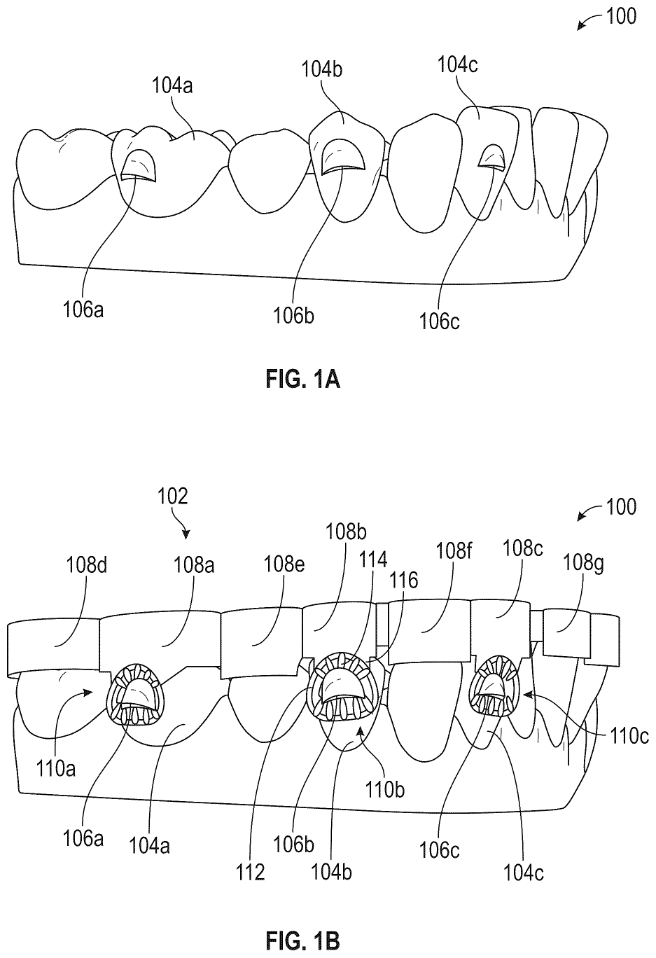

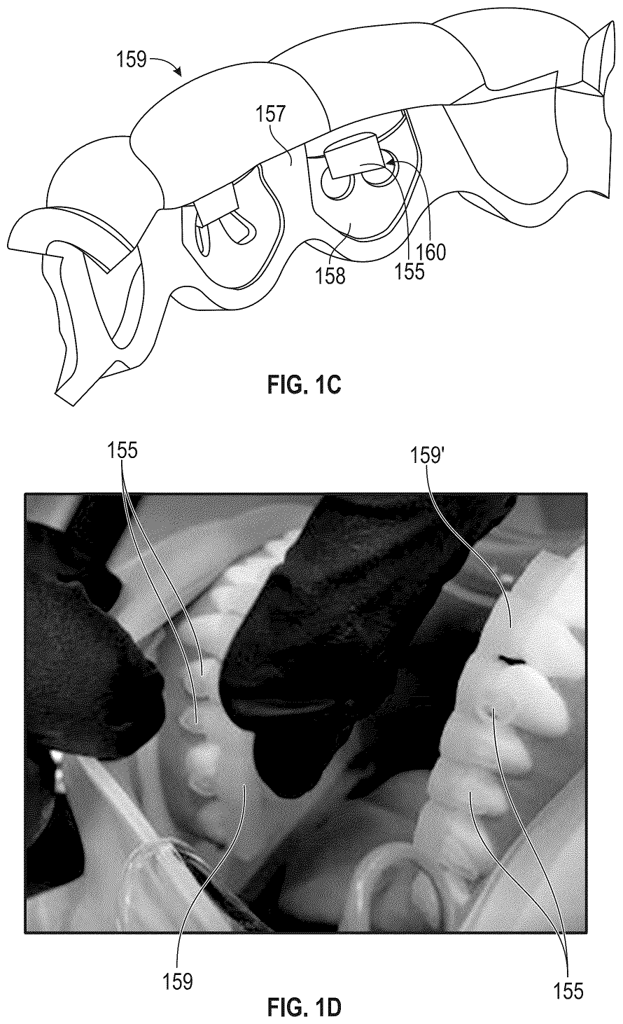

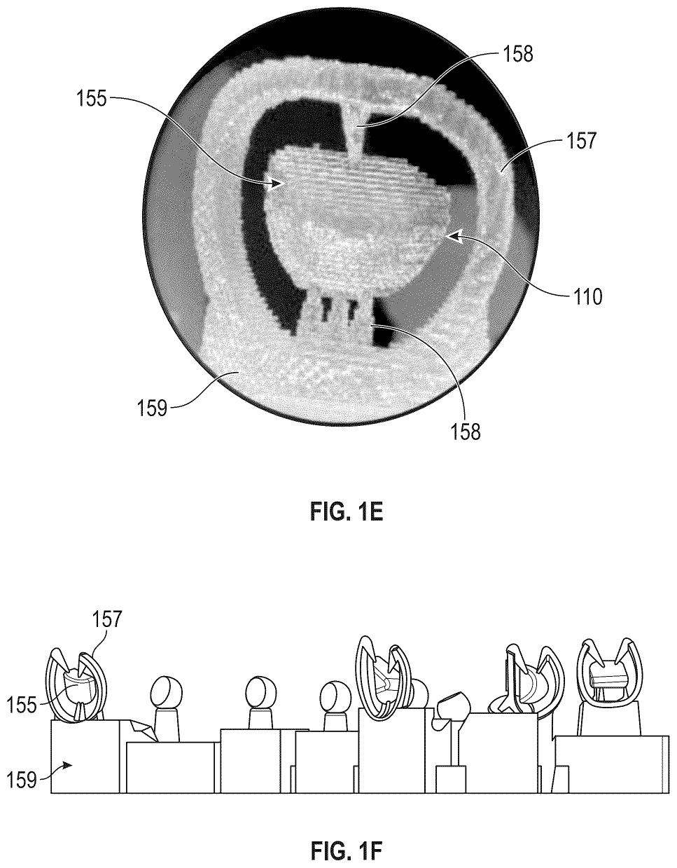

FIG. 1A is a perspective view of a patient's teeth 100, and FIG. 1B is a perspective view of a dental auxiliary positioner (“positioner 102”) on the teeth 100, in accordance with embodiments of the present technology. Referring first to FIG. 1A, the teeth 100 can include one or more teeth (e.g., a first tooth 104a, a second tooth 104b, and a third tooth 104c-collectively, “teeth 104”) having respective auxiliaries mounted thereon (e.g., a first auxiliary 106a, a second auxiliary 106b, and a third auxiliary 106c-collectively, “auxiliaries 106”). In some embodiments, the auxiliaries 106 are bonded to the teeth 104 using an adhesive, such as a curable adhesive (e.g., a UV-curable adhesive). An “auxiliary” or “dental auxiliary” can be any object that is affixed to one or more teeth and that is designed to engage a dental appliance (e.g., an aligner, palatal expander, retainer, mouth guard) to facilitate an orthodontic treatment or to maintain/protect teeth, such as dental attachments, buttons, power arms, brackets, splints, distalizers, wires, etc. In the illustrated embodiment, for example, the auxiliaries 106 are attachments that engage respective portions of a dental appliance (not shown) to produce forces for repositioning the teeth 104. Auxiliaries may be placed on the lingual, buccal, and/or occlusal surfaces of one or more teeth. As used herein, the term “occlusal auxiliary” includes occlusal blocks, bite ramps, attachments, or any other suitable projecting element placed on an occlusal side of a tooth for the purpose of, e.g., spacing apart or disoccluding teeth of the upper and lower jaw. For example, an occlusal auxiliary may be an occlusal block placed along one or more posterior teeth (and/or anterior teeth) so as to disocclude teeth for allowing jaw movement (e.g., anterior or posterior jaw movement) to correct, e.g., an underbite, an overbite, a crossbite, etc.

Referring next to FIG. 1B, the positioner 102 can be used to place the auxiliaries 106 at predetermined locations on the teeth 104 so that the auxiliaries 106 engage properly with the dental appliance. In some embodiments, the positioner 102 includes a plurality of registration elements 108a-108g (collectively, “registration elements 108”) that are configured to receive a subset of the teeth 100 (e.g., a quadrant) or all of the teeth 100. A registration element 108 may receive a tooth by, for example, receiving and/or extending over only a portion of the tooth, such as the crown of the tooth, a portion of the tooth proximate to the crown, a buccal portion of an occlusal surface of the tooth, etc. The registration elements 108 can be connected to each other via rigid or flexible couplings to form a unitary appliance body. Each registration element 108 is configured to receive and engage a corresponding tooth to retain the positioner 102 in a specified spatial configuration with respect to the teeth 100. For instance, each registration element 108 can include a cavity formed therein to define an inner surface that engages a corresponding surface of the received tooth (e.g., the occlusal, buccal, and/or lingual surface of the tooth). The topography of the inner surface can be complementary to the topography of the corresponding surface of the tooth.

The positioner 102 includes one or more auxiliary supports (e.g., a first auxiliary support 110a, a second auxiliary support 110b, and a third auxiliary support 110c-collectively, “auxiliary supports 110”) that are each configured to position a corresponding auxiliary 106 at a predetermined location against or proximate to a surface of a tooth 104 when the positioner 102 is placed on the patient's teeth 100. In the illustrated embodiment, each auxiliary support 110 is coupled to a corresponding registration element 108 that receives the tooth 104 to which the auxiliary 106 is to be mounted, such that when the registration element 108 engages the tooth 104, the auxiliary 106 is positioned at a predetermined location on the tooth 104. Optionally, the registration element 108 can be coupled to a retention support (not shown) that engages another portion of the received tooth 104 (e.g., the lingual surface when the auxiliary is to be placed on the buccal side, the buccal surface when the auxiliary is to be placed on the lingual side, an interproximal region, and/or an undercut region) to produce a force that biases or maintains the auxiliary support 110 toward/against the tooth 104 to stabilize the positioner 102 and/or to facilitate positioning of the auxiliary 106 against the surface of the tooth 104.

In the illustrated embodiment, the auxiliary support 110 includes a frame 112 that extends partially or entirely around a perimeter of the auxiliary 106, e.g., to protect/stabilize the auxiliary 106 during manufacturing, shipment, and/or handling. The frame 112 can be coupled directly to the registration element 108, or can be coupled indirectly via a bridge 116 (also known as a “frame belt”). The bridge 116 can be flexible to permit some movement of the frame 112 and auxiliary 106 relative to the corresponding registration element 108, or can be rigid so that the frame 112 and auxiliary 106 are maintained in a fixed spatial relationship with respect to the corresponding registration element 108.

The auxiliary 106 can be coupled to the frame 112 via a plurality of struts 114. Each strut 114 can be an elongate member including a first end connected to the frame 112 and a second end connected to the auxiliary 106. The struts 114 can be distributed around the perimeter of the auxiliary 106. In the illustrated embodiment, for example, the auxiliary support 110 includes a first set of struts 114 at a first (e.g., gingival) side of the auxiliary 106, and a second set of struts 114 at a second (e.g., occlusal) side of the auxiliary 106. In some embodiments, the struts 114 are breakable components that are fractured to release the auxiliary 106 from the positioner 102 after the auxiliary 106 has been bonded to the tooth 104, thus allowing the positioner 102 to be removed while the bonded auxiliary 106 remains in place on the tooth 104. Optionally, the frame 112 can be omitted, such that the struts 114 are connected directly to the bridge 116 and/or directly to the registration element 108.

The auxiliaries 106 can be prefabricated components that are manufactured together with the auxiliary supports 110 and registration elements 108, such that each auxiliary 106 is integrally formed with its respective frame 112 and struts 114. In some embodiments, the entire positioner 102, including the auxiliaries 106, auxiliary supports 110, and registration elements 108, is fabricated in a single manufacturing operation (e.g., via a single additive manufacturing process). Alternatively, some portions of the positioner 102 can be discrete components that are fabricated separately (e.g., via separate additive manufacturing processes) and subsequently assembled to form the positioner 102, e.g., the auxiliary supports 110 and auxiliaries 106 can be fabricated in a different manufacturing operation than the registration elements 108.

FIGS. 1C-IF illustrate examples of dental auxiliaries and positioner assemblies. In FIG. 1C, a positioner assembly 159 is shown including multiple frames 157, each frame 157 being attached to a dental auxiliary 155 via respective frame supports 158 (e.g., struts) that position the dental auxiliary 155 in a desired location. In some embodiments, the positioner assembly may be “frameless” and may support the dental auxiliary 155 in the desired location without surrounding it. The positioner assembly 159 may be applied over a portion of the teeth and may include one or more cavities that fit over one or more associated teeth of a patient. The positioner assembly 159 is configured to position the dental auxiliaries 155 (shown as attachments in FIGS. 1C-IF) against the teeth to which they are to be bonded. The frame supports 158 for the dental auxiliary 155 are configured to be severed after the dental auxiliary 155 has been positioned and bonded to a desired location on a tooth of a patient.

In the example shown in FIG. 1C, the dental auxiliary 155 is connected to the frame 157 on the positioner assembly 159 by one or more frame supports 158, and the space between the dental auxiliary 155 and the frame 157 may include gaps or apertures 160. Although FIG. 1C illustrates three frame supports 158 connecting to the dental auxiliary 155 at three points, the disclosure contemplates any suitable number of frame supports 158, including a single frame support 158 connecting to the dental auxiliary 155 at one point. In some embodiments, an engager and/or engager arms of another apparatus may engage with either the frame 157 or the dental auxiliary 155 through the gaps/apertures 160 and/or through the frame supports 158, as discussed further herein, e.g., in Section II below.

FIG. 1D illustrates one example of a first positioner assembly 159 and a second positioner assembly 159′ that each include a plurality of dental auxiliaries 155 that are attached to the frame. In this example, the first positioner assembly 159 is configured to be worn on the patient's upper jaw while the second positioner assembly 159′ is configured to be applied to the patient's lower jaw. In some embodiments, each positioner assembly may only cover a subset of teeth of the patient's upper/lower jaw. For example, a positioner assembly may be configured to cover a quadrant (or other suitable segment) of a patient's jaw. In this example, four quadrants may be formed for a patient (e.g., two quadrants for the upper jaw and two quadrants for the lower jaw). In other embodiments, the positioner assembly may be formed in any other suitable number of segments (e.g., three segments per jaw).

FIG. 1E provides additional detail on the dental auxiliary 155, which is attached to the positioner assembly 159 by a plurality of frame supports 158 that extend between the dental auxiliary and the frame 157. The positioner assembly 159 may include a plurality of different frames that each connect to a dental auxiliary.

FIG. 1F illustrates an alternative example of a positioner assembly 159 including a plurality of dental auxiliaries 155 that are each connected to a frame 157. The connection between an auxiliary and its respective frame supports may be frangible (e.g., the frame supports may be formed to be tapered and/or thin so as to allow easy severing, the connection point may include a perforation, the frame supports may be made of a brittle material, etc.). As described herein, this frangible connection may be broken in a controlled manner by the actuation of one or more engager arms and/or engagers using the device for bonding dental auxiliaries.

### II. Placement and Removal Tools for Dental Auxiliaries

Described herein are apparatuses (devices and systems, including tools) and methods for attaching dental auxiliaries to a patient's teeth, as well as apparatuses and method for removing dental auxiliaries. Dental auxiliaries may include dental attachments, buttons, power arms, brackets, lingual wires, or any other fixture that is fixed to one or more teeth for engaging with a dental appliance (e.g., a clear aligner, a palatal expander, a mouth guard, a retainer) for aiding an orthodontic treatment. In some embodiments, the dental auxiliaries may be manufactured and/or coupled to a frame and/or a positioner assembly for placement and/or bonding to one or more teeth. In general, dental auxiliaries may be preformed features that are bonded to a patient's teeth and may benefit from attaching while applying an amount of pressure that is sufficient to allow a bonding material (e.g., a light-curable dental compound) to fully harden, and/or applying pressure from an appropriate direction so that the bonding material bonds the dental auxiliary to the teeth in a desired orientation and/or position.

When removing dental auxiliaries from the frames that hold them, particularly small dental auxiliaries, existing dental tools (e.g., pliers) do not prevent features (or fragments of features, break-away attachments, frame, etc.) from falling into the patient's mouth. The same issue may occur when removing the features from the teeth following treatment. In addition, since the dental auxiliary is coupled to a support feature (e.g., struts, posts, frames, positioner assembly, a jig, etc.) the dental auxiliary may need to be separated from the supporting positioner without disrupting the placement of the dental auxiliary on the tooth, and without releasing fragments or pieces of the frame. In some cases, it may be difficult to detach a dental auxiliary from a frame, as the dental auxiliary may be irregularly shaped and/or small, or access to the region once the dental auxiliary is placed may be impeded by the patient's dentition. In addition, if the dental auxiliary is not held against the tooth with sufficient pressure when bonding, the resulting bond may be weak, and may fail prematurely. The apparatuses, including devices for bonding dental auxiliaries, described herein may assist the user (e.g., orthodontist, dentist, trained staff, technicians, device user) in holding the dental auxiliary against the tooth and may assist in removing the dental auxiliary from the frame without disrupting the placement of the dental auxiliary. In general, these apparatuses may engage with both the dental auxiliary and the frame to which the dental auxiliary is coupled, and may assist or control detachment of the dental auxiliary from the frame while preventing the frame, or pieces of the frame, from being released in an uncontrolled manner. For example, any of these apparatuses may include one or more engager arms configured to engage with a frame or regions of a frame (e.g., frame supports, positioner assembly), which may assist in either separating the dental auxiliary from the frame and/or collection of the frame or piece of the frame once the dental auxiliary is separated from the frame. These apparatuses may additionally or alternatively be configured to assist the user in ensuring that sufficient contact and/or pressure is applied to the dental auxiliary during bonding, particularly when applying energy (e.g., light energy) to form the bond between the dental auxiliary and the tooth. These apparatuses may be configured to monitor the pressure and provide passive (e.g., indicated by an alert) or active (e.g., locking/unlocking one or more engager arms) assistance to prevent premature removal of the apparatus from the dental auxiliary when bonding the dental auxiliary to the tooth/teeth.

These apparatuses may be configured as devices (e.g., tools, including dental auxiliary bonding tools) for assisting a user in attaching a dental auxiliary to a tooth, for reliably and safely separating the dental auxiliary from the frame, and/or for collecting or removing the frame or pieces of the frame during separation. In some embodiments, the apparatus is an integrated dental auxiliary tool that includes an energy source that may be used to activate (e.g., crosslink) orthodontic bonding agent(s) for bonding the dental auxiliary to the tooth, as well as one or more engagers to which the dental auxiliary can be attached and/or for assisting in separation of the dental auxiliary from its positioner assembly following bonding. The engager may be at the end of an engager arm that may be operable (e.g., retractable, extendable, rotatable, vibratory) to help secure the frame or piece of the frame, and/or may help separate the dental auxiliary from the positioner assembly.

Alternatively, in some embodiments, the apparatus (e.g., device or tool) for bonding the dental auxiliary to the tooth or teeth may be configured to couple with an existing light applicator (e.g., cure light or cure tray) to expand the functionality of the light applicator. Any of these apparatuses may be configured to secure the dental auxiliary against the dentition until the auxiliaries are bonded to the teeth and can be separated from the positioner assembly. These apparatuses may be used manually, e.g., by a user holding the apparatus. In particular, these apparatuses may be configured for one-handed use. In some embodiments, these apparatuses may be configured for use with a robotic system. For example, once the apparatus is inserted by the user, the apparatus is self-retentive in the oral cavity and the methods of bonding and removing can occur automatically, e.g., in a sequence.

For example, described herein are devices for bonding a dental auxiliary to a tooth, including: a proximal portion (e.g., user-facing portion) and a distal portion (e.g., dental auxiliary-to-tooth interface portion). The distal portion can include one or more energy output regions configured to emit energy (e.g., light or other electromagnetic radiation) at intensities and wavelengths configured to cure a bonding material (e.g., a light-curable dental compound such as orthodontic adhesive, resin, paste, primer, cement, etc.), and one or more engager arms configured to interface with one or more frames coupled to a dental auxiliary, where the one or more frames are part of a positioner assembly.

In any of these apparatuses and methods, the energy output region(s) may include and/or may be optically connected to an energy source that may be, e.g., an LED light. The energy source (e.g., LED light), may be located at the distal portion, or it can be located more proximally, and the energy may be directed, e.g., via tubes, tunnels, pipes, fiber optics, etc., to the energy output region(s). The energy source may be a narrow-spectrum or broad-spectrum light source, and the wavelength of the light may be selected to activate the bonding material.

In some embodiments, the engager arm(s) are configured to interface with one or more frames that are coupled to the dental auxiliary. For example, the engager arm(s) may include an engager that grabs, attaches to, captures, holds, or otherwise connects with the frame(s). The engager may be a mechanical engager, such as a jaw, hook, clasp, clip, snare, hasp, etc., that contacts the frame and at least transiently attaches to it. Alternatively or additionally, the engager may be or may include an adhesive (e.g., chemical) or magnetic material that is configured to be coupled to the frame(s) or to select areas along the positioner assembly so as to secure the frame(s). In some embodiments, the frame(s) may include one or more engagement regions to interact with the engager and/or engager arm(s) in a predictable manner. The engager arm(s) may be configured to move relative to the device, and in particular, relative to the portion of the device (e.g., a dental auxiliary contact region) that contacts and/or holds the dental auxiliary, so as to separate the dental auxiliary from the frame(s) after bonding. The engager arm(s) may be configured to extend, retract, pivot, rotate, etc.

In some embodiments, any of these methods and apparatuses may include an engager arm that is configured to move against the frame(s), and in particular, against one or more frame supports securing the dental auxiliary to the frame(s). In some embodiments, the apparatus may also include a rotatable joint that is configured to cause one or more engager arms to rotate after the one or more frames have been engaged so as to cause the one or more frames to break.

In some embodiments, the apparatuses described herein may be configured for manual operation and/or for robotic (e.g., automated or semi-automated) operation. Any of these apparatuses may include one or more actuating controls configured to actuate the one or more engager arms to engage the frame from the positioner assembly. For example, an apparatus may include an actuating control that is configured to extend the one or more engager arms distally to engage with the one or more frames. In some embodiments, the actuating control may be configured to retract the one or more engager arms proximally, e.g., to disengage the frame from the positioner assembly. The actuating control may be located on a body or handle of the apparatus. In some embodiments, the actuating control may be a switch, trigger, button, slider, lever, dial, etc.

In any of these methods and apparatuses, the apparatus may be configured to retract proximally to remove the dental auxiliary from the frame. Any of these apparatuses may be configured to prevent or defer movement of the one or more engager arms and subsequent separation of the dental auxiliary from the frame until the bonding material used to secure one or more dental auxiliaries to the dentition is cured. For example, the apparatus may be configured to delay actuation of the engager arm(s) until a predetermined bonding time period has elapsed, and the predetermined bonding time period may be predetermined and/or may be set by the user. In some embodiments, the apparatus may confirm contact between the dental auxiliary and the device while applying the energy for a sufficient time to allow the bonding material to cure. This may prevent movement (e.g., retracting) of the engager arm(s) until the dental auxiliary is attached and/or bonded to the tooth, and may also allow repositioning of the dental auxiliary and/or apparatus.

Any of these apparatuses may include a dental auxiliary contact region configured to engage with the dental auxiliary. This dental auxiliary contact region may be configured to associate (e.g., attach, join, link, unite, hold, contain, fit over, mate) with some portion of the dental auxiliary. In some embodiments, the dental auxiliary contact region includes a cavity or recess that is sized and/or shaped to hold the dental auxiliary. The dental auxiliary contact region may be concave or may include one or more concavities into which the dental auxiliary may fit. The dental auxiliary contact region may include a concave surface. The dental auxiliary contact region may adhere or secure to the dental auxiliary. In some embodiments, the dental auxiliary contact region may include an adhesive or magnetic material for releasably holding the dental auxiliary. Alternatively or additionally, the dental auxiliary contact region may be configured to mate with one or more region or markers on the dental auxiliary. For example, the dental auxiliary may include a mating region for mating with a complementary region of the dental auxiliary contact region. In some embodiments, the dental auxiliary contact region may include one or more projections that releasably couple with the dental auxiliary. Alternatively or additionally, the dental auxiliary contact region may include one or more slots, channels, etc., for engaging with a projection (e.g., pin, bump, protrusion) on the dental auxiliary.

In some embodiments, the dental auxiliary contact region includes one or more sensors for detecting and/or monitoring contact between the dental auxiliary and the apparatus. In some embodiments, the dental auxiliary contact region includes one or more sensors for detecting and/or monitoring the pressure (e.g., applied force) between the dental auxiliary and the dental auxiliary contact region of the apparatus. For example, any of these apparatuses and methods may include a sensor configured to sense a pressure applied by the dental auxiliary contact region against the dental auxiliary seated within the dental auxiliary contact region. Any appropriate pressure sensor and/or contact sensor may be used. For example, the pressure sensor may include a mechanical pressure sensor (e.g., strain gauge), a piezoelectric pressure sensor, a capacitive strain gauge, a pneumatic pressure sensor, etc.

In some embodiments, the dental auxiliary contact region may be configured to transmit the energy (e.g., light or other electromagnetic) via the one or more energy output regions. For example, the contact region may include one or more outputs (e.g., transparent regions) for transmitting light from one or more energy output regions to the dental auxiliary and/or the bonding material.

Any of these apparatuses may include a handle or handle region. The handle may be configured to be manually held within a user's hand. The handle may be at the proximal portion of the apparatus. The handle may include an actuating control configured to actuate the one or more engager arms and/or the one or more engagers. In some embodiments, the handle may include a light control input configured to activate the energy output regions to radiate energy (e.g., light) to cure the bonding material (e.g., a light-curable dental compound). The light control input may be a separate control or the same control as the actuating control configured to control movement of the one or more engager arms. In some embodiments, the light control input may be a button, a switch, etc.

In some embodiments, each of the one or more engager arms may include one or more engagers at a distal end region of each of the one or more engager arms that is configured to engage with the one or more frames.

In some embodiments, devices for bonding dental auxiliaries to a tooth are provided. A device may include: an actuation body having a proximal end and a distal end; a tip region comprising one or more light output regions configured to radiate light having a wavelength that is configured to cure a light-curable dental compound; a dental auxiliary contact region configured to engage with a dental auxiliary; one or more engager arms at a distal end region of the tip region, the one or more engager arms configured to engage one or more frames coupled to the dental auxiliary, where the one or more frames are part of a positioner assembly; and an actuating control on the actuation body or a handle, where the actuating control is configured to cause the one or more engager arms to engage with the one or more frames coupled to the dental tooth auxiliary while the dental auxiliary is engaged with the dental auxiliary contact region.

In some embodiments, a device for bonding dental auxiliaries to a tooth may include a proximal portion and a distal portion, the distal portion including: one or more light output regions configured to radiate light having a wavelength configured to cure a light-curable dental compound; one or more engager arms configured to engage one or more frames coupled to a dental auxiliary, where the one or more frames are part of a positioner assembly.

In any of these devices and methods, the device may engage with the frame(s) either directly or indirectly. For example, the engager arm(s) may push against the frame(s) to disconnect the frame(s) from the dental auxiliary. In some embodiments, the one or more engager arms may snap onto the frame(s) and/or may slot into the frame(s).

The dental auxiliary contact region may form a part of the tip region. The dental auxiliary contact region may have a concave surface configured to conform to the dental auxiliary. Any of these devices may include a sensor configured to sense contact and/or pressure applied by the dental auxiliary contact region against the dental auxiliary seated within the dental auxiliary contact region. Any of the devices described herein may include an output device configured to output a contact indicator and/or a pressure indicator based on the sensed contact and/or pressure applied by the dental auxiliary contact region against the dental auxiliary seated within the dental auxiliary contact region.

Also described herein are apparatuses and methods that are configured to adapt a light applicator (e.g., a “light wand”) for curing a bonding material for use with a dental auxiliary so that it may assist in separating the dental auxiliary from the frame(s) to which the dental auxiliary is attached. For example, described herein are apparatuses for adapting a light curing device for use with dental auxiliary, the apparatus including: an adapter body configured to fit over the light curing device without optically occluding a light-emitting distal tip region of the light curing device; one or more engager arms at a distal end region of the adapter body configured to engage one or more frames coupled to a dental auxiliary, where the one or more frames are part of a positioner assembly.

In some embodiments, a light curing device may be an elongate component having a distal end region and a proximal end region, and may include a light-emitting region at the distal end region and a handle at the proximal end region. An apparatus for adapting a light curing device may be configured to couple to the light curing device so that the adapter body is attached to the light curing device. In some embodiments, the apparatus for adapting a light curing device may be configured to fit over the distal end region of the light curing device by sliding over the distal end region of the light curing device. In some embodiments, the adapter body may be attached around the distal end region of the light curing device. For example, the adapter body of the apparatus for adapting a light curing device may be hinged or may be two or more pieces that engage with each other to fit over the light curing device. The apparatus may include a fitting for holding the distal end region of the light curing device. The apparatus for adapting a light curing device may be configured to interface with controls on the light curing device, such as the control to turn on/off the emission of light for curing a bonding material.

Any of the apparatuses for adapting a light curing device described herein may include a dental auxiliary contact region configured to engage with a dental auxiliary. The dental auxiliary contact region may be similar or identical to the dental auxiliary contact regions described in reference to the integrated devices for bonding a dental auxiliary to a tooth.

For example, the dental auxiliary contact region can have a concave surface. Any of these apparatuses for adapting a light curing device may include one or more sensors configured to sense contact between and/or pressure applied by the dental auxiliary contact region against the dental auxiliary seated within the dental auxiliary contact region. In some embodiments, the dental auxiliary contact region may be configured to transmit the light radiated by the light-emitting region of the light curing device. For example, the apparatuses for adapting a light curing device may include a transparent region in communication with the light-emitting distal tip region of the light curing device and/or may include a light pipe or other light transmission device to transmit light from the light-emitting distal tip region of the light curing device to a light output region of the apparatus for adapting a light curing device. The apparatus for adapting a light curing device may include one or more additional optical elements, such as filters (e.g., optical filters), lenses, mirrors, or the like. In any of these apparatuses for adapting a light curing device, the apparatus may include one or more additional light sources (e.g., LEDs).

Any of these apparatuses may include one or more actuating controls configured to extend the one or more engager arms distally to engage with the one or more frames. The actuating control(s) may be configured to actuate the engager arm(s). For example, the actuating control(s) may be configured to extend the one or more engager arms distally to engage with the one or more frames, and/or to retract the one or more engager arms proximally to disengage the one or more frame(s) from the positioner assembly. In some embodiments, the apparatus for adapting a light curing device may be configured to include one or more rotatable joints configured to enable the engager arm(s) to rotate after the one or more frames have been engaged so as to cause the one or more frames to break. The one or more actuating controls may be configured to move (e.g., translate and/or rotate the engager arm(s)). For example, any of the apparatuses for adapting a light curing device described herein may include a handle mount at a proximal end of the adapter body, and the handle mount may include an actuating control configured to actuate the one or more engager arms.

The apparatuses for bonding a dental auxiliary to a tooth described herein, including apparatuses for adapting a light curing device, may include a suction or vacuum inlet at or near a distal end of the apparatus to help capture the frame(s) and/or bonding material so that it does not end up in the subject's mouth.

Also described herein are methods of using these apparatuses for bonding a dental auxiliary to a tooth. For example, described herein are methods of using these apparatuses to bond a dental auxiliary to a tooth that may include: emitting a light from a distal tip region of a light curing device to cure a light-curable dental compound on the dental auxiliary; unlocking a release within the light curing device after the light has been emitted from the distal tip region for a cure time to permit one or more engager arms at a distal end region of the distal tip region to engage with a frame to which the dental auxiliary is attached; and actuating the one or more engager arms to engage with the frame of the dental auxiliary to uncouple the frame from the dental auxiliary after the release has been unlocked.

Any of these methods may also include sensing a pressure applied by the distal tip region of the light curing device against the dental auxiliary while emitting the light, and emitting an alert if the sensed pressure falls outside of a pressure range. The pressure range may be a predetermined range. For example, the pressure range may be 1 Pa or greater, 5 Pa or greater, 10 Pa or greater, 50 Pa or greater, 0.1 kPa or greater, 0.5 kPa or greater, 1 kPa or greater, 5 kPa or greater, 10 kPa or greater, etc. For example, unlocking the release may include unlocking the release after the light has been emitted from the distal tip region for a cure time while the sensed pressure is within the predetermined pressure range.

Alternatively or additionally, the apparatus may be configured to prevent removal of the frame from the dental auxiliary until the pressure is sufficient to hold the dental auxiliary against the tooth (e.g., to prevent disrupting the positioning and/or bonding of the dental auxiliary). For example, unlocking the release may comprise unlocking after the sensed pressure has maintained within the predetermined pressure range for the cure time.

In any of these methods and apparatuses, actuating the one or more engager arms can include actuating an actuating control. For example, actuating the one or more engager arms may include extending the one or more engager arms distally from the distal tip region to engage the frame. Actuating the one or more engager arms may include retracting the one or more engager arms proximally while the one or more engager arms are engaged with the frame. Actuating the one or more engager arms may include grabbing the frame with an engager at a distal end of each of the one or more engager arms. In any of these apparatuses, actuating the one or more engager arms may include rotating a rotatable joint to cause the one or more frames to break.

Any of these methods and apparatuses may include actuating suction from a suction port on the light curing device to remove debris from the frame.

These methods and apparatuses may sense contact and/or pressure magnitude, and in some embodiments may detect or determine the angle of contact between the distal tip region (e.g., dental auxiliary contact region) and the dental auxiliary on the tooth. For example, the method or apparatus may sense an angle between the distal tip region and the dental auxiliary and may trigger an alert if the angle is outside of a predetermined range. The predetermined range for the angle may be between 90 degrees+/−10 degrees (or +/−12 degrees, +/−15 degrees, +/−17 degrees, +/−20 degrees, +/−22 degrees, +/−25 degrees, +/−30 degrees, etc.).

In some embodiments, the methods and apparatuses described herein may monitor contact and/or pressure between the dental auxiliary and the apparatus (e.g., the distal tip region, such as the dental auxiliary contact region), and may provide an output (e.g., alert) to the user if the contact and/or pressure is not maintained within a target range. For example, these methods and apparatuses may include a visible and/or audible output if the contact and/or pressure is outside of a target range. In some embodiments, if the apparatus is not applying sufficient pressure to the dental auxiliary, the apparatus may emit a first indicator (e.g., a red light, tone), and if the apparatus is applying sufficient pressure to the dental auxiliary, the device may emit a second indicator (e.g., a green light, second tone). Sufficient pressure may be a pressure at or above a predetermined value at which the bonding material may bond the dental auxiliary to the tooth. In some embodiments, only the second indicator is provided (e.g., indicating that the pressure is within the target range; no indicator is provided if the pressure is outside of the target range). In some embodiments, a third indicator may be provided if the direction of the pressure applied is outside of a target range. Alternatively, the second indicator may indicate both that the pressure is within the target range and the direction that the pressure is applied is within the target range.

Also described herein are methods and apparatuses for guiding bonding of a dental auxiliary to a tooth that include: sensing a contact or a pressure applied by a distal tip region of a light curing device against the dental auxiliary; emitting a light from the distal tip region of the light curing device to cure a light-curable dental compound on the dental auxiliary when the sensed contact or pressure is within a predetermined pressure range; and emitting an alert if the sensed pressure falls outside of the predetermined pressure range. Sensing the contract or the pressure may include sensing contact or pressure using any appropriate sensor, such as (but not limited to): a capacitive sensor, a piezoelectric sensor, or a mechanical sensor. In some embodiments, the distal tip region may be configured to deflect when pressure is applied. For example, sensing the contact or pressure may include deflecting or deforming a spring element coupled to the distal tip region. The spring element may be part of a sensor (e.g., mechanical sensor). In any of these apparatuses, sensing the contact or pressure may include sensing contact. Sensing the contact or pressure may include sensing pressure.

The alert may be emitted from any appropriate region of the apparatus. For example, emitting the alert may comprise emitting the alert from a region of the light curing device proximal to the distal tip region, which may be easily visible to the user.

The apparatus may be configured to control the application of the light from the distal tip region of a light curing device based on the sensed contact and/or pressure. For example, emitting the light to cure the bonding material may include emitting the light while the sensed contact or pressure indicates sufficient contact between the distal tip region and the dental auxiliary.

As mentioned, the method may include unlocking a release within the light curing device after the light has been emitted from the distal tip region for a cure time, to permit one or more engager arms at a distal end region of the distal tip region to engage with a frame to which the dental auxiliary is attached. Any of these methods may include actuating the one or more engager arms to engage with the frame of the dental auxiliary to uncouple the frame from the dental auxiliary after the release has been unlocked.

Any of these methods may include engaging the dental auxiliary with a pocket (e.g., within a contact region) within the distal tip region of the light curing device. Sensing the contract or the pressure may include sensing the contact or the pressure applied by the dental auxiliary to the pocket.

As mentioned, these methods may include sensing an angle between the distal tip region and the dental auxiliary and triggering an alert if the angle is outside of a predetermined range.

For example, described herein are methods for bonding a dental auxiliary to a tooth including: sensing a contact or a pressure applied by a distal tip region of a light curing device against the dental auxiliary; emitting a light from the distal tip region of the light curing device to cure a light-curable dental compound on the dental auxiliary; emitting an alert if the sensed contact or pressure is below a threshold; unlocking a release of the light curing device after the light has been emitted from the distal tip region for a cure time to permit one or more engager arms at a distal end region of the distal tip region to engage with a frame of the dental auxiliary; and actuating the one or more engager arms to engage with the frame of the dental auxiliary to uncouple the frame from the dental auxiliary after the release has been unlocked.

Any of the apparatuses described herein may be configured to include or use software and/or firmware to assist in controlling operation of the apparatus (e.g., device) for bonding a dental auxiliary to a tooth. For example, these apparatuses may include a controller with one or more processors and circuitry for controlling or modulating operation of the apparatus, and in particular, for performing any of the methods described herein.

Also described herein are methods and apparatuses for removing dental auxiliaries. When removing dental auxiliaries from the teeth, it may be particularly challenging to prevent the dental auxiliary and/or bonding material (or fragments of the dental auxiliary and/or bonding material) from falling into the patient's mouth. Thus, any of these apparatuses for removing a dental auxiliary from a tooth may be configured to reduce or eliminate debris that may be generated when removing the attachment. For example, described herein are apparatuses for removing a dental auxiliary from a tooth while reducing the release of debris that include: a first handle pivotally connected to a second handle; a scraping jaw at a distal end region of the first handle; an occlusal gripper at a distal end region of the second handle, where the occlusal gripper includes a pocket configured to fit over the dental auxiliary; a suction lumen extending from a suction inlet at the distal end region of the first handle, at the distal end region of the second handle, or between the first and second handles, where the suction lumen is configured to aspirate debris as a dental auxiliary is being removed.

In some embodiments, an apparatus for removing a dental auxiliary may be configured as a pair of scissoring handles (e.g., scissoring members) that are pivotally connected and may include a scraper on a distal end of one of the scissoring handles and a gripper (e.g., an occlusal gripper) at the distal end of the other scissoring handle. Any of these apparatuses may include a suction connector for coupling to a source of suction on the first or second scissoring handles, e.g., at a proximal end of the first or the second scissoring handles. In some embodiments, the apparatus may include the suction source coupled to the suction lumen. The suction source may be a pump (e.g., suction pump, vacuum pump) including a suction chamber. The suction source may be a self-contained source that is integrated with the apparatus. In some embodiments, the suction source may be, for example, a battery-powered fan that pulls air to provide suction. The suction source may provide aspiration from a suction inlet that is fluidly connected to the suction lumen. The suction inlet may be positioned at a distal region of the apparatus for removing a dental auxiliary. Multiple suction inlets may be included. In some embodiments, the suction inlet may be within the pocket. For example, the suction inlet may be within the pocket and the suction lumen may extend through the second handle to the suction connector at the proximal end of the second handle. In some embodiments, the suction inlet is adjacent to the scraping jaw at the distal end region of the first handle, and the suction lumen may extend through the first handle to the suction connector at the proximal end of the first handle.

Any of these apparatuses may include one or more adhesive materials that may assist in securing and/or removing the attachment and/or bonding material from the teeth. For example, the apparatus may include a reservoir configured to hold a putty material, wherein the putty material is configured to adhere to the dental auxiliary and/or fragments of the dental auxiliary. The adhesive material (e.g., putty material) may be present within the pocket and/or a region adjacent to the pocket. In some embodiments, any of these apparatuses may include a channel coupled to the reservoir holding the putty material. The channel may be configured to convey the putty material toward the distal end region of the first or second handle.

The pocket may be configured as a dental auxiliary contact region, similar or identical to that of the apparatuses (e.g., devices) for bonding a dental auxiliary to a tooth. In some embodiments, the pocket may be oversized as compared to the dental auxiliary. In some embodiments, the pocket may include a concave surface configured to at least partially enclose the dental auxiliary.

Any of these apparatuses for removing a dental auxiliary may include one or more shields to prevent the dental auxiliary, fragments of the dental auxiliary, and/or bonding material from being ejected away from the jaws of the apparatus when removing the dental auxiliary. In some embodiments, a shield extends around the distal ends of the first handle and the second handle. In some embodiments, the shield may be a planar, curved planar, cylindrical, or tubular member that may be coupled to the apparatus. For example, the shield may be a conical barrier. The shield may be arranged around the distal end region of the apparatus for removing a dental auxiliary and/or around either a portion of the first handle and/or the second handle. For example, the apparatus for removing a dental auxiliary may include a shield, which may be referred to as a gripper shield, extending around the occlusal gripper at the distal end region of the second handle. The occlusal gripper may include a shield, which may be configured as a conical barrier.

In any of these apparatuses, the scraping jaw may include an edge configured to engage with the dental auxiliary and/or a bonding material coupling the dental auxiliary to the teeth. The scraper of the scraping jaw may be formed of a relatively hard material, e.g., having a hardness that is greater than the hardness of the bonding material. For example, the scraping jaw may include a scraper that is metallic. The scraper may be sharp.

All of the methods and apparatuses described herein, in any combination, are herein contemplated and can be used to achieve the benefits as described herein.

### A. Dental Auxiliary Placement Tools

Dental auxiliaries may be preformed features that are bonded to a patient's teeth via a bonding material (e.g., a light-curable dental compound). In some embodiments, a dental auxiliary may benefit from being bonded while applying an amount of pressure to the dental auxiliary that is sufficient to allow the bonding material to fully harden, and/or while contact and/or pressure is applied from an appropriate direction so that the bonding material bonds the dental auxiliary to the tooth in a desired orientation and/or position and/or with a desired level of integrity.

In some embodiments, the present technology provides dental auxiliary placement tools for use in bonding a dental auxiliary to a tooth. The terms “dental auxiliary placement tool,” “dental auxiliary placement device,” “dental auxiliary placement apparatus,” “dental auxiliary bonding tool,” “dental auxiliary bonding device,” “dental auxiliary bonding apparatus,” and the like may be used interchangeably herein.

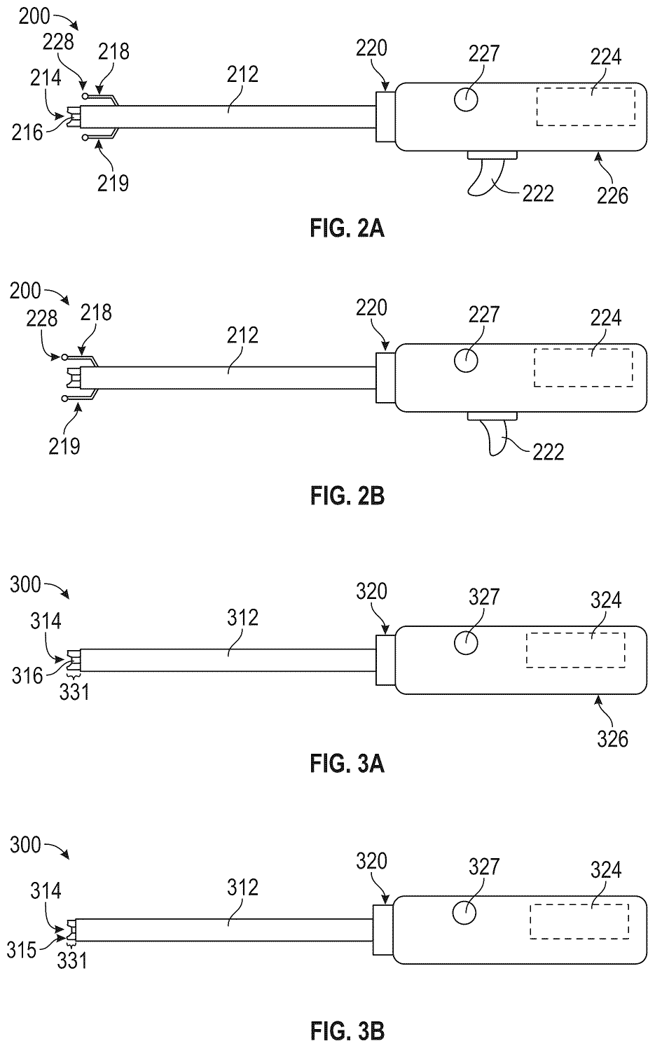

FIGS. 2A and 2B show an example of a device 200 for bonding dental auxiliaries to a tooth, in accordance with embodiments of the present technology. Dental auxiliaries may be bonded to a tooth using any suitable method, including curing of a material, activation of an adhesive, and/or any other suitable method. The device 200 includes an actuation body 212, with a handle 226 at either the proximal or distal end, and a tip region that can include an output region 216 that is configured to radiate energy having a wavelength (e.g., infrared, visible, or ultraviolet) and/or temperature that is capable of curing and/or activating a bonding material for one or more dental auxiliaries. The output region 216 may include or may be connected to an energy source that emits energy appropriate to cure the bonding material. For example, the output region 216 may include or be optically coupled to a light source (e.g., an LED) configured to emit light energy (e.g., infrared, visible, or ultraviolet light) at a wavelength appropriate to activate the bonding material. The device 200 may include one or more controls, including a control input 227 that may be on the handle 226, and may be used to turn the light source on, off, and/or to a specific duration, intensity, or wavelength.

In some embodiments, the device 200 also includes a dental auxiliary contact region 214 that is configured to interface with a dental auxiliary, which may be held in a frame, as shown in FIGS. 1C-IF. The energy output region 216 may be part of the dental auxiliary contact region 214, or it may be adjacent to the dental auxiliary contact region 214. In some embodiments, the dental auxiliary contact region 214 is configured to engage with the dental auxiliary. For example, the dental auxiliary contact region 214 may be shaped to at least partially interface with the dental auxiliary and/or with supporting components of a positioner assembly for the dental auxiliary (e.g., frame supports, frames). The dental auxiliary contact region 214 may include one or more surfaces and/or features that apply force against the dental auxiliary to hold the dental auxiliary in position against the tooth (and/or to later separate the auxiliary from the positioner assembly, e.g., after the auxiliary has been bonded to the tooth).

In some embodiments, the dental auxiliary contact region 214 also include one or more sensors (e.g., pressure sensors, contact sensors) that may detect contact and/or an applied pressure (e.g., force) between the device 200 (e.g., the dental auxiliary contact region 214) and the dental auxiliary. In some embodiments, the device 200 may include visual and/or auditory indicators that provide feedback based on data from these one or more sensors to the device user to guide the user in repositioning the device 200 until appropriate contact and/or applied pressure has been established.

Referencing FIG. 2A, the device 200 can also include one or more (e.g., a pair of) engager arms 218, 219 at the distal end region. The engager arms 218, 219 may extend distally and may each include an engager 228. The engager 228 and/or engager arms 218, 219 may generally be configured to engage with a frame and/or one or more frame supports to which the dental auxiliary is coupled. As mentioned, the frame may be part of a larger positioner assembly. In some embodiments, the engager arms 218, 219 may be actuated by an actuating control 222 on the handle 226 (e.g., shown as a trigger control) to advance the engager arms distally (e.g., as shown in FIG. 2B) so that they may engage with the frame.

In FIG. 2B the engager arms 218, 219 have been advanced distally by actuating the actuating control 222 on the handle 226. The actuating control 222 can be configured to cause the engager arms 218, 219 to engage with the frame and/or frame support coupled to the dental auxiliary while the dental auxiliary is engaged with the contact region 214.

In some embodiments, the device 200 may be configured so that the emission of energy (e.g., light energy) and/or the actuation of the engager arms 218, 219 may be controlled by a controller 224 (e.g., which may include one or more processors). In some embodiments, the operation may be controlled based on feedback from the one or more sensors, e.g., detecting contact and/or pressure. For example, the controller 224, which may be included in the handle 226 and/or the actuation body 212, may control the actuation of the energy output by preventing the transmission of the energy (e.g., by preventing activation of the energy source, by blocking emission of the energy from the output region 216) and/or by limiting the transmission of the energy (e.g., by limiting the energy source, by limiting the emission of the energy from the output region 216) until the device 200 confirms that the dental auxiliary is interfacing with the dental auxiliary contact region 2124, is being engaged against a tooth, and/or is being engaged with sufficient pressure against the tooth. Similarly, the controller 224 may be configured to prevent the engager arms 218, 219 from being actuated (e.g., to advance distally or otherwise to engage with the frame) until the controller 224 confirms one or more conditions. For example, the controller 224 may prevent actuation of the engager arms 218, 219 until the controller 224 confirms that the dental auxiliary is held within the dental auxiliary contact region 214, that the dental auxiliary has contacted a tooth, and/or that the dental auxiliary is held against the tooth within a target pressure range.

In some embodiments, the device 200 may be configured to indicate by an output device to the user that the dental auxiliary is held against the tooth within a target pressure range (e.g., a predetermined pressure range). Any of these apparatuses may include an indicator 220 that may alert the user if the dental auxiliary is held against the tooth within the predetermined pressure range. In some embodiments, the indicator 220 may be a visual indicator (e.g., an LED or other illumination source), a haptic feedback indicator (e.g., vibrating when contact is achieved), and/or an audible indicator. In some embodiments, the indicator 200 may be configured to indicate multiple states. For example, the indicator 200 may provide a first indication (e.g., a first vibration of a first frequency/duration/characteristic) when the dental auxiliary is held within the dental auxiliary contact region 214, a second indication (e.g., a second vibration at a second frequency/duration/characteristic) when the dental auxiliary first contacts a tooth, and/or a third indication (e.g., a light, a sound) when the dental auxiliary contacts the tooth with a pressure that is within a target pressure range. Any suitable combination of indication types (e.g., light, sound, haptics) may be employed for indicating different states.

FIGS. 3A and 3B illustrate an example of a device 300 that is configured to sense the force applied by the device 300 when holding a dental auxiliary against a tooth, in accordance with embodiments of the present technology. The device 300 shown in FIGS. 3A and 3B may be generally similar to the device 200 shown in FIGS. 2A and 2B. For example, the device 300 can include an actuation body 312, a proximal handle 326, a controller 327, and a dental auxiliary contact region 314 at the distal end 315 of the actuation body 312, including an output region 316. The device may also include an indicator 320, shown at the proximal end of the actuation body 312, which may be easily observed or sensed by the user.

In contrast to the example shown in FIGS. 2A and 2B, the device 300 for bonding a dental auxiliary to a tooth shown in FIGS. 3A and 3B does not include engager arms, although it may include one or more sensors sensing the force (e.g., pressure) applied by the device 300 against a dental auxiliary held within the dental auxiliary contact region 314. In the device 300, the contact region 314 may be configured to be deflected proximally based on the force applied. The applied force may deflect the dental auxiliary contact region 314 from a neutral and/or resting position 331 (FIG. 3A) to a deflected position 331′ (FIG. 3B). In some embodiments, this deflection may be applied against a biasing element (e.g., a spring) that may provide tactile feedback to the user and/or may be part of a mechanical force sensor for detecting the applied force. The sensed force may be processed by the controller 324 which may be included as part of the device 300 (e.g., within the handle 326 or actuation body 312), or may be transmitted to a remote processor. Although the disclosure focuses on particular example techniques of detecting pressure (e.g., deflection-based techniques), the disclosure contemplates any suitable technique of detecting pressure (e.g., piezoresistive and/or other pressure sensors).

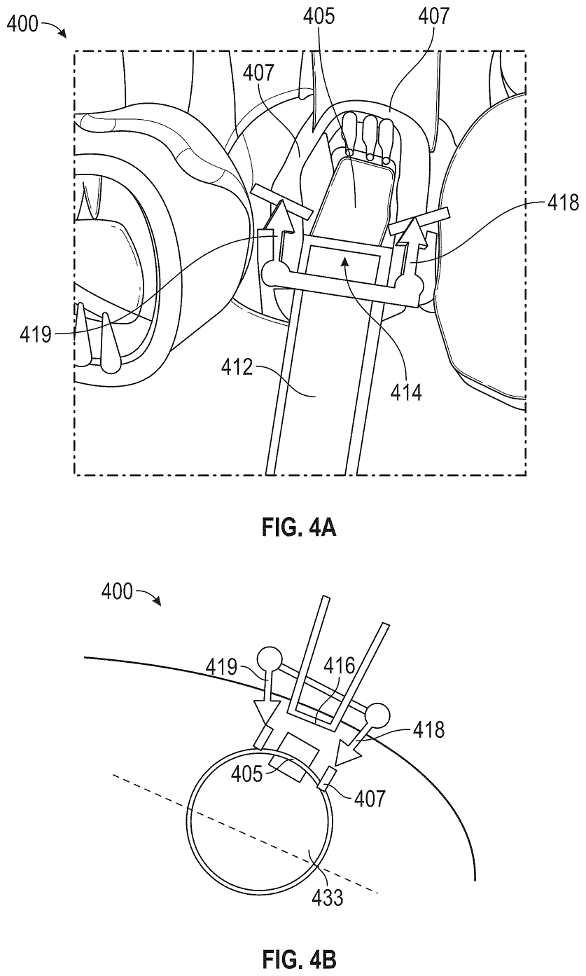

In some embodiments, an apparatus may include one or more engagers and/or engager arms, e.g., as described with respect to FIGS. 2A and 2B. The engagers and/or engager arms may engage with the frame to which the dental auxiliary is attached and may help separate the dental auxiliary from the frame. For example, FIG. 4A shows an enlarged view of a distal end portion of a device 400. The device 400 may be generally similar to the device 200 shown in FIGS. 2A and 2B. For example, the device 400 can include an actuation body 412 and a dental auxiliary contact region 414 at the distal end of the actuation body 412.

As shown in FIG. 4A, the device 400 is in contact with a dental auxiliary 405, which may be at least partially within the dental auxiliary contact region 414 of the device 400. In FIG. 4A, the distal end of the actuation body 412 includes a pair of engager arms 418, 419 that include an engager for engaging with a frame 407 to which the dental auxiliary 405 is attached. In some embodiments, the engager arms may be extended distally when the device 400 is applying force against the dental auxiliary 405 to separate the dental auxiliary 405 from the frame 407. The device 400 may be configured so that the engager arms 418, 419 are prohibited from moving to apply force to the frame 407 until the device controller confirms that the dental auxiliary 405 has been sufficiently bonded to the tooth. For example, the controller may include control logic that monitors the application of light from a light output region (or based on activation of the light control input) and prevents actuating of the engager arms 418, 419 distally (and thus preventing engagement with the frame 407) until after a predetermined curing period (e.g., of sufficient length to ensure that that the bonding material has cured sufficiently to create a suitable bond to the tooth). For example, the appropriate curing period may be 2 seconds or more, 3 seconds or more, 5 seconds or more, 10 seconds or more, 15 seconds or more, 20 seconds or more, 30 seconds or more, etc. The curing period may be set by the user or may be preset. The curing period may depend upon the wavelength used and/or the formulation of the bonding material, for example.

In some embodiments, a device may be configured so that the dental auxiliary contact region connects with the dental auxiliary in a predictable manner, which may help align the engager and/or engager arms when actuated. For example, the dental auxiliary may include a positive or subtractive feature (e.g., notch, mark, peg) that may be used to orient and/or align the device relative to the dental auxiliary. Since the configuration of the frame and dental auxiliary may be known and/or standardized, aligning the device with the dental auxiliary through the dental auxiliary contact region may align the engager arms with the frame to which the dental auxiliary is coupled, e.g., as shown in FIG. 4A.

FIG. 4B shows another example in which the device 400 includes a pair of engager arms 418, 419 that are configured to engage with the frame 407 holding the dental auxiliary 405 when the dental auxiliary 405 is held against a tooth 433 by the device 400, in accordance with embodiments of the present technology. A light output region 416 of the device 400 may apply light to the bonding material to bond the dental auxiliary 405 to the tooth 433. Once the dental auxiliary 405 is bonded to the tooth 433, the engager arms 418, 419 may be driven against the frame 407 and/or the frame supports (not shown), to separate the dental auxiliary 405 from the positioner assembly (not shown).

In some embodiments, a device may include one or more engagers in addition to (or instead of) one or more engager arms. For example, an engager may include a jaw, hook, clasp, clip, snare, hasp, etc., that contacts the frame and at least transiently attaches to it. An actuating control on the device may be used to deploy the engager and/or engager arms so that they may separate the dental auxiliary either from its own positioner assembly or from the dentition once bonded, e.g., using either mechanical, thermal, or chemical inputs.

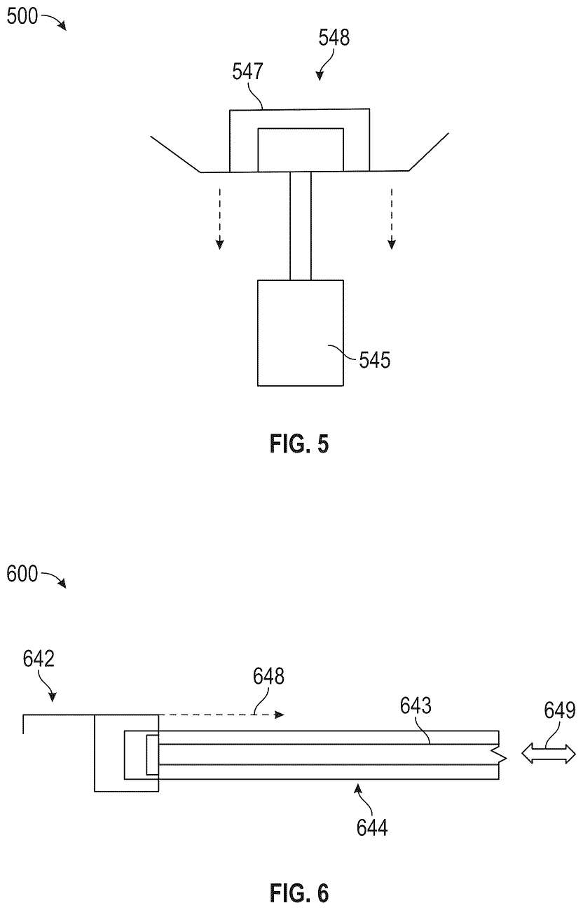

FIGS. 5-7 illustrate examples of different variations of engagers and engager arms that may be used, in accordance with embodiments of the present technology. Specifically, FIG. 5 illustrates a proximal end region of an example device 500, and FIG. 6 illustrates an engager arm 644 at a distal end region of an example device 600.

Referencing FIG. 5, the device 500 includes an actuating control 548 that is configured as a plunger-like member 547 that can be driven relative to a distal barrel portion 545 (e.g., manually by a user, robotically based on instructions from a processor or controller) to engage a distal end region, e.g., as shown in FIG. 6.

Referencing FIG. 6, the device 600 includes an engager arm 644 that may be driven proximally 648 and/or distally by actuation of a control (e.g., as shown in FIG. 5) which may pull or push on a shaft 643 (as illustrated by the double-headed arrow 649). As shown in FIG. 6, the engager arm 644 may include a hook-shaped engager 642.

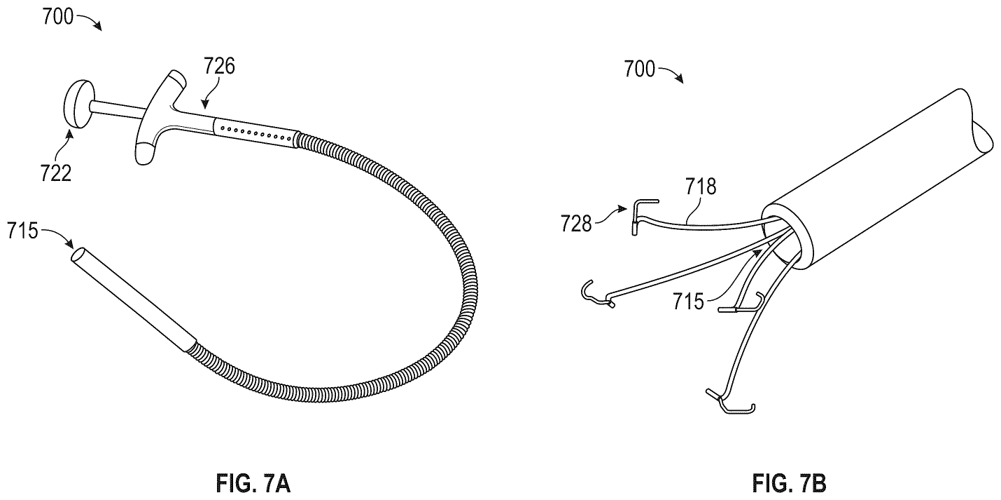

FIGS. 7A and 7B illustrate an example of a device 700 configured to engage with a frame attached to a dental auxiliary, in accordance with embodiments of the present technology. As shown in FIG. 7A, the device 700 includes an actuator having a handle 726 that includes an actuating control 722 (configured in this example as a plunger) and a distal region 715. FIG. 7B illustrates a close-up view of the distal region 715 of the device illustrated in FIG. 7A. Actuating the actuating control 722 may drive distal extension of one or more engager arms 718, each including a distal engager 728. The engagers 728 may be configured to hook onto a frame of a dental auxiliary in a positioner assembly. Some or all of the engagers 728 may have different geometries, or some or all of the engagers 728 may have the same geometry (e.g., the engagers 728 can be a set of uniform hooks).

In some embodiments, as illustrated in FIG. 7A, the device may include a flexible section in between the handle 726 and the distal region 715 to facilitate positioning of the distal region in some cases (e.g., allowing an operator to better position the distal region with one hand and independently actuate the distal engagers with the other hand). In other embodiments, the flexible section may be replaced with a rigid structure. Optionally, the distal region 715 may include a contact region (not shown), an energy output region for bonding a dental auxiliary, and/or sensors for detecting contact and/or force applied to the dental auxiliary.

In some embodiments, a method of operation of the device 700 is as follows. A user may place a positioner assembly on a patient's dentition, place the distal region 715 of the device 700 near or against a frame of a particular dental auxiliary, and then apply pressure against the dental auxiliary and the patient's tooth and further apply energy (e.g., via an output region that is integrated into the device 700 or an output region of a separate device) to bond the dental auxiliary to the patient's tooth. The user may then actuate the actuating control 722 to advance the engager arms 718 distally so as to position the engagers 728 distally beyond the frame, actuate the actuating control 722 to retract the engager arms 718 so as to cause the engagers 728 to grasp the frame, and may then pull back on the device 700 so as to sever the one or more connections between the dental auxiliary and its respective frame supports, thus disengaging the now bonded dental auxiliary from the positioner assembly.

In some embodiments, actuating of an actuating control may drive movement of one or more engager arms and/or engagers. The engager arms may be driven distally and/or retracted proximally, e.g., as described above. In some embodiments, the engager arms and/or engagers may be configured to rotate relative to a midline of the dental auxiliary, and this movement may strike and/or sever the frame supports securing the dental auxiliary to the frame.

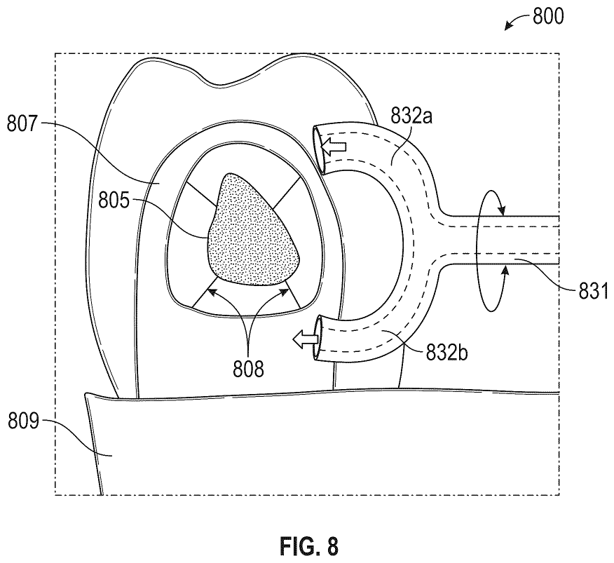

FIG. 8 illustrates an example of a distal end of a device 800 for bonding a dental auxiliary 805 to a tooth, in accordance with embodiments of the present technology. The device 800 includes a pair of engager arms 832a, 832b that may be positioned against and/or within a frame 807 for severing frame supports 808 connecting the dental auxiliary 805 to the frame 807. In FIG. 8, the engager arms 832a, 832b, which are shown as rigid projections extending distally from the device 800, may be actuated to rotate about a rotatable joint 831 to break the connection between the dental auxiliary 805 and the frame 807. In this example, the dental auxiliary 805 is attached to the frame 807 of a larger positioner assembly 809 by four frame supports 808. The engager arms 832a, 832b may be positioned such that, when the frame 807 is positioned against a tooth, the arm 832a is on a tooth-facing side of the frame 807 and the arm 832b is on an outward-facing side of the frame 807. After such positioning of the engager arms 832a, 832b, a user of the device 800 may rotate the engager arms 832a, 832b to cause the frame supports 808 to be severed and leave the dental auxiliary 805 in place on the tooth. In some embodiments, one or more of the engager arms 832a, 832b may have an engager configured to hold on to or otherwise stabilize a portion of the frame 807 prior to rotation. In such embodiments, by holding on to the frame 807, the user can maintain control over the frame 807 following the severing and can dispose of the frame 807 (e.g., reducing the likelihood of the frame 807, frame supports 808, and/or other debris from landing in undesired locations). In some embodiments, the engager arms 832a, 832b may alternatively be inserted into the open regions (e.g., apertures) between the frame supports 808, and the engager arms 832a, 832b may be rotated to cause the frame supports 808 to be severed.

In some embodiments, an apparatus may be configured to adapt or convert an existing light curing device (e.g., “light wand”) to include all or some of the features described above, including engager and/or engager arms, one or more sensors for detecting contact and/or pressure applied and/or for controlling the application of light and/or separation of the dental auxiliary from the frame and/or positioner assembly.

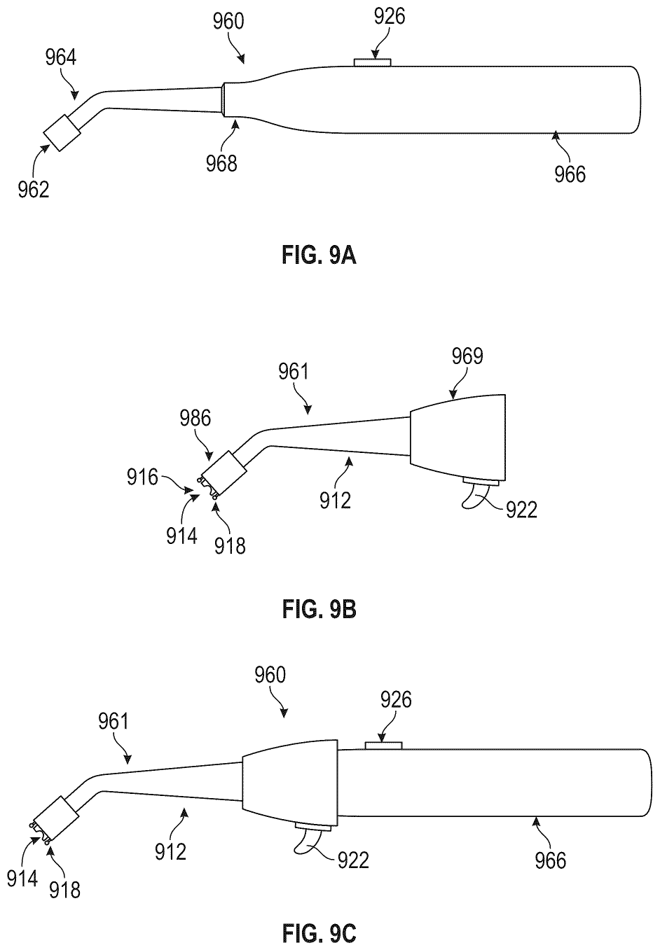

FIG. 9A illustrates an example of a light curing device 960 that may be adapted using an adapter as described herein to better control bonding of a dental auxiliary to a tooth as described herein, in accordance with embodiments of the present technology. The light curing device 960 may include a body 968 including an elongate distal end region 964 with a light emitting tip 962 and a proximate handle 966. One or more light control inputs 926 for turning on/off the light emitted from the light emitting tip 962 may typically be on the handle 966. The distal end region 964 may be curved or bent.

In some embodiments, an adapter for modifying the operation and capabilities of the light curing device 960 may include a distal end cap region that engages with the distal end region 964 of the light curing device 960. FIG. 9B schematically illustrates an example of an adapter 961 for modifying the functionality of a light curing device as described herein, in accordance with embodiments of the present technology. As shown in FIG. 9B, the adapter 961 includes a distal end cap region 986 and a light output region 916. As shown in FIG. 9C, the distal end cap region 986 is configured to fit over the distal end region 964 of the light curing device 960 so that the light emitting tip 962 of the light curing device 960 is aligned with the input to the light output region 916 of the adapter 961.

The distal end cap region 986 may include a contact region 914 configured to at least partially seat a dental auxiliary therewithin to hold the dental auxiliary against a tooth. The contact region 914 may be configured as described elsewhere herein, and may include at least one seating surface for contacting the dental auxiliary that may conform to the shape of the dental auxiliary and/or may include other features to temporarily hold the dental auxiliary (e.g., magnetic features, adhesives). For example, the contact region 914 may have a concave shape that conforms to a convex dental auxiliary. The contact region 914 may be made of a conformable material to better conform to the contours of a dental auxiliary (e.g., may be formed at least in part of a low-durometer material, such as a material having a durometer of 70 or less Shore 00, 65 or less Shore 00, 60 or less Shore 00, 55 or less Shore 00, 50 or less Shore 00, 10 or less Shore 00, 5 or less Shore 00, e.g., between 5-10 Shore 00, between 5-15 Shore 00).

The distal end cap region 986 of the adapter 961 may include one or more sensors for sensing contact and/or force (e.g., pressure) applied by the contact region 914 against the dental auxiliary. As with the other embodiments described herein, the sensors may be integrated into the contact region 914. The light output region 916 may also be integrated into the contact region 914 and/or may be adjacent to the contact region 914.

The distal end cap region 986 may also include one or more engagers and/or engager arms 918. As described herein (e.g., with respect to FIGS. 2A-8), the engager arms 918 may be configured to move upon actuation, e.g., to extend, retract, and/or rotate. In some embodiments, the engagers and/or engager arms 918 may be configured to extend out of the distal end cap region 986.

The distal end cap region 986 of the adapter 961 may be configured to secure to the distal end region 964 of the light curing device 960. In some embodiments, the distal end cap region 986 may be configured to fit onto a variety of different shapes and sizes of light curing devices. The distal end cap region 986 may include an adjustable holder, such as a clamp, brace, or press, that may secure the distal end region 964 of the light curing device 960 (or to distal end regions of other light curing devices having a variety of different sizes) to the distal end cap region 986. The outside of the distal end cap region 986 may include a release or other control to open the adjustable holder so that the distal end region 964 of the light curing device 960 can be inserted and/or coupled securely into the distal end cap region 986. The distal end cap region 986 may be slightly oversized compared to the average size of the distal end regions of different light curing devices, and/or may include an annular light collecting region, filters, and/or sensors within the distal end cap region 986 to collect light from a variety of light emitting tips of different light curing devices. Thus, the distal end cap region 986 may extend (e.g., radially) from the distal end of the light curing device when it is coupled thereto.

The adapter 916 may include a hollow adapter body 912 that connects the distal end cap region 986 to a proximal handle mount region 969. The adapter body 912 may be flexible, so as to conform to a variety of different lengths and shapes (e.g., curvatures) of light curing devices. For example, the adapter body 912 may be formed of a flexible (e.g., fabric-like) polymeric material. In some embodiments, the adapter body 912 is bendable.

The proximal handle mount region 969 may interface with the handle 966 of the light curing device 960, and may include one or more controls, such as an actuating control 922 (e.g., button, slider, switch, trigger) for activating the one or more engager arms 918 at the distal end cap region 986 (in embodiments including engager arms). For example, the actuating control 922 may include a lever or trigger that moves one or more tendons or pull wires, which may extend along the adapter body 912, causing the one or more engager arms 918 to extend and/or retract. The proximal handle mount 969 region may optionally allow engagement with the controls (e.g., light control input 926) of the light curing device 960.

Any of the adapters described herein may also include a controller for processing sensed data and/or for controlling operation of the engager arms (e.g., locking or preventing extension of the engager arms until the light control input has been activated for a sufficient amount of time to bond the light-activated bonding material and/or confirming that the contact region is in contact with the dental auxiliary within a predetermined range of pressures and/or for at least some minimum time). Any of these apparatuses may include one or more indicators to indicate contact (or lack of contact), and/or to indicate that the contact region is held against the dental auxiliary with force/pressure within a target range, as described herein.

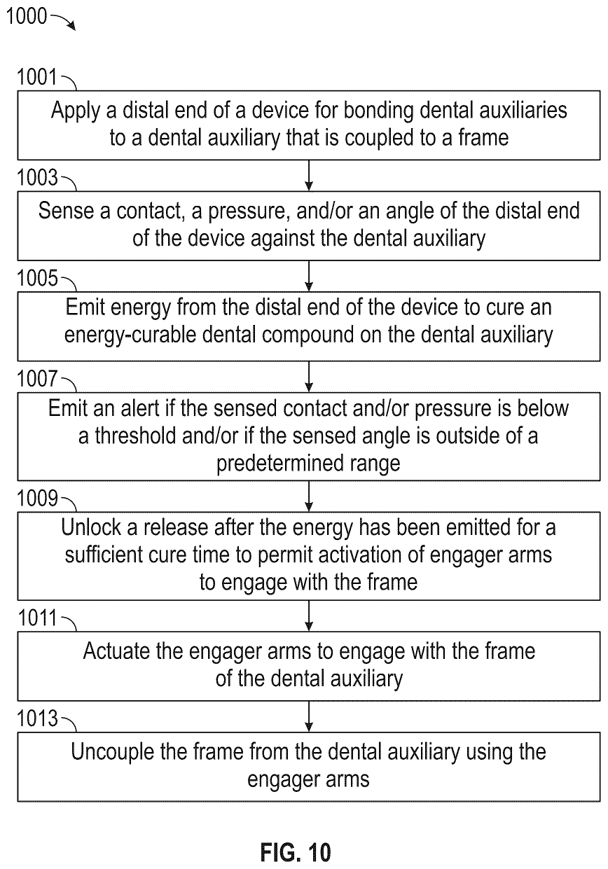

FIG. 10 schematically illustrates one example of a method 1000 for bonding a dental auxiliary to a tooth, in accordance with embodiments of the present technology. The method 1000 may be performed using an apparatus as described herein, e.g., either an integrated device for boding dental auxiliaries to a tooth or an adapter for adapting a light curing device as described herein. The method 1000 begins at block 1001 with applying a distal end of a device (e.g., a device for bonding dental auxiliaries) to a dental auxiliary that is attached to a frame and positioned on a tooth or teeth. For example, the dental auxiliary may be placed within a contact region of the device.

At block 1003, the method 1000 may optionally sense or otherwise detect a contact, a pressure, and/or an angle of the distal end of the device against the dental auxiliary. The sensed contact, pressure, and/or angle may be analyzed, e.g., by a controller (including one or more processors) and an indicator may output an indication, e.g., (light, sound, haptic vibration) to indicate an associated state (e.g., contact made, sufficient pressure applied, angle of the distal end, curing time completed). For example, the device may include an indicator on the handle that may light up to show that the correct contact, pressure, and/or angle is being applied by the device against the dental auxiliary.

At block 1005, the method 1000 can include emitting energy (e.g., a light of a wavelength and intensity configured to cause crosslinking in a bonding material) from the distal end of the device to cure an energy-curable dental compound on the dental auxiliary. The energy can be emitted when a user activates a control. In some embodiments, the control may be locked until the sensed contact and/or pressure indicates that the force being applied against the dental auxiliary by the device is greater than a minimum threshold and/or until a predetermined bonding time period has lapsed so that the attachment is adequately bonded. The light may be emitted from the distal end of the device, including from the contact region and/or a region adjacent to the contact region.

The method 1000 may optionally monitor the contact, pressure, and/or angle of the distal end of the device (e.g., as described with respect to block 1003) before and/or concurrently with the emitting of the energy in block 1005. At block 1007, an alert can optionally be triggered if the sensed contact and/or pressure is not within the target range (e.g., is below a minimum threshold) and/or the sensed angle of the device is outside of a predetermined range. The alert may be a visual alert (e.g., a flashing LED, a message on a display), an audible alert (e.g., a tone, an audible message), a haptic alert (e.g., a pulsing vibration), and/or any other suitable alert.

The method 1000 may optionally control the operation of the curing light and/or one or more engager arms of the device, e.g., by a processor. For example, the disclosed apparatuses and methods may be configured to have the engager arm functionality locked (e.g., physically, electronically, magnetically) and may be further configured to unlock such functionality at the appropriate time. In some embodiments, at block 1009, the method 1000 includes unlocking a release after the energy has been emitted for a sufficient cure time, where the unlocking of the release permits activation of the engager arms to engage with the frame.

At block 1011, the engager arms may then be actuated to engage with the frame of the dental auxiliary. For example, a control on the device may be activated manually or automatically to extend and/or rotate the engager arms to apply force against the frame.

At block 1013, the method 1000 can include uncoupling the frame from the dental auxiliary using the engager arms. For example, the forces applied to the frame by the engager arms may separate the dental auxiliary from the frame. Alternatively or additionally, the engager arms may secure the frame so that once the frame is separated from the dental auxiliary, the frame does not fall into the patient's mouth. In some embodiments, the device may then be removed from the patient's mouth, and the frame (or a portion thereof) held by the engager arms may be removed with the device and discarded.

### B. Dental Auxiliary Removal Tools

In some embodiments, the present technology provides dental auxiliary removal tools (e.g., attachment removal tools) that may be used to safely and quickly remove a dental auxiliary that is bonded to a tooth while preventing the dental auxiliary, fragments of the dental auxiliary, and/or bonding material from being released into the patient's mouth or in an otherwise uncontrolled manner. The terms “dental auxiliary removal tool,” “dental auxiliary removal apparatus,” “dental auxiliary removal device,” and the like may be used interchangeably herein. Although the dental auxiliary removal tool is described as a separate device herein for illustrative purposes, the dental auxiliary removal tool and the dental auxiliary bonding tool may be combined into a single device.

Advantages of the dental auxiliary removal tools described herein may include: reduced or no damage to the patient's tooth (e.g., no chipping, cracking, or cutting of the enamel or other portions of the tooth crown), more secure engagement with the tooth and dental auxiliary, effective removal of the entire dental auxiliary and associated bonding material (e.g., via shearing off the dental auxiliary rather than crushing the dental auxiliary, which may result in fragmentation and/or incomplete removal), effective capture of debris (e.g., the dental auxiliary and/or bonding material) to avoid loose material that may pose a choking hazard, improved patient comfort, suitability for different mouth and/or tooth sizes, easy maneuverability within the limited space of the intraoral cavity, ability to remove dental auxiliaries from hard to access teeth (e.g., posterior teeth), intuitive handling, and/or spring-loading to reduce user fatigue.

In some embodiments, the dental auxiliary removal tools herein may be configured as apparatuses for removing a dental auxiliary from a tooth while reducing the release of debris. In some embodiments, a dental auxiliary removal tool may include a first handle pivotally connected to a second handle, with a scraping jaw (also referred to herein as a “scraper”) at a distal end region of the first handle, and an occlusal gripper (also referred to herein as a “platform”) at a distal end region of the second handle. The occlusal gripper may include a pocket region that is configured to engage against (e.g., be positioned over or at least partially over) the dental auxiliary.

Any of these removal tools may include a shield or deflector that is configured to prevent or reduce the dental auxiliary and/or bonding material from being released in an uncontrolled manner, and in some embodiments, is further configured to collect such material. The shield may be a conical shield that surrounds (or at least partially surrounds) the occlusal gripper, scraper, and/or the distal end region of the tool.

In some embodiments, the removal tool may include one or more suction inlets (or ports) for applying suction through a lumen that extends from the suction inlet that may be present at the distal end region of the first handle, at the distal end region of the second handle, or between the first and second handles. The suction lumen may be configured to aspirate debris, such as the dental auxiliary and/or bonding material, as a dental auxiliary is being removed.

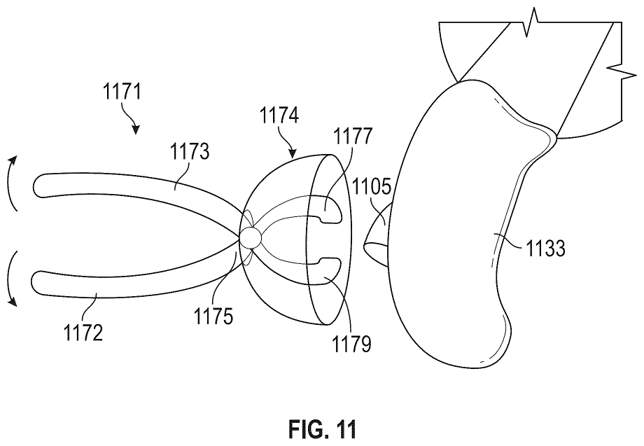

For example, FIG. 11 illustrates an example of a device 1171 configured for removing a dental auxiliary 1105 from a tooth 1133 while reducing the release of debris, in accordance with embodiments of the present technology. In FIG. 11, the device 1171 has a first handle 1172, a second handle 1173, an occlusal gripper 1179 at a distal end region of the second handle 1173, and a scraping jaw 1177 (e.g., a scraper) at a distal end region of the first handle 1172. The device 1171 also includes a shield 1174 that is coupled around the device 1171 at a shield attachment site 1175. In some embodiments, the shield attachment site 1175 may be at or proximal to a hinge pin pivotally coupling the first handle 1772 and the second handle 1173, so that the shield 1174 does not interfere with operation of the device 1171.

Debris from the dental auxiliary 1105 can be captured by the shield 1174 as the dental auxiliary 1104 is being removed. In some embodiments, the shield 1174 is configured as a clear (e.g., transparent) or translucent cup, cone, or bubble shape. The shield 1174 may be a solid structure. In other embodiments, at least a portion of the shield 1174 may be a mesh structure, with apertures of the mesh being sized to prevent passage of debris resulting from removal of the dental auxiliary 1105 but to allow the passage of, e.g., fluids. In some embodiments, the shield 1174 may be shaped like a cup that encompasses and/or encircles the distal end regions of the device 1171, and may be configured to capture debris (e.g., dental auxiliaries or portions thereof, bonding material) as a dental auxiliary 1105 is being removed.

As shown in FIG. 11, in operation, the device 1171 may be positioned adjacent to a dental auxiliary 1105 bonded on a tooth 1133. The jaws of the device 1171 may be opened so that the dental auxiliary 1105 sits within or otherwise against the occlusal gripper 1179. The occlusal gripper 1179 may be configured as a contact region that engages an occlusal side of the tooth 1133 when the device 1171 is positioned. Once positioned, the scraping jaw 1177 may then be closed by operating the first handle 1172 and/or second handle 1173, to apply force to scrape off the dental auxiliary 1105 from the tooth 1133. In some embodiments, the device 1171 may not include an occlusal gripper 1179 that grips an occlusal surface. Instead, referencing FIG. 11, both distal ends of the first handle 1172 and the second handle 1173 may be scrapers. In this case, the distal ends may be used to scrape and/or clamp the dental auxiliary 1105 to remove the dental auxiliary 1105.

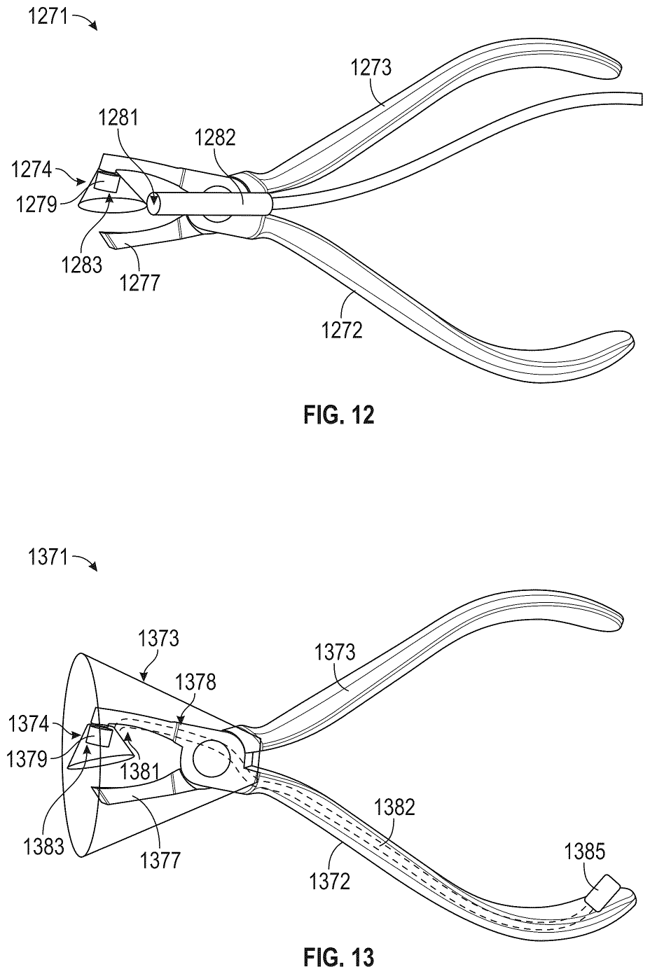

FIGS. 12-15 illustrate additional embodiments of dental auxiliary removal devices, in accordance with embodiments of the present technology. Any of the features of the embodiments of FIGS. 12-15 may be combined with each other and/or with the embodiment of FIG. 11.

FIG. 12 shows an example of a dental auxiliary removal device 1271 including a suction tip integrated onto the distal end of the device 1271 between the jaws, in accordance with embodiments of the present technology. In FIG. 12, the device 1271 is configured similar to a set of dental pliers, including a first handle 1272 and a second handle 1273 that are pivotally connected. An occlusal gripper 1279 that includes a pocket 1283 for holding a dental auxiliary and/or fragments or a dental auxiliary is at the distal end region of the first handle 1272, and a mechanical scraper 1277 is at the distal end region of the second handle 1273. The occlusal gripper 1279 is surrounded by a shield 1274 (e.g., a cup-shaped element). In some embodiments, the geometry of the occlusal gripper 1279 can be configured to engage the occlusal tooth surface. In some embodiments, the occlusal gripper 1279 can include an elastic and resilient material that conforms to at least a portion of the geometry of the occlusal tooth surface when it is pressed against the occlusal tooth surface so as to increase engagement.

The device 1271 includes a suction inlet 1281 that is connected to a suction lumen 1282. In the illustrated embodiment, the suction inlet 1281 is positioned between the first handle 1272 and the second handle 1723. The suction lumen 1282 may be connected to a source of negative pressure (e.g., a vacuum source), which may be a free-standing aspirator (e.g., a fan with a filter), a vacuum pump, etc. Suction may be provided via the suction lumen 1282 prior to, during, and/or after removal of a dental auxiliary such that debris (including the dental auxiliary or portions thereof) are aspirated away through the suction lumen 1282.

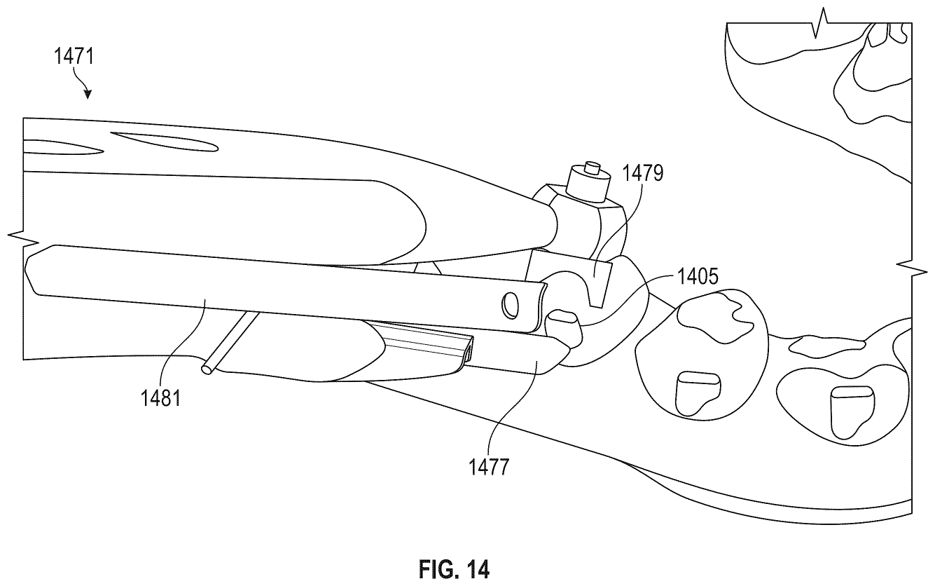

FIG. 13 illustrates a dental auxiliary removal device 1371 having a suction inlet integrated directly into one or both jaws (e.g., the jaw 1378), in accordance with embodiments of the present technology. A shield 1373 extends around both jaws of the device 1371 (e.g., similar to the example shown in FIG. 11). The device 1371 can include a first handle 1372 pivotally coupled to a second handle 1373, with an occlusal gripper 1379 (e.g., that may include a pocket 1383) at the distal end region of the first handle 1372, and a scraping jaw 1377 at the distal end region of the second handle 1373. The occlusal gripper 1379 may optionally include a separate, smaller cup or shield 1374.

In FIG. 13, a suction inlet 1381 is located in the distal portion of the first handle 1372, e.g., proximate to the occlusal gripper 1379 and pocket 1381. The suction lumen 1382 can be connected to a suction lumen 1382 that extends through the first handle 1372. In some embodiments, the device 1371 may also include a suction connector 1385 at the proximal end of the first handle 1372 for connecting the suction lumen 1382 to a source of negative pressure.

Any of the dental auxiliary removal tools described herein may or may not include suction elements. The dental auxiliary removal tools that do include suction may also include a control for turning suction on/off and/or increasing/decreasing the suction. In some embodiments, the suction control may be provided on a handle of the tool.

FIG. 14 shows an enlarged view of a distal end region of a dental auxiliary removal device 1471, in accordance with embodiments of the present technology. The device 1471 may be generally similar to the embodiment shown in FIG. 12. For example, the device 1471 can include a first jaw having an occlusal gripper 1479 that has been hollowed out to form a pocket, a scraper 1477, and a suction inlet opening into a suction lumen 1481. The device 1471 may be used to remove one or more dental auxiliaries 1405.

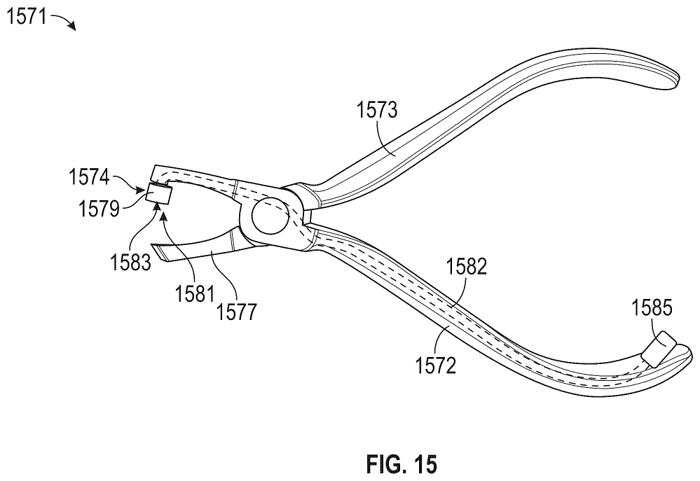

FIG. 15 illustrates a dental auxiliary removal device 1571, in accordance with embodiments of the present technology. The device 1571 includes a first handle 1572 pivotally coupled to a second handle 1572, an occlusal gripper 1579 at a distal end region of the first handle 1572, a scraper 1577 at a distal end region of the second handle 1573, and a suction inlet 1581 positioned within a pocket 1583 of the occlusal gripper 1579. The occlusal gripper 1579 can optionally include a shield 1574 surrounding the pocket 1583, opposite from the scraper 1577. The suction inlet 1581 may be connected to a suction lumen 1582 running through the first handle 1572 and may include a suction connector 1585.

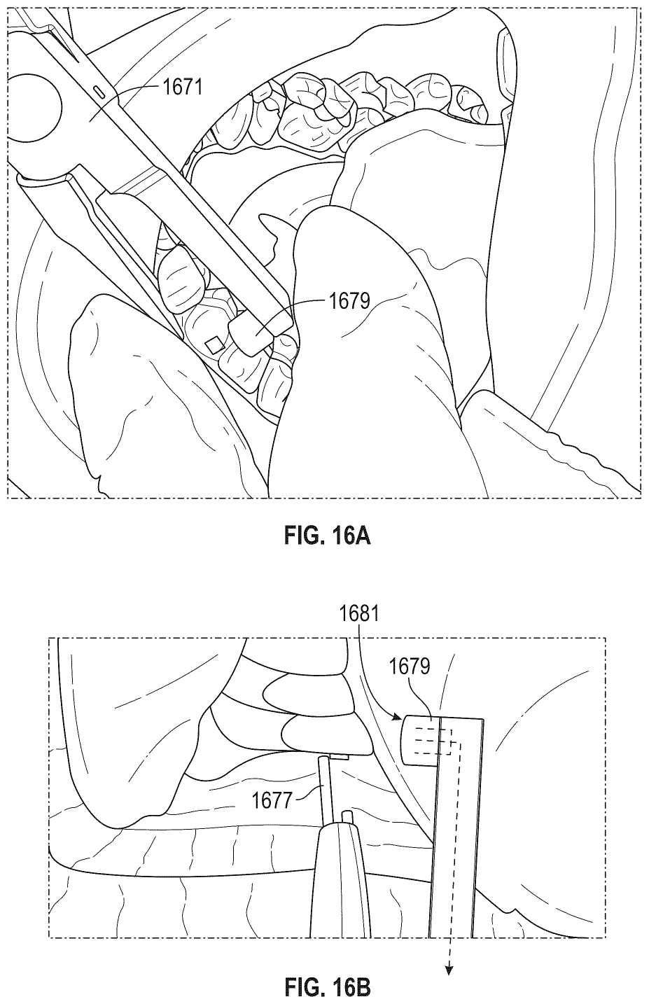

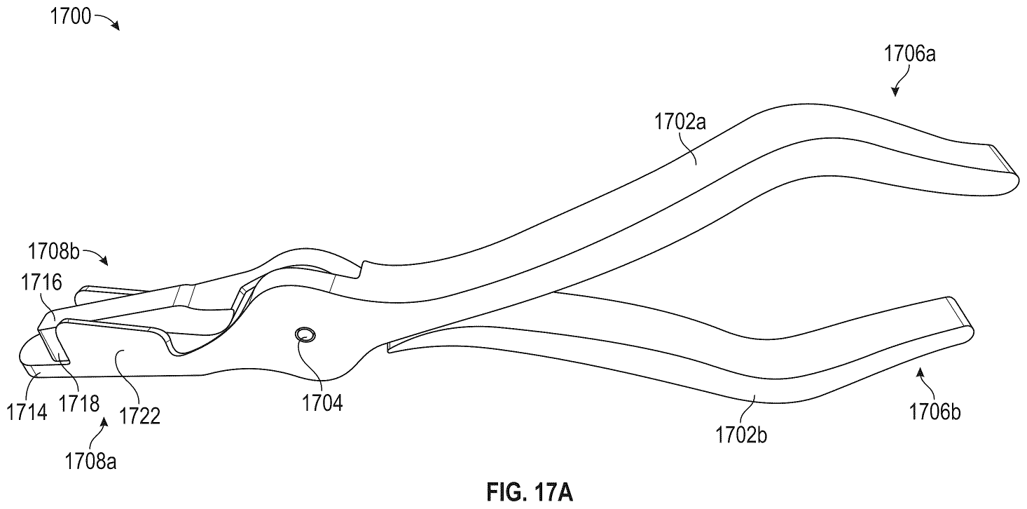

FIGS. 16A and 16B illustrate an example of a dental auxiliary removal device 1671 in use, in accordance with embodiments of the present technology. In this example, an occlusal gripper 1679 of the device 1671 is positioned against an occlusal surface of a tooth with dental auxiliary that is to be removed, and a scraping jaw 1677 is driven against the dental auxiliary such that when the jaws of the device 1671 are brought together, the dental auxiliary is removed. Suction can be applied through a suction inlet 1681 that may be present on the occlusal gripper 1679 (e.g., via a hollowed pocket or cavity formed in the occlusal gripper 1679) to secure the dental auxiliary, fragments of the dental auxiliary, and/or bonding material as it is removed by the scraping jaw 1677. A shield may also or alternatively be used to capture material removed.

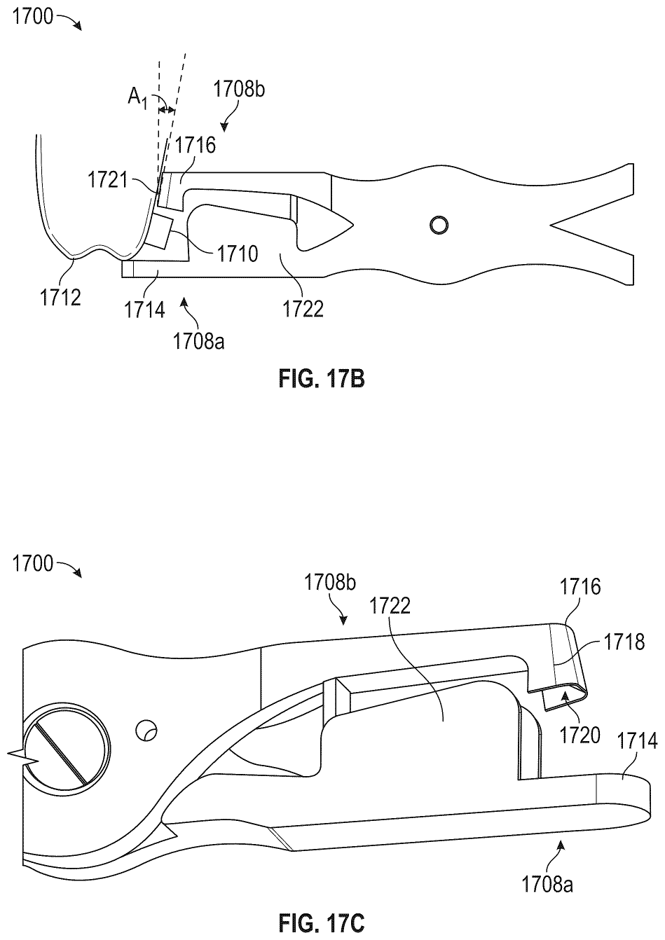

FIGS. 17A-20D illustrate additional examples of dental auxiliary removal devices. Any of the features of the embodiments of FIGS. 17A-20D may be combined with each other and/or with any of the other embodiments described herein, such as the embodiments of FIGS. 11-16B.

FIGS. 17A-17C illustrate a dental auxiliary removal device 1700, in accordance with embodiments of the present technology. Specifically, FIG. 17A is a perspective view of the device 1700, FIG. 17B is a side view of a distal portion of the device 1700, and FIG. 17C is a perspective view of the distal portion of the device 1700.

Referring first to FIG. 17A, the device 1700 includes a first elongate member 1702a and a second elongate member 1702b that are pivotally coupled to each other at a hinge 1704. The first elongate member 1702a includes a proximal end 1706a and a distal end 1708a, and the second elongate member 1702b includes a proximal end 1706b and a distal end 1708b. The proximal ends 1706a, 1706b can be configured as handles for an operator to manipulate the device 1700. The distal ends 1708a, 1708b can be configured as opposing jaws for engaging and removing a dental auxiliary 1710 from a patient's tooth 1712 (FIG. 17B). For example, the operator can move the proximal ends 1706a, 1706b apart from each other to separate the distal ends 1708a, 1708b, e.g., to open the distal ends 1708a, 1708b around the dental auxiliary 1710. The operator can then move the proximal ends 1706a, 1706b toward each other, e.g., to close the distal ends 1708a, 1708b around the dental auxiliary 1710 to apply force to remove the dental auxiliary 1710 from the tooth 1712. Optionally, the device 1700 can include a spring element (not shown) that biases the first and second elongate members 1702a, 1702b in either an open configuration or a closed configuration, if desired (FIG. 17A illustrates the closed configuration and FIGS. 17B and 17C illustrate the open configuration).

As best seen in FIGS. 17B and 17C, the distal end 1708a of the first elongate member 1702a can include a platform 1714 that is configured to support the tooth 1712. The platform 1714 can have a generally flattened upper surface that contacts the occlusal surface of the tooth 1712. The distal end 1708b of the second elongate member 1702b can include a scraper 1716 that is configured to apply force to the dental auxiliary 1710 to shear, scrape, cut, or otherwise separate the dental auxiliary 1710 from the tooth 1712 while the tooth 1712 is resting on the platform 1714. The scraper 1716 can be integrally formed with the second elongate member 1702b or can be a discrete component that is coupled to the second elongate member 1702b (e.g., via fasteners, adhesives, welding). As shown in FIG. 17C, the scraper 1716 can be a hollow or recessed structure including sidewalls 1718 defining an interior cavity 1720 configured to receive at least a portion of the dental auxiliary 1710 therein. Accordingly, the scraper 1716 may apply forces to the dental auxiliary 1710 primarily or entirely via the lower edges of the sidewalls 1718. The sidewalls 1718 may be arranged in a U-shaped configuration to apply forces to the front and sides of the dental auxiliary 1710. Optionally, the lower edges of the sidewalls 1718 may be sharpened, e.g., to facilitate cutting into the dental auxiliary 1710 and/or bonding material during the removal process.

In some embodiments, as shown in FIG. 17B, the distal surface 1721 of the scraper 1716 (FIG. 17B) can be angled in a proximal direction. Stated differently, an angle A₁ of the distal surface 1721 relative to vertical (e.g., the direction perpendicular to the upper surface of the platform 1714) can be greater than 0°, such as greater than or equal to 5°, 10°, 15°, 20°, 30°, 40°, or 45°. This configuration may be beneficial, for example, to reduce the likelihood of the scraper 1716 scratching the surface of the tooth 1716 when the scraper 1716 is brought down against the dental auxiliary 1710, thereby avoiding damage to the enamel of the tooth. In other embodiments, however, the distal surface 1721 can be vertical (e.g., the angle A₁ is 0°) or angled in a distal direction, if appropriate. In some embodiments, a distal surface of the scraper 1716 may be covered with a softer, less rigid material (e.g., an elastomer, a plastic piece) or a coating, for example to reduce the possibility of enamel damage.

In some embodiments, the device 1700 includes features configured to capture debris generated during removal of the dental auxiliary 1710 from the tooth 1712. Such debris may include the dental auxiliary 1710 (or portions thereof) and/or the bonding material coupling the dental auxiliary 1710 to the tooth 1712. As noted elsewhere herein, it may be desirable to prevent the debris from landing in the patient's mouth (e.g., which may present a choking hazard) or otherwise flying uncontrollably away from the tooth 1712 (e.g., which may cause injury to the patient or the clinician). For example, in the illustrated embodiment, the distal end 1708a of the first elongate member 1702a includes a pair of sidewalls 1722. The sidewalls 1722 can be located on opposite sides of the first elongate member 1702a, and may extend vertically or substantially vertically upward toward the distal end 1708b of the second elongate member 1702b. The sidewalls 1722 may be located proximal to the platform 1714 and may terminate before the distalmost tip of the first elongate member 1702a, e.g., to avoid interfering with placement of the tooth 1712 on the platform 1714. The sidewalls 1722 may be integrally formed with the first elongate member 1702a or may be discrete components that are coupled to the first elongate member 1702a (e.g., via fasteners, adhesives, welding).

The geometry and location of the sidewalls 1722 of the first elongate member 1702a and the sidewalls 1718 of the scraper 1716 can be configured such that when the scraper 1716 is brought into contact with the dental auxiliary 1710, the dental auxiliary 1710 and/or associated bonding material is directed into the space between the sidewalls 1722 of the first elongate member 1702a, thereby capturing the dental auxiliary 1710 and/or bonding material within the device 1700. As can be seen in FIG. 17A, when the device 1700 is closed, the sidewalls 1722 of the first elongate member 1708a and the sidewalls 1718 of the scraper 1716 can collectively form a chamber that partially or fully encloses the captured dental auxiliary 1710 and/or bonding material. The device 1700 can then be removed from the patient's mouth together with the captured dental auxiliary 1710 and/or bonding material.

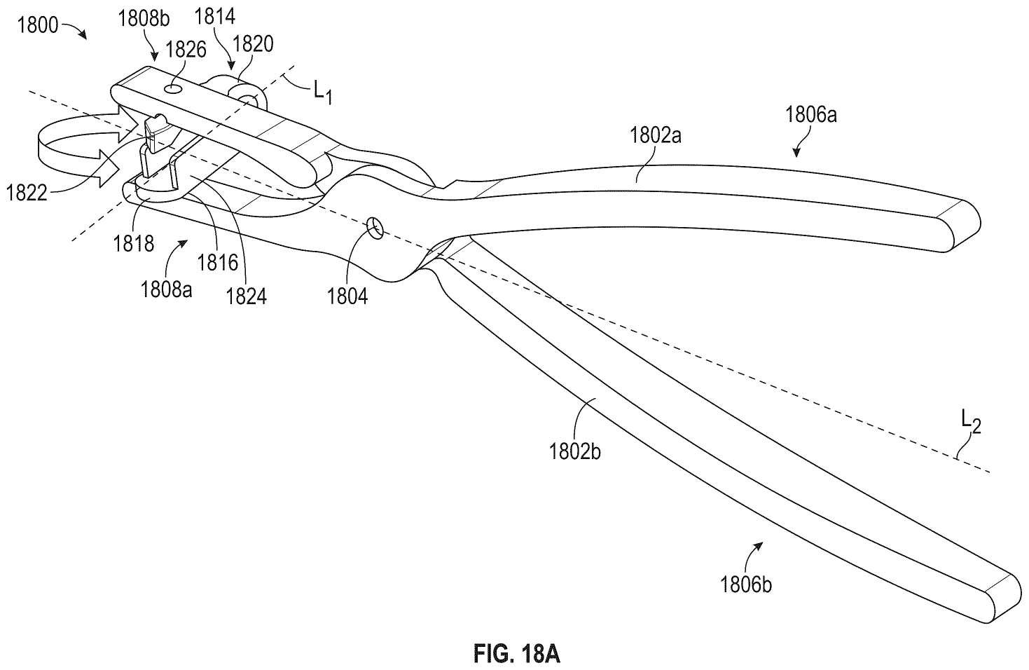

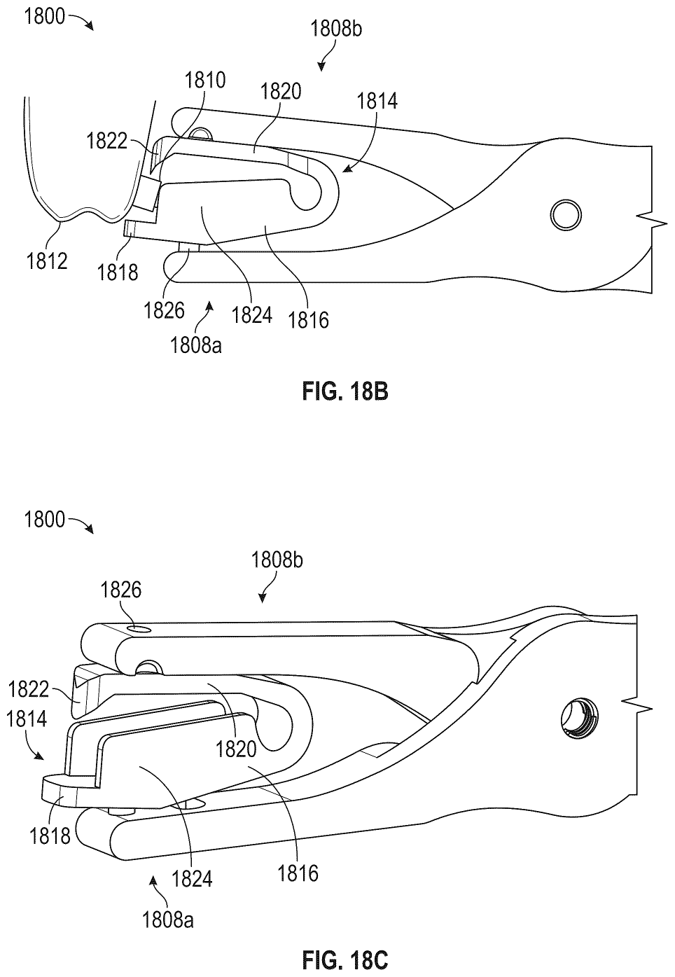

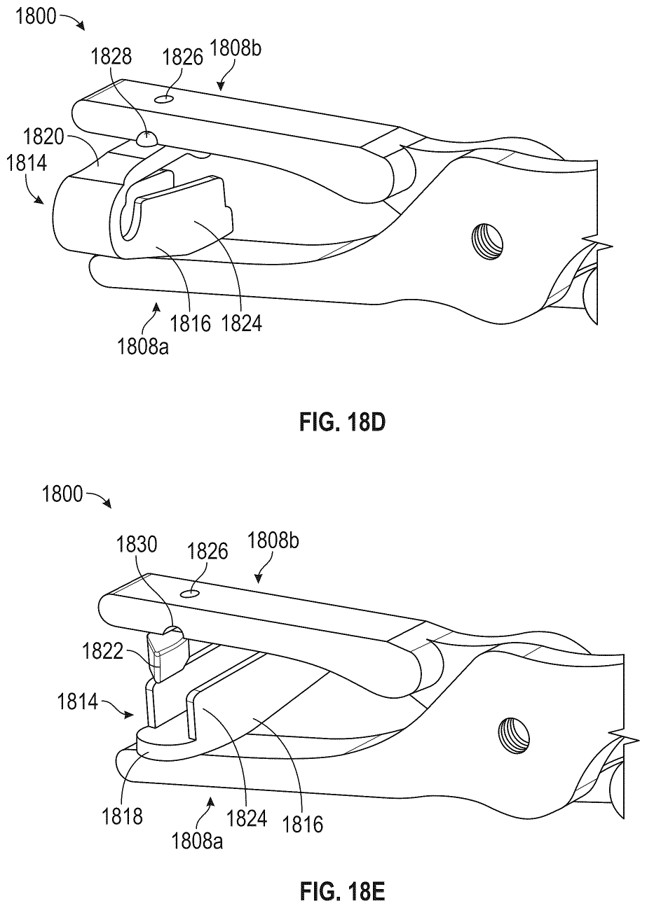

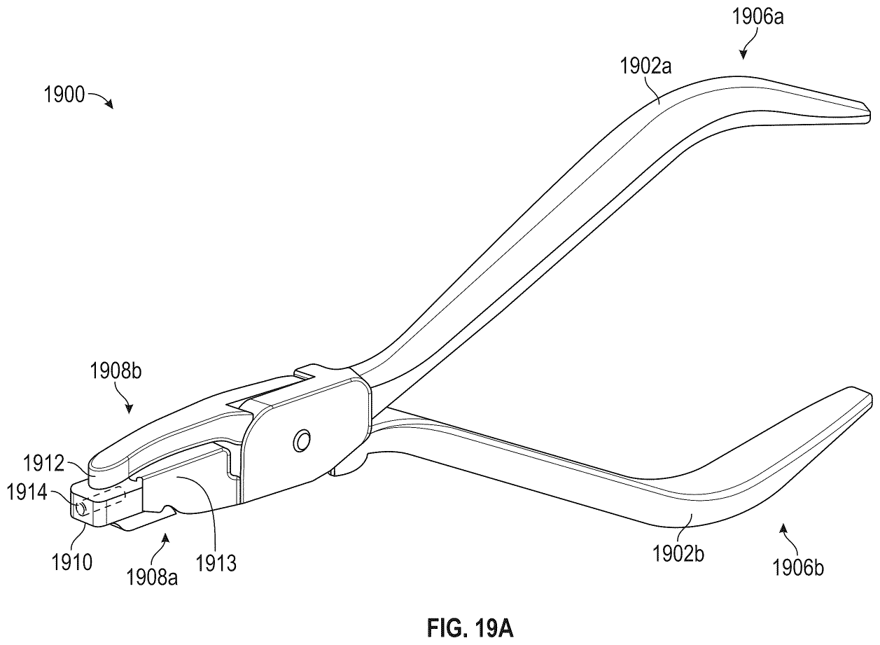

FIGS. 18A-18E illustrate a dental auxiliary removal device 1800, in accordance with embodiments of the present technology. Specifically, FIG. 18A is a perspective view of the device 1800, FIG. 18B is a side view of a distal portion of the device 1800, and FIGS. 18C-18E are perspective views of the distal portion of the device 1800.

Referring first to FIG. 18A, the device 1800 includes a first elongate member 1802a and a second elongate member 1802b that are pivotally coupled to each other at a hinge 1804. The first elongate member 1802a includes a proximal end 1806a and a distal end 1808a, and the second elongate member 1802b includes a proximal end 1806b and a distal end 1808b. The first and second elongate members 1802a, 1802b may be operated similarly to the corresponding components of FIGS. 17A-17C, e.g., the proximal ends 1806a, 1806b may serve as handles for opening and closing the distal ends 1808a, 1808b around a dental auxiliary 1810 on a tooth 1812 (FIG. 18B). Optionally, the device 1800 can include a spring element (not shown) that biases the first and second elongate members 1802a, 1802b in either an open configuration or a closed configuration, if desired (FIGS. 18A-18E illustrate the open configuration).

In the illustrated embodiment, the device 1800 includes a rotatable assembly 1814 coupled to the distal ends 1808a, 1808b of the first and second elongate members 1802a, 1802b. As shown in FIG. 18B, the rotatable assembly 1814 can include a lower portion 1816 including a platform 1818 configured to support the tooth 1812, and an upper portion 1820 including a scraper 1822 configured to apply force to the dental auxiliary 1810 on the tooth 1812. The lower portion 1816 and the upper portion 1820 can be connected to each other to form a set of opposing jaws for receiving the dental auxiliary. The platform 1818 and the scraper 1822 can be identical or generally similar to the platform 1714 and scraper 1716 of the device 1700 of FIGS. 17A-17C. For instance, when the distal ends 1808a, 1808b of the device 1800 are closed, the upper portion 1820 and lower portion 1816 can move toward each other, thus causing the scraper 1822 to apply force to the dental auxiliary to shear, scrape, cut, or otherwise separate the dental auxiliary from the tooth while the tooth is resting on the platform 1818. Similar to the device 1700 of FIGS. 17A-17C, in some embodiments, the surface of the scraper configured to be directed toward the auxiliary may be angled by an angle (e.g., similar to A₁ in FIG. 17B) relative to vertical (e.g., the direction perpendicular to the upper surface of the platform), and this angle can be greater than 0°, such as greater than or equal to 5°, 10°, 15°, 20°, 30°, 40°, or 45°. In some embodiments, the platform 1818 and the scraper 1822 are integrally formed with the lower portion 1816 and upper portion 1820, respectively. In other embodiments, one or both of the platform 1818 or scraper 1822 may be discrete components that are coupled to the lower portion 1816 and upper portion 1820, respectively (e.g., via fasteners, adhesives, welding).

Optionally, the lower portion 1816 of the rotatable assembly 1814 can include a pair of sidewalls 1824 that are identical or generally similar to the sidewalls 1722 of the device 1700 of FIGS. 17A-17C. For instance, the sidewalls 1824 may extend vertically or substantially upward toward the upper portion 1820 to capture debris generated during removal of the dental auxiliary. When the scraper 1822 is brought into contact with the dental auxiliary, the dental auxiliary and/or associated bonding material can be directed into the space between the sidewalls 1824, thereby capturing the dental auxiliary and/or bonding material within the device 1800.

Referring to FIGS. 18A-18E together, the rotatable assembly 1814 can be pivotally coupled to the distal ends 1808a, 1808b via a hinge 1826 to allow the rotatable assembly 1814 (e.g., the scraper 1822 and the platform 1818) to rotate to a plurality of different orientations relative to the rest of the device 1800 (e.g., as indicated by the arrows in FIG. 18A). As shown in FIG. 18A, the rotatable assembly 1814 may be rotated such that the longitudinal axis L₁ of the rotatable assembly 1814 is at any desired angle relative to the longitudinal axis L₂ of the rest of the device 1800. For example, the rotatable assembly 1814 can be rotated such that the longitudinal axis L₁ of the rotatable assembly 1814 is parallel or substantially parallel to the longitudinal axis L₂ of the rest of the device 1800, e.g., the scraper 1822 and platform 1818 are located at the distalmost end of the device 1800 as shown in FIGS. 18B and 18C. This configuration may be used, e.g., for removing dental auxiliaries from anterior teeth and/or other easily accessible teeth. As another example, the rotatable assembly 1814 can be rotated such that the longitudinal axis L₁ of the rotatable assembly 1814 is perpendicular to or substantially perpendicular to the longitudinal axis L₂ of the rest of the device 1800, e.g., the scraper 1822 and platform 1818 are proximal to the distalmost end of the device 1800 as shown in FIGS. 18A, 18D, and 18E. This configuration may be used, e.g., for removing dental auxiliaries located on posterior teeth and/or teeth that are obstructed or otherwise difficult to access. Other angles are also possible, e.g., the rotatable assembly 1814 may be rotated so that the longitudinal axis L₁ of the rotatable assembly 1814 is at an angle of 5°, 10°, 15°, 20°, 30°, 40°, 45°, 50°, 60°, 70°, or 80° relative to the longitudinal axis L₂ of the rest of the device 1800. This configuration allows the operator to adjust the orientation of the rotatable assembly 1814 based on the location of the dental auxiliary, location of the tooth, ergonomic considerations, patient comfort, etc.

Optionally, the device 1800 can include locking features for fixing the rotatable assembly 1814 at one or more desired orientations relative to the rest of the device 1800. In the illustrated embodiment, for instance, the rotatable assembly 1814 includes a protrusion 1828 (e.g., a pin, post, button—see FIG. 18D) that mates with a corresponding recess 1830 in the distal end 1808b (e.g., a hole, groove, channel—see FIG. 18E) to lock the rotatable assembly 1814 at a 90° angle relative to the rest of the device 1800. This may be advantageous, for example, to stabilize the rotatable assembly 1814 during removal of the dental auxiliary. The locking feature may be configured in many different ways, e.g., the recess 1830 may be located on the rotatable assembly 1814 and the protrusion 1828 may be located on the distal end 1808b, other types of locking features may be used, the rotatable assembly 1814 may alternatively or additionally be locked at other angles (e.g., a 0° angle relative to the rest of the device 1800), etc.

In some embodiments, the device 1800 includes controls for controlling the rotation of the rotatable assembly 1814. Such controls may include, for example, one or more dials, knobs, levers, buttons, pull wires, etc. The controls may be located on one or both of the proximal ends 1806a, 1806b. Optionally, the controls may also control locking and unlocking of the rotatable assembly 1814 (in embodiments where such locking functionality is present). In some embodiments, the rotation may simply be adjusted by directly rotating the rotatable assembly 1814, in which case a separate control mechanism may not be required.

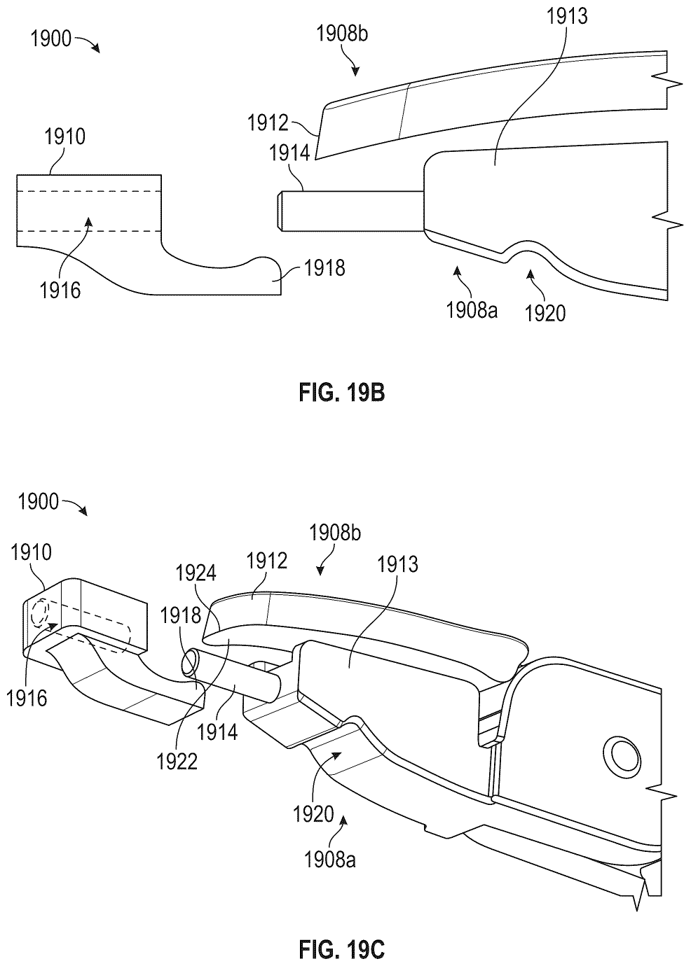

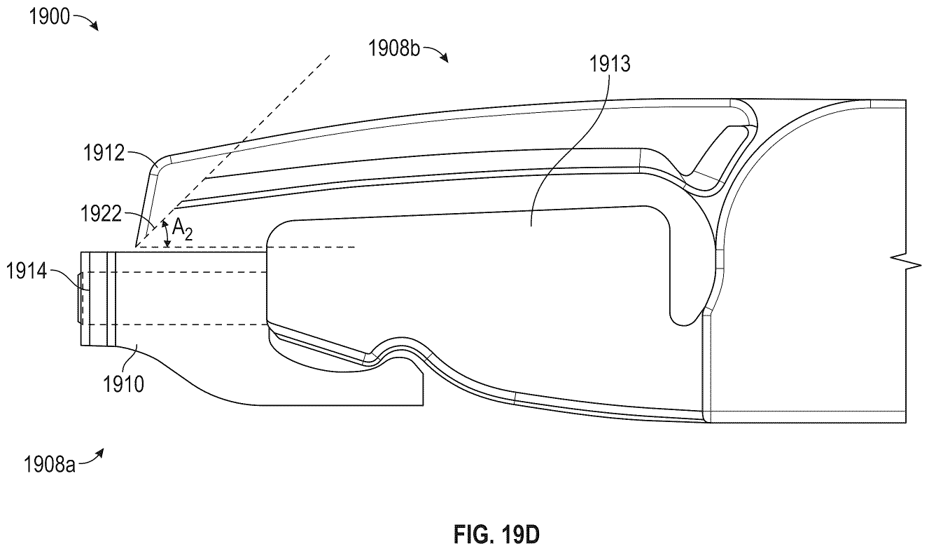

FIGS. 19A-19D illustrate a dental auxiliary removal device 1900, in accordance with embodiments of the present technology. Specifically, FIG. 19A is a perspective view of the device 1900, FIGS. 19B and 19C are exploded views of a distal portion of the device 1900, and FIG. 19D is a side view of the distal portion of the device 1900. The device 1900 may be generally similar to the device 1700 of FIGS. 17A-17C and the device 1800 of FIGS. 18A-18E. For example, as shown in FIG. 19A, the device 1900 includes a first elongate member 1902a and a second elongate member 1902b that are pivotally coupled to each other at a hinge 1904, with respective proximal ends 1906a, 1906b that serve as handles for opening and closing respective distal ends 1908a, 1908b around a dental auxiliary on a tooth.

The distal end 1908a of the first elongate member 1902a can include a platform 1910 that is configured to support the tooth. The distal end 1908b of the second elongate member 1902b can include a scraper 1912 that is configured to apply force to the dental auxiliary to shear, scrape, cut, or otherwise separate the dental auxiliary from the tooth while the tooth is resting on the platform 1910. The platform 1910 and scraper 1912 may be generally similar to the corresponding components described with respect to FIGS. 17A-18E. For instance, when the distal ends 1908a, 1908b of the device 1900 are closed, the scraper 1912 can apply force to the dental auxiliary to shear, scrape, cut, or otherwise separate the dental auxiliary from the tooth while the tooth is resting on the platform 1910. Optionally, the distal end 1908a can include a pair of sidewalls 1913 that are identical or generally similar to the sidewalls 1722 of the device 1700 of FIGS. 17A-17C. For instance, the sidewalls 1913 may extend vertically or substantially upward to capture debris generated during removal of the dental auxiliary. When the scraper 1912 is brought into contact with the dental auxiliary, the dental auxiliary and/or associated bonding material can be directed into the space between the sidewalls 1913, thereby capturing the dental auxiliary and/or bonding material within the device 1900.

In some embodiments, the platform 1910 is a discrete component that is coupled to the distal end 1908a, e.g., via mechanical fit (e.g., snap fit, interference fit, interlocking features), fasteners, adhesives, magnets, etc. For instance, as best seen in FIGS. 19B and 19C, the distal end 1908a of the first elongate member 1902a can include a post 1914 that fits within a corresponding hole 1916 in the platform 1910, thereby coupling the platform 1910 to the distal end 1908a in a set position and orientation. The platform 1910 can also include a set of proximal protrusions 1918 that mate with corresponding recesses 1920 in the distal end 1908a via snap fit to secure the platform 1910 to the distal end 1908a. Prior to removing a dental auxiliary using the device 1900, the platform 1910 can be coupled to the distal end 1908a of the first elongate member 1902a. The platform 1910 may be decoupled from the distal end 1908a after the dental auxiliary removal procedure is complete, e.g., for cleaning or for disposal.

The platform 1910 can be made of a different material than the rest of the device 1900. In some embodiments, for example, the platform 1910 is made out of a polymeric material (e.g., polycarbonate or other suitable plastic), while the rest of the device 1900 is made out of a metallic material (e.g., steel). In this example, the two materials have different rigidities (e.g., the platform 1910 being less rigid than the rest of the device 1900). This can be advantageous for providing a softer tooth-contacting surface, e.g., to improve patient comfort during the removal procedure and/or avoid damage to the tooth. Optionally, the platform 1910 can be made out of a transparent or translucent material to facilitate visualization of the dental auxiliary and tooth, while the rest of the device 1900 may be made out of an opaque material. In some embodiments, the platform 1910 is made out of a sterilizable (e.g., autoclavable) material, e.g., if the platform 1910 is intended to be reusable. In other embodiments, however, the platform 1910 may be a single use component that is discarded rather than being sterilized for reuse.

Referring to FIG. 19C, in some embodiments, the scraper 1912 is a solid structure including a flattened lower surface 1922 that contacts the dental auxiliary to apply force thereto. Optionally, the edges 1924 of the scraper 1912 may be sharpened to cut into the dental auxiliary and/or bonding material during the removal process. In some embodiments, the use of a solid scraper 1912 having a flattened lower surface 1922 instead of a hollow scraper (e.g., as illustrated in FIGS. 17A-17C) may reduce the likelihood of the edges 1924 catching onto and damaging the surface of the tooth when the scraper 1912 is brought down against the dental auxiliary.

FIG. 19D illustrates another configuration for the scraper 1912. As shown in FIG. 19D, the distal region of the lower surface 1922 of the scraper 1912 may be angled or curved (e.g., concave). This configuration may be advantageous, for example, to improve deflection of debris generated by removal of the dental auxiliary between the sidewalls 1913. The angle A₂ of the lower surface 1922 of the scraper 1922 (e.g., relative to horizontal) can be selected to achieve the desired balance between capturing debris and avoiding damaging the tooth, e.g., a larger angle A₂ may provide better capture of debris while a smaller angle A₂ may reduce the likelihood of the scraper 1922 catching on the tooth surface. In some embodiments, the angle A₂ is greater than or equal to 0°, 5°, 10°, 15°, 20°, 30°, 40°, 45°, 50°, 60°, 70°, or 80°; and/or is less than or equal to 90°, 80°, 70°, 60°, 50°, 45°, 40°, 30°, 20°, 15°, 10°, or 5°.

Although FIGS. 17A-19D illustrate dental auxiliary removal devices with integrally formed sidewalls for capturing debris (e.g., sidewalls 1722, 1824, 1913), in other embodiments, the sidewalls may be omitted. In such embodiments, a removable sleeve may be temporarily coupled to the device, where the sleeve includes sidewalls to capture debris. The removable sleeve may be a reusable (e.g., sterilizable) component or may be a single use disposable component. The removable sleeve can be made out of any suitable material, such as a polymeric material.

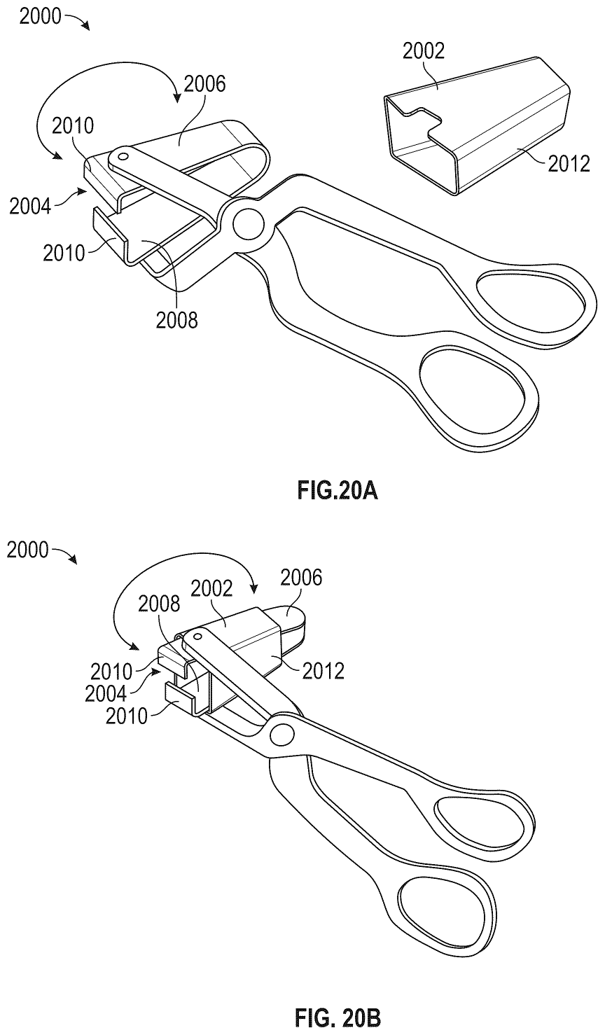

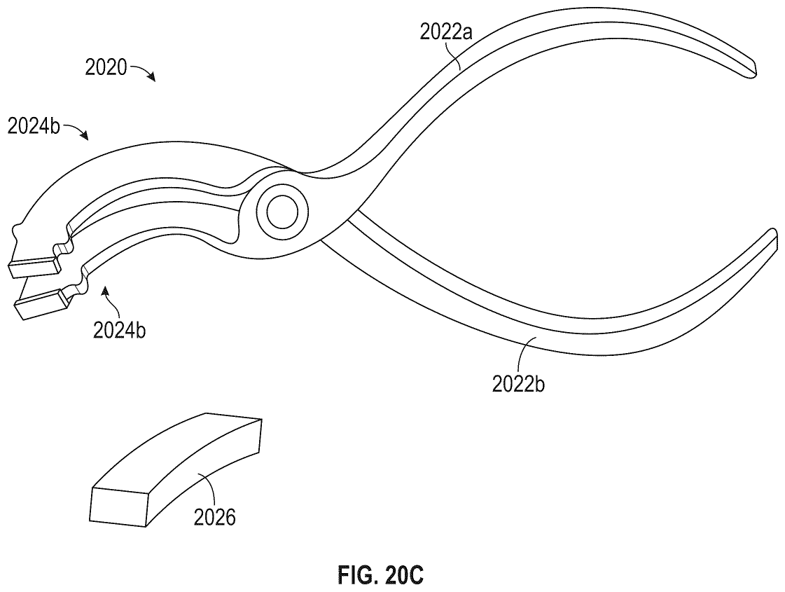

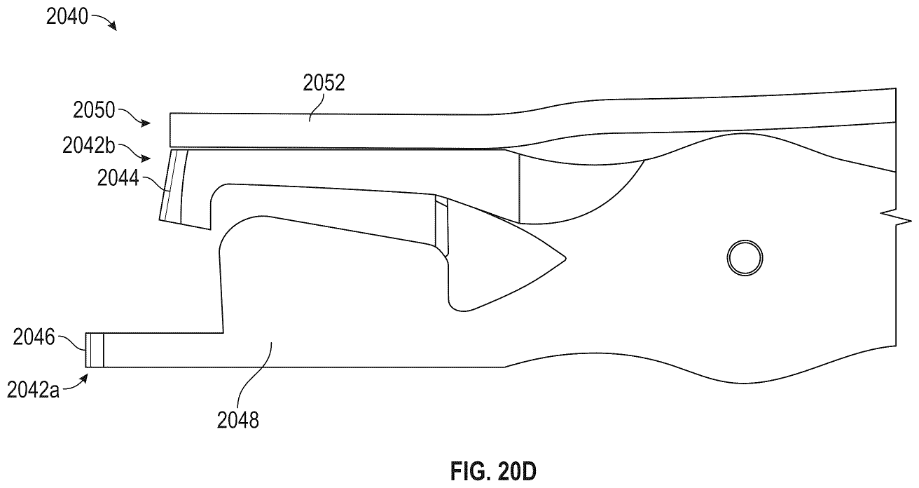

FIGS. 20A and 20B illustrate a dental auxiliary removal device 2000 with a sleeve 2002 to capture debris, in accordance with embodiments of the present technology. Specifically, FIG. 20A is a perspective view of the device 2000 and the sleeve 2002 when separated, and FIG. 20B is a perspective view of the device 2000 with the sleeve 2002.

Referring to FIGS. 20A and 20B together, the device 2000 can be generally similar to the device 1800 of FIGS. 18A-18E. For example, the device 2000 can include a distally-located rotatable assembly 2004, which may be configured similarly to the rotatable assembly 1814. The rotatable assembly 2004 includes an upper portion 2006 and a lower portion 2008 with respective scrapers 2010 which can be brought together to apply a force against the dental auxiliary to shear, scrape, cut, or otherwise separate the dental auxiliary from the tooth. Although FIGS. 20A and 20B illustrate scrapers 2010 on both the upper portion 2006 and the lower portion 2008, in other embodiments the scraper 2010 on the lower portion 2008 may be replaced with a platform for supporting the occlusal surface of the tooth during the removal process.

In the illustrated embodiment, the rotatable assembly 2004 does not include sidewalls on either the upper portion 2006 or the lower portion 2008. Instead, sidewalls 2012 are present on a sleeve 2002 that can be coupled to the rotatable assembly 2004. As shown in FIG. 20B, the sleeve 2002 can fit over the upper portion 2006 and the lower portion 2008 of the rotatable assembly 2004 such that the sidewalls 2012 are located proximal to the scrapers 2010. Accordingly, when the scrapers 2010 are closed around a dental auxiliary, the dental auxiliary and/or associated debris can be captured between the sidewalls 2012 of the sleeve 2002.

FIG. 20C illustrates a dental auxiliary removal device 2020 configured in accordance with embodiments of the present technology. The device 2020 may be generally similar to the devices described with respect to FIGS. 17A-17C and 19A-19D, except that the device 2020 includes pivotally coupled elongate members 2022a, 2022b that have curved distal ends 2024a, 2024b instead of straight distal ends. The curvature of the distal ends 2024a, 2024b can be selected to improve access to dental auxiliaries located on posterior teeth and/or other teeth that may be challenging to access from a frontal direction. Optionally, the curvature can be selected to conform to the shape of the checks, e.g., to improve patient comfort.

In the illustrated embodiment, the device 2020 does not include sidewalls, and is instead may be used with a removable sleeve 2026 including sidewalls for capturing debris (e.g., similar to the sleeve 2002 of FIGS. 20B and 20C). The sleeve 2026 may be curved to conform to the curvature of the distal ends 2024a, 2024b. In other embodiments, however, one or both distal ends 2024a, 2024b can include integrally formed sidewalls for capturing debris, such that the sleeve 2026 is optional and may be omitted.

Although FIG. 20C illustrates a device 2020 with curved distal ends 2024a, 2024b, in other embodiments, the distal ends 2024a, 2024b may be angled instead of curved. In such embodiments, the shape of the sleeve 2026 can also be angled to conform to the angle of the distal ends 2024a, 2024b.

FIG. 20D illustrates a combined dental auxiliary placement and removal device 2040, in accordance with embodiments of the present technology. The device 2040 can include any of the features of the dental auxiliary placement devices described herein and any of the features of the dental auxiliary removal devices described herein. In the illustrated embodiment, for example, the device 2040 includes a pair of elongate members that are pivotally coupled to each other, where the distal ends 2042a, 2042b of the elongate members include a scraper 2044, a platform 2046, and optional sidewalls 2048 for removing a dental auxiliary from a tooth, e.g., as discussed elsewhere herein. The device 2040 can also include an energy output region 2050 configured to output energy (e.g., light) to cure a bonding material (e.g., a light-curable dental compound) to bond a dental auxiliary to a tooth. The energy output region 2050 can be coupled to an energy source (not shown) via a conduit 2052 (e.g., tubes, tunnels, pipes, fiber optics, etc.) to allow energy to be transmitted from the energy source to the energy output region 2050.

Although FIG. 20D illustrates the energy output region 2050 and conduit 2052 as being located on the distal end 2042b of the device 2040 proximate to the scraper 2044, the energy output region 2050 and conduit 2052 can alternatively or additionally be positioned at other locations, such as on the distal end 2042a of the device 2040 proximate to the platform 2046, between the distal ends 2042a, 2042, etc. Moreover, although the energy output region 2050 and conduit 2052 are depicted as being external to the distal ends 2042a, 2042b, in other embodiments, the energy output region 2052 and conduit 2052 may be integrated into one or both distal ends 2042a, 2042b, e.g., the conduit 2052 may be an internal component that extends at least partially through an elongate member and terminates in a distal opening that serves as the energy output region 2052. Furthermore, the device 2040 may include other features not shown in FIG. 20D, such as engager arms, sensors, etc.

The devices described herein can alternatively or additionally be used for other functions besides removal of the dental auxiliaries from teeth. For example, any of the devices described herein may include measurement indicators (e.g., distance markings, angle markings) to allow a user to measure distances and/or sizes of structures in the intraoral cavity. Such measurement indicators may be located on any suitable portion of the device, such as on one or mor distal ends. As another example, the rotatable assemblies described herein may be used to support floss, interproximal reduction strips, files, probes, and/or other tools for which access to posterior and/or obstructed teeth is desired.

### C. Robotic Systems

Any of the apparatuses described herein (e.g., the dental auxiliary placement tools of Section II.A, the dental auxiliary removal tools of Section II.B) may be configured for use with, or as part of, a robotic system. For example, the apparatus may include an interface for a robotic system so that the handle(s) of the apparatus and/or any controls may be operated by the robotic system, either automatically, semiautomatically, or manually. For example, the robotic system may include one or more arms to hold and/or manipulate the apparatus. The robotic system may include one or more robotic manipulators (e.g., robotic arms, hands) that may be driven by a driver and/or drive system (e.g., electric, magnetic, pneumatic). The robotic system may include an optical sub-system to provide control and/or feedback information for guiding (e.g., steering, positioning) the apparatus, such as the devices for bonding and/or removing a dental auxiliary described herein. For example, the robotic system may include a navigation and/or operation sub-system including one or more cameras and/or one or more other sensors (e.g., position sensors, angle sensors, movement sensors, proximity sensors). The one or more cameras and/or other sensors may be configured to monitor the apparatus, including the operation of the apparatus, and/or the location of the apparatus relative to the patient (e.g., the patient's hard or soft tissue or patient's existing appliances). In some embodiments, the navigation and/or operation sub-system may be configured as an optical sub-system and may be configured to acquire live (e.g., real-time) images (e.g., video and/or photos) of the patient's dentition and/or the apparatus. The robotic system may determine the position, orientation, and/or geometries of the dental auxiliary, and may cause the apparatus to function automatically by controlling one or more actuators to manipulate the apparatus components, e.g., the body, engager(s), engager arm(s), actuating controls, etc. The robotic system may include a controller with one or more processors that may communicate with the apparatuses described herein, and may coordinate operation of the robot manipulator (e.g., robotic arm) and/or may control operation of the apparatus (e.g., tool for bonding and/or removing a dental auxiliary). The controller for the robotic system may be integrated with or (in some examples) separate from the controller of the apparatus being manipulated by the robotic system.

III. Placement of Dental Auxiliaries Proximate to Missing and/or Unerupted Teeth

In some embodiments, the dental auxiliary positioners described herein are configured to place an auxiliary on a tooth that is proximate to a missing, unerupted, or erupting tooth. The tooth may be immediately adjacent to a space left by a missing, unerupted, or erupting tooth, or there may be one or more teeth between the tooth and the space left by the missing, unerupted, or erupting tooth. A tooth that is proximate to a missing, unerupted, or erupting tooth may be more likely to move over time. For instance, the tooth may translate in a mesial-distal direction, a buccal-lingual direction, a gingival-occlusal direction, or a combination thereof. Alternatively or in addition, the tooth may rotate around a long axis of the tooth, a transverse axis of the tooth (e.g., tipping, torquing), or a combination thereof.

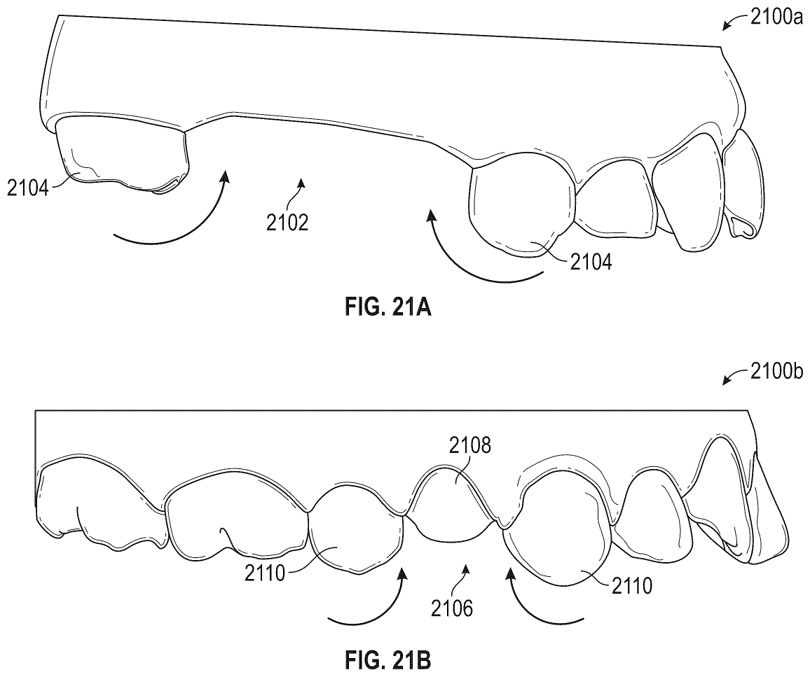

For example, FIG. 21A is a side view of a dental arch 2100a including a space 2102 due to one or more missing and/or unerupted teeth. The one or more teeth 2104 that are proximate to (e.g., immediately adjacent to) the space 2102 may have a natural tendency to move toward the space 2102, such as by tipping and/or inclining, e.g., as indicated by the arrows in FIG. 21A.

As another example, FIG. 21B is a side view of a dental arch 2100b including a space 2106 due to an erupting tooth 2108. The one or more teeth 2110 that are proximate to (e.g., immediately adjacent to) the erupting tooth 2108 may be affected by the presence of the erupting tooth 2108. For instance, the teeth 2110 may tend to move inward toward the space 2106, e.g., as indicated by the arrows in FIG. 21B. This may be because the teeth 2110 may not be sufficiently constrained in their positions by erupting tooth 2108. Alternatively, in some cases, the one or more teeth 2110 may be pushed outward and away from the space 2106 as the tooth 2108 erupts (e.g., in the opposite direction of the arrows in FIG. 21B).

Movement of a tooth may in some cases compromise the ability of a dental auxiliary positioner to accurately position an auxiliary on the tooth. For example, a dental auxiliary positioner may be designed based on a measurement of the patient's teeth (e.g., scan data) taken at an initial time. However, there is often a significant delay (e.g., four to six weeks) between the initial time at which the measurements were taken, and the subsequent time at which the positioner is actually used to place the auxiliary. The delay may be due one or more of the following: time for the clinician to submit the patient's case for treatment planning, time to generate a treatment plan for the patient's teeth, time for determining a design for a dental auxiliary positioner to place the auxiliaries on the teeth in accordance with the treatment plan, time for manufacturing the positioner, time for delivering the positioner to the clinician or patient, and/or time for scheduling the appointment to place the auxiliaries on the patient's teeth using the positioner. If the tooth has moved significantly during the delay period (e.g., due to proximity to a missing, unerupted, or erupting tooth), the positioner may no longer fit properly on the patient's teeth and/or may not locate the auxiliary at the proper position on the tooth.

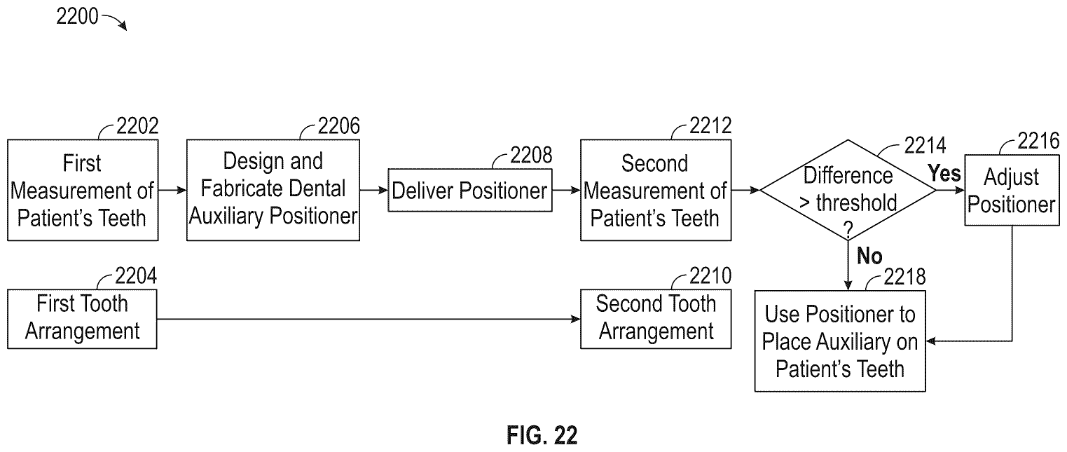

To address these and other challenges, the present technology provides methods and associated devices and systems for placing auxiliaries on teeth that are proximate to missing, unerupted, and/or erupting teeth, and/or teeth that are otherwise prone to movement. In some embodiments, for example, the present technology provides methods for placing auxiliaries on teeth by measuring the patient's teeth to detect any changes in tooth position that may have occurred, then adjusting a dental auxiliary positioner to accommodate the changes, e.g., as described in connection with FIGS. 22-28D below. The measurement can be performed digitally (e.g., based on scan data of the patient's teeth), using a physical tool (e.g., a measurement device, which can be the positioner itself or can be a different device), or suitable combinations thereof. The measurements can allow the clinician to quickly assess whether significant changes to the patient's teeth have occurred after the initial measurements, and can guide the clinician in making the appropriate adjustments to the positioner so the auxiliary can still be accurately and precisely placed at the desired location on the tooth. This approach can improve predictability of treatment, avoid treatment delays due to redesign of positioners that no longer fit properly, and/or provide improved treatment outcomes.

FIG. 22 is a schematic block diagram illustrating a workflow 2200 for placing a dental auxiliary on a tooth, in accordance with embodiments of the present technology. The workflow 2200 can be used to accurately place a dental auxiliary on the tooth, even if the tooth moves (e.g., due to proximity to a missing, unerupted, or erupting tooth).

The workflow 2200 includes obtaining a first measurement of the patient's teeth (block 2202). The first measurement can be based on any data type that provides information on the positions and orientations of some or all of the teeth, such as photographs and/or videos (as captured on, e.g., a mobile computing device such as a smartphone, or another suitable device with a camera), scan data (e.g., intraoral and/or extraoral scans), magnetic resonance imaging (MRI) data, and/or radiographic data (e.g., standard x-ray data such as bitewing x-ray data, panoramic x-ray data, cephalometric x-ray data, computed tomography (CT) data, cone-beam computed tomography (CBCT) data, fluoroscopy data). In some embodiments, for example, the first measurement is or includes scan data obtained using an intraoral scanner. The scanner can include a probe (e.g., a handheld probe) for optically capturing 3D structures (e.g., by confocal focusing of an array of light beams). Examples of scanners include, but are not limited to, the iTero® intraoral digital scanner manufactured by Align Technology, Inc. In some embodiments, for example, the first measurement may be based on 2D images obtained using a smartphone or other device with a camera.

The first measurement can be taken while the patient's teeth are in a first tooth arrangement (block 2204). In some embodiments, the first tooth arrangement is a pre-treatment tooth arrangement of the patient's teeth, such as a tooth arrangement before any dental appliances have been applied to the patient's teeth in accordance with a treatment plan. Alternatively, the first tooth arrangement can be an intermediate tooth arrangement of the patient's teeth, such as a tooth arrangement during an intermediate stage of a treatment plan in which one or more dental appliances have already been applied to the patient's teeth for a period of time. The first measurement can be obtained for all of the patient's teeth, or can be obtained for only a subset of the patient's teeth (e.g., only the teeth that will receive auxiliaries, and/or only the teeth that are likely to move).

The first measurement of the patient's teeth in the first tooth arrangement can be used to design and fabricate a dental auxiliary positioner (block 2206). The positioner can be configured to place one or more auxiliaries (e.g., attachments, buttons, power arms, brackets, wires, splints, distalizers) on one or more of the patient's teeth. The positioner can include any of the features of the embodiments of FIGS. 1A and 1B. For example, the positioner can include one or more registration elements configured to receive one or more teeth of the patient, and at least one auxiliary support coupled to a registration element and further coupled to an auxiliary in order to position the auxiliary at a predetermined location on a tooth. The positioner can also be adjustable, e.g., as described further below in connection with FIGS. 26-28D. In some embodiments, the positioner is designed based on the first measurement of the patient's teeth, such that the positioner is customized to the first tooth arrangement of the patient's teeth. For instance, the geometry and locations of the registration elements and/or auxiliary supports can correspond to the geometry and locations of the teeth in the first tooth arrangement.

The positioner can be fabricated using any suitable technique, such as a direct fabrication technique using one or more additive manufacturing processes. Examples of additive manufacturing processes include, but are not limited to, the following: (1) vat photopolymerization and similar processes in which an object is constructed from a vat or other bulk source of liquid resin, including techniques such as stercolithography (SLA), digital light processing (DLP), continuous liquid interface production (CLIP), two-photon induced photopolymerization (TPIP), and volumetric additive manufacturing; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) material extrusion, in which material is drawn though a nozzle, heated, and deposited layer-by-layer, such as fused deposition modeling (FDM) and direct ink writing (DIW); (5) powder bed fusion, including techniques such as direct metal laser sintering (DMLS), electron beam melting (EBM), selective heat sintering (SHS), selective laser melting (SLM), and selective laser sintering (SLS); (6) sheet lamination, including techniques such as laminated object manufacturing (LOM) and ultrasonic additive manufacturing (UAM); and (7) directed energy deposition, including techniques such as laser engineering net shaping, directed light fabrication, direct metal deposition, and 3D laser cladding. Optionally, the positioner can be fabricated using a combination of two or more additive manufacturing processes.

After fabrication, the positioner can be delivered to the clinician or patient (block 2208). In some situations, by the time the positioner is ready for use, the patient's teeth may be in a second tooth arrangement that differs from the first tooth arrangement (block 2210). For instance, one or more teeth may have moved (e.g., translated and/or rotated), such that at least one tooth is in a second position that is different from a first position of the tooth in the first tooth arrangement. The likelihood of tooth movement may be higher if significant time has elapsed since the first measurement of the patient's teeth, such as at least one week, two weeks, three weeks, four weeks, five weeks, six weeks, or more. The likelihood of tooth movement may also be higher for teeth that are proximate to missing, uncrupted, and/or crupting teeth, as described elsewhere herein. Significant amounts of tooth movement may interfere with proper fitting of the positioner on the patient's teeth. For instance, the positioner may not fit properly on the patient's teeth and/or may not position an auxiliary at the correct location on the target tooth if a tooth has translated by more than 1 mm, 2 mm, 3 mm, 4 mm, or 5 mm, and/or has rotated by more than 1 degree, 2 degrees, 3 degrees, 4 degrees, 5 degrees, 6 degrees, 7 degrees, 8 degrees, 9 degrees, or 10 degrees.

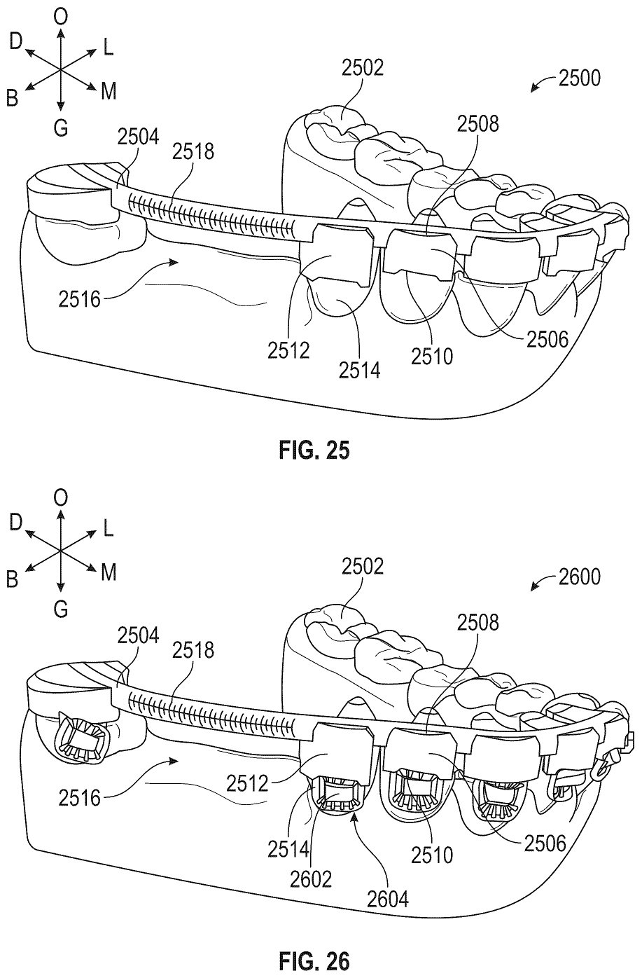

In some embodiments, the workflow 2200 includes obtaining a second measurement of the patient's teeth in the second tooth arrangement (block 2212). The second measurement can be based on any data type that provides information on the positions and orientations of the patient's teeth. For example, the second measurement may be based on photographs and/or videos (as captured on, e.g., a mobile computing device such as a smartphone, or another suitable device with a camera), scan data, MRI data, and/or radiographic data of the patient's teeth. Alternatively or in combination, the second measurement can be obtained using a physical measurement device, such as a Boley gauge, PalaMeter®, or other physical device (e.g., the devices of FIGS. 25 and 26 below). The second measurement can be obtained for all of the patient's teeth, or can be obtained for only a subset of the patient's teeth (e.g., only the teeth that will receive auxiliaries, and/or only the teeth that are likely to move).

The second measurement can be used to determine differences between the patient's teeth in the first tooth arrangement versus the second tooth arrangement. For instance, the second measurement can be used to detect whether the position of a tooth in the second tooth arrangement is different from the position of the tooth in the first tooth arrangement. The difference can be determined quantitatively (e.g., specific amounts of translation and/or rotation), qualitatively (e.g., the tooth has translated and/or rotated, without providing specific amounts; the direction of translation and/or rotation), or suitable combinations thereof.

In some embodiments, the workflow includes determining whether the difference between the patient's teeth in the first tooth arrangement versus the second tooth arrangement exceeds a threshold difference (block 2214). The threshold difference can represent a difference in translation and/or rotation that, if exceeded, will likely interfere with proper fitting of the dental auxiliary positioner on the patient's teeth. The threshold difference may be the same for all of the patient's teeth, or there may be different threshold differences for different teeth. For instance, the threshold difference may be smaller for teeth that are more important for proper fitting of the positioner, for teeth that will receive auxiliaries, and/or for teeth proximate to the teeth that will receive auxiliaries. Conversely, the threshold difference may be larger for teeth that are less important for proper fit, for teeth that will not receive auxiliaries, and/or for teeth away from the teeth that will receive auxiliaries.

If the difference is greater than the threshold difference, the positioner can be adjusted to accommodate any differences between the patient's teeth between the first and second tooth arrangements (block 2216). For instance, the positioner can be designed, fabricated, and/or provided in a first configuration that corresponds to the first tooth arrangement of the patient's teeth, and can subsequently be adjusted to a second, different configuration that corresponds to the second tooth arrangement of the patient's teeth. The adjustments can be made, for example, by adjusting a registration element of the positioner, adjusting an auxiliary support of the positioner, or suitable combinations thereof. Examples of positioners with adjustable components are described in greater detail, for example, in connection with FIGS. 26-28D below.

The adjusted positioner can then be used to place the auxiliary on the patient's teeth (block 2218). For example, the positioner can be placed on the patient's teeth to position the auxiliary at a predetermined location on a surface of a tooth, the auxiliary can be mounted to the tooth via an adhesive (e.g., a UV-curable adhesive), and the positioner can then be separated from the auxiliary and removed from the patient's teeth while the auxiliary remains in place on the tooth.

In some embodiments, the second measurement of the teeth, adjustment to the positioner, and placement of the auxiliary are performed on the same day (e.g., at the same appointment). In other embodiments, the second measurement, adjustment, and placement can be performed at different times (e.g., on different days and/or at different appointments), as long as the amount of time elapsed between the second measurement and the placement of the auxiliary is sufficiently short to avoid significant changes to the patient's teeth. For instance, the time elapsed between the second measurement and the placement of the auxiliary may be no more than three days, two days, or one day.

Alternatively, if the difference is less than or equal to the threshold difference (block 2214), the positioner can be used to place the auxiliary on the patient's teeth (block 2218), without making any adjustments to the positioner.

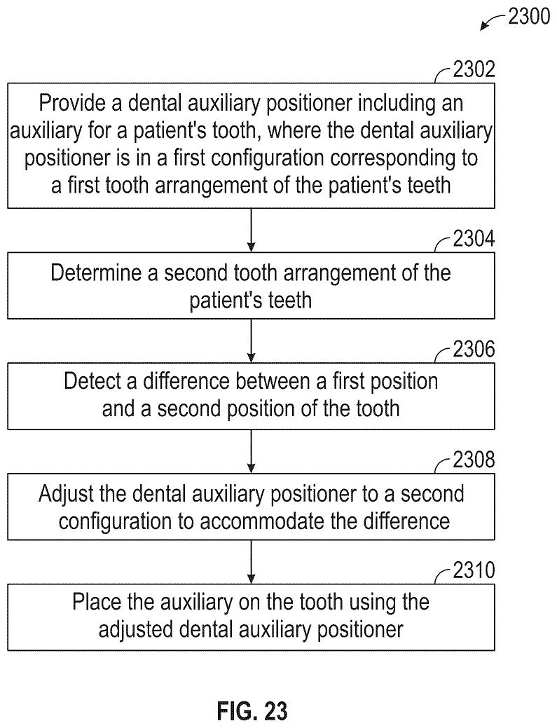

FIG. 23 is a flow diagram illustrating a method 2300 for placing a dental auxiliary on a tooth, in accordance with embodiments of the present technology. For instance, the method 2300 can be used to place an auxiliary on a tooth that is prone to movement, such as a tooth that is proximate to a space associated with a missing, unerupted, or erupting tooth. The method 2300 can be combined with and/or performed as part of the workflow 2200 of FIG. 22. Some or all of the processes of the method 2300 can be performed manually (e.g., by a clinician or other healthcare provider), and/or some or all of the processes of the method 2300 can be implemented as computer-readable instructions (e.g., program code) that are configured to be executed by one or more processors of a computing device.

The method 2300 can begin at block 2302 with providing a dental auxiliary positioner including an auxiliary for a patient's tooth. The auxiliary can be an attachment, button, power arm, bracket, wire, split, distalizer, or other device that is configured to be affixed to the tooth in order to engage a dental appliance (e.g., an aligner, palatal expander), e.g., to apply force to the tooth, in accordance with a treatment plan. The dental auxiliary positioner can be configured to place the auxiliary at a predetermined location on the tooth. For example, the dental auxiliary positioner can include one or more registration elements configured to receive one or more teeth of the patient, and at least one auxiliary support coupled to the auxiliary, e.g., as described above in connection with FIGS. 1A and 1B.

In some embodiments, the dental auxiliary positioner is provided in a first configuration corresponding to a first tooth arrangement of the patient's teeth (e.g., a pre-treatment tooth arrangement or an intermediate tooth arrangement). For instance, the dental auxiliary positioner can be designed to fit the first tooth arrangement of the patient's teeth, such that the locations and geometry of the registration element(s) and auxiliary support(s) of the positioner correspond to the locations and geometry of the patient's teeth in the first tooth arrangement. The dental auxiliary positioner can be designed based on a first measurement of the patient's teeth that is obtained while the teeth are in the first tooth arrangement. The first measurement can be based on scan data of the first tooth arrangement of the patient's teeth and/or any of the other data types described herein.

At block 2304, the method 2300 can include determining a second tooth arrangement of the patient's teeth, and at block 2306, the method 2300 can include detecting a difference between a first position of the tooth in the first tooth arrangement and a second position of the tooth in the second tooth arrangement. The second tooth arrangement can correspond to the patient's teeth at a time subsequent to the first tooth arrangement, such as at least one week, two weeks, three weeks, four weeks, five weeks, six weeks, or more after the first tooth arrangement. The second tooth arrangement may be different than the first tooth arrangement of the patient's teeth due to movements of one or more teeth during the intervening time period. For example, the tooth that is intended to receive the auxiliary may have been in a first position in the first tooth arrangement, but may now be in a second position in the second tooth arrangement that differs from the first position. The tooth movement can be caused, for example, by proximity of the tooth to a missing, unerupted, or erupting tooth.

The second tooth arrangement can be determined in various ways, such as by obtaining a second measurement of the patient's teeth. For example, the second tooth arrangement can be determined by obtaining digital data of the second tooth arrangement of the patient's teeth, such as scan data or image data (e.g., from a smartphone or other camera device). The digital data (e.g., scan data) of the second tooth arrangement can be compared to digital data of the first tooth arrangement to detect any changes in tooth position that may have occurred between the first and second tooth arrangements. In some embodiments, the digital data of the first and second tooth arrangements is received and/or accessed by a computing device (e.g., a laptop, tablet, workstation, personal computer, mobile device) that implements a software application configured to compare the digital data to identify any changes in the positions of the teeth in the first and second tooth arrangements. The comparison can involve matching each tooth in the digital data of the first tooth arrangement to the corresponding tooth in the digital data of the second tooth arrangement, then calculating a deviation between the positions of each tooth in the second tooth arrangement versus the first tooth arrangement. The output of the calculation can include, for each tooth, at least one distance value indicating an amount of translational movement in one or more directions, and/or at least one angular value indicating an amount of rotational movement in one or more directions. The comparison can be performed for all of the patient's teeth, or can be performed for only a subset of the patient's teeth (e.g., only the teeth that will receive auxiliaries, and/or only the teeth that are likely to move).

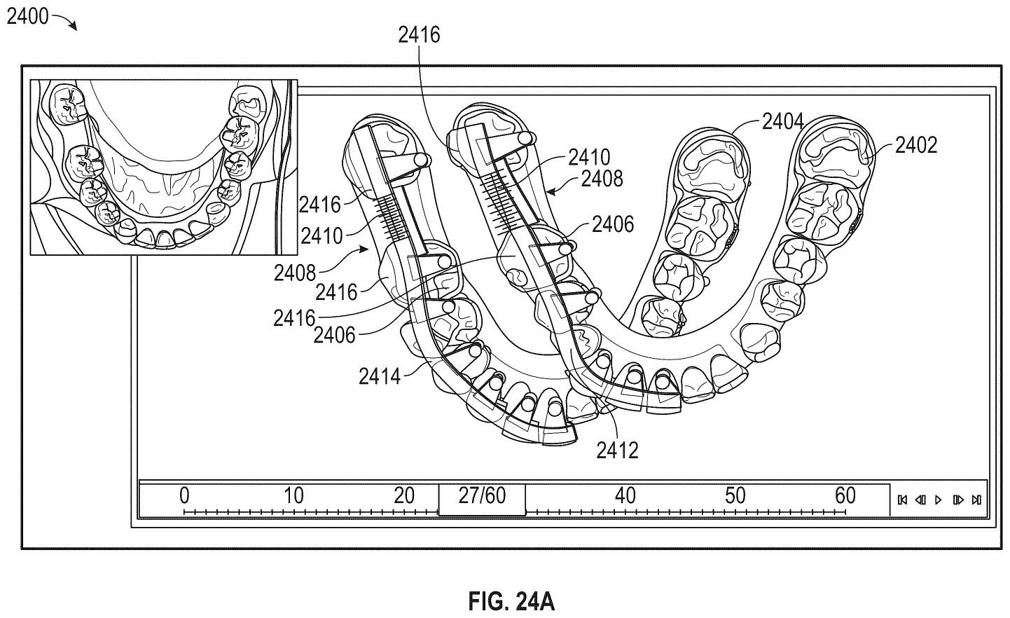

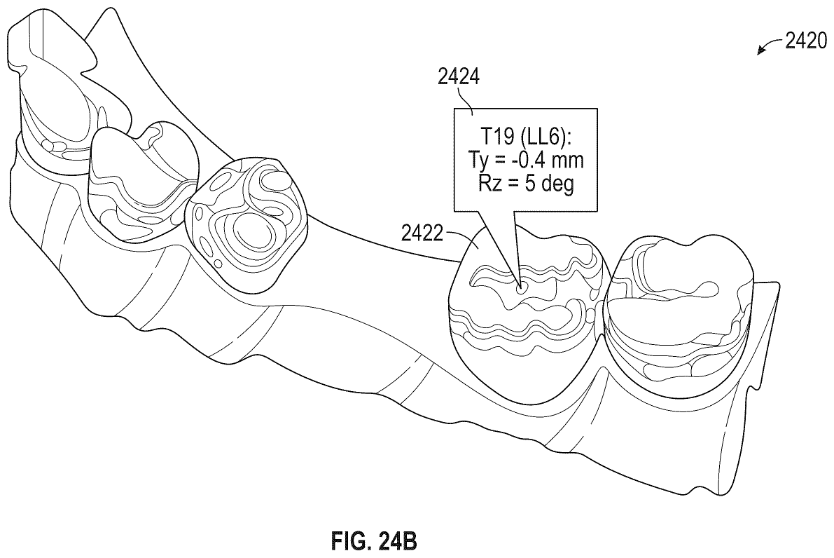

The software application can output the comparison results to a display for viewing by a user (e.g., a clinician), so the user can evaluate the locations and extent of any tooth movements that have occurred. The comparison results can be displayed in any suitable format, such as graphically (e.g., 3D models, 2D images, animations), textually (e.g., numerical values, textual description), or suitable combinations thereof. Optionally, the software application can also output other relevant information, such as alerts (e.g., an alert indicating that the detected tooth movement exceeds a threshold value, an alert indicating that adjustments to the positioner are needed or suggested, an alert indicating that a new positioner is needed), recommendations (e.g., instructions for adjusting the positioner to accommodate tooth movements), etc. Representative examples of information output by a software application for detecting tooth movement are described, e.g., in connection with FIGS. 24A and 24B below.

Alternatively or in combination, the second tooth arrangement can be determined using a physical measurement device. In some embodiments, the physical measurement device is an adjustable device including at least one component that can be moved to different positions corresponding to the range of possible positions for the tooth. The device can further include one or more measurement indicators that can be read by a user (e.g., the clinician) to assess the direction and/or amount of movement of the component, and thus the direction and/or amount of movement of the tooth relative to the first tooth arrangement. Optionally, the device can be customized to the particular patient (e.g., the device can be configured to fit specifically on the patient's teeth), rather than being a generic, off-the-shelf tool. Representative examples of devices for measuring tooth movement are described, e.g., in connection with FIGS. 25 and 26 below.

At block 2308, the method 2300 can continue with adjusting the dental auxiliary positioner to a second configuration to accommodate the difference between the first and second positions of the tooth. The adjustments can be made such that when the adjusted dental auxiliary positioner is worn on the patient's teeth in the second tooth arrangement (or in a tooth arrangement similar to the second tooth arrangement, such as a tooth arrangement close in time to the second tooth arrangement), the auxiliary is positioned at or near the predetermined location on the target tooth.

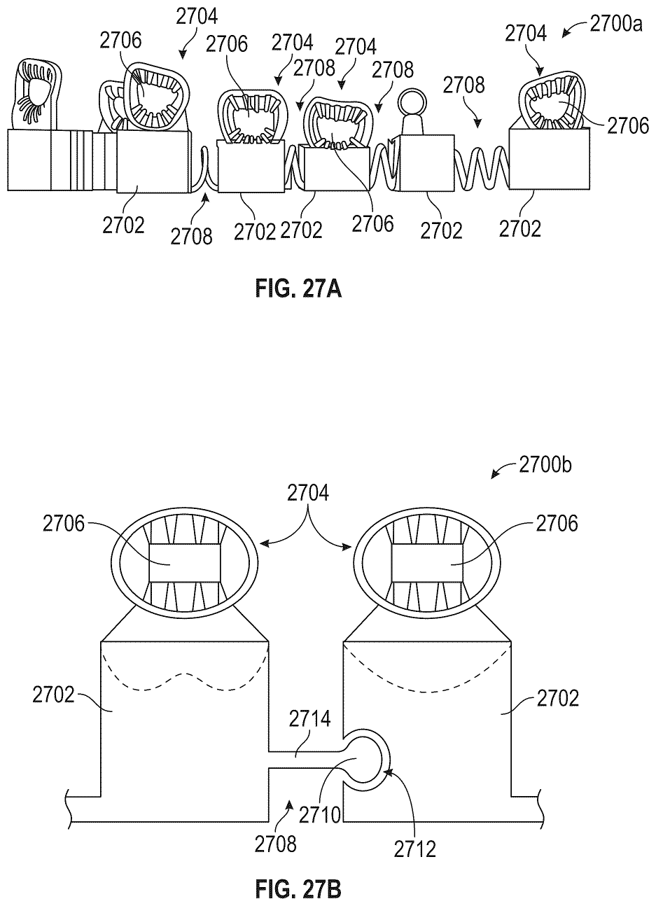

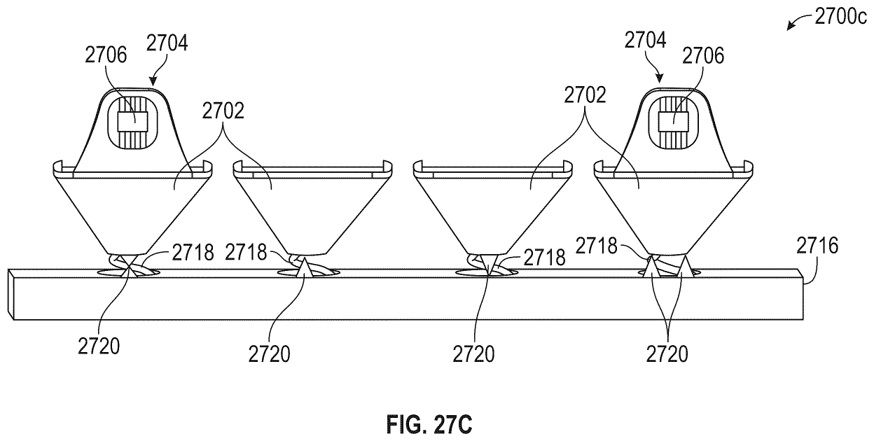

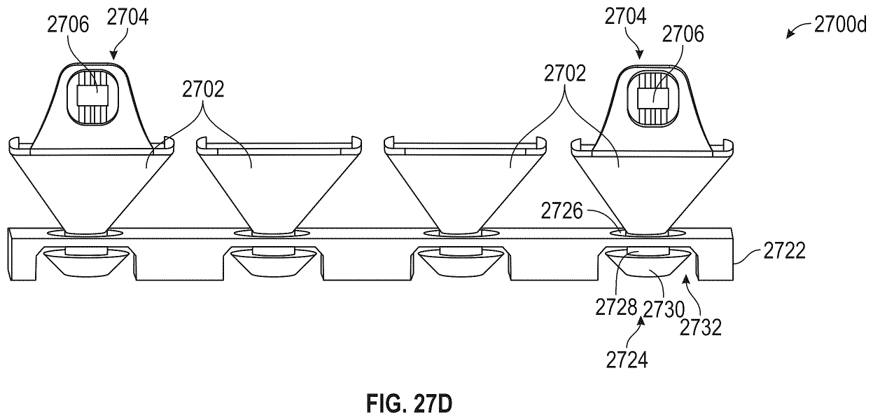

The adjustments can be made in various ways. For instance, in embodiments where the dental auxiliary positioner includes one or more registration elements, and at least one auxiliary support coupled to the auxiliary (e.g., as described above in connection with FIGS. 1A and 1B), the process of block 2308 can involve adjusting a registration element, adjusting an auxiliary support, or a combination thereof. For example, a registration element can be adjusted by moving the registration element relative to another portion of the dental auxiliary positioner (e.g., relative to another registration element that is proximate to the registration element). In such embodiments, the registration element can be coupled to the other portion of the dental auxiliary positioner via a coupling mechanism that permits relative movement of the registration element, e.g., in one, two, or three degrees of freedom in translation and/or one, two, or three degrees of freedom in rotation. For instance, the coupling mechanism can allow relative translation of the registration element in a mesial-distal direction, a buccal-lingual direction, and/or a gingival-occlusal direction; and/or relative rotation around a mesial-distal axis, a buccal-lingual axis, and/or a gingival-occlusal axis. The coupling mechanism can include flexible connectors (e.g., springs, elastic and/or deformable materials, structures with holes, channels, etc., formed therein to provide flexibility), rotatable joints (e.g., hinges, ball-and-socket joints), slidable joints, interlocking links, and/or suitable combinations thereof. Representative examples of coupling mechanisms are described, e.g., in connection with FIGS. 27A-27D below.

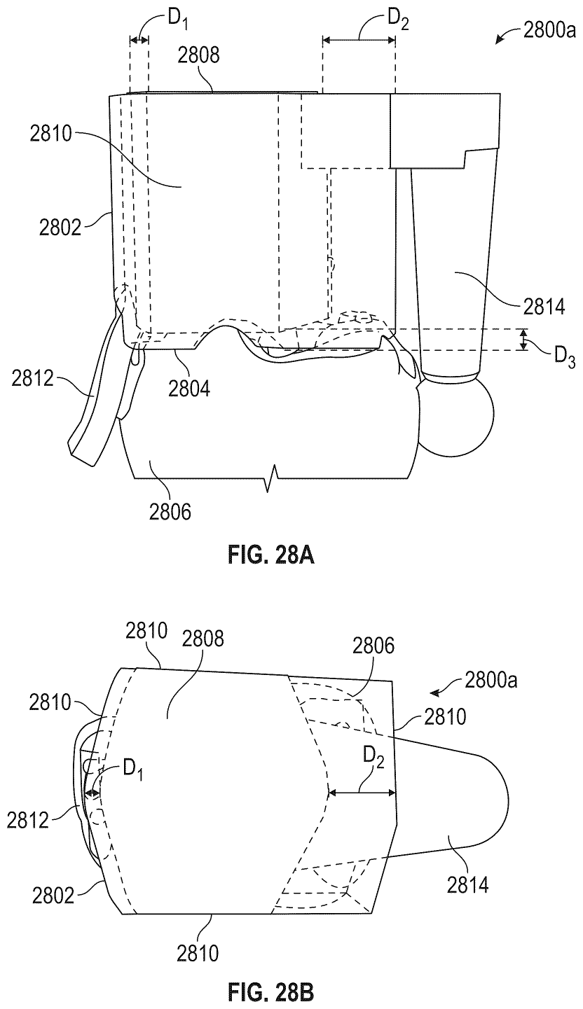

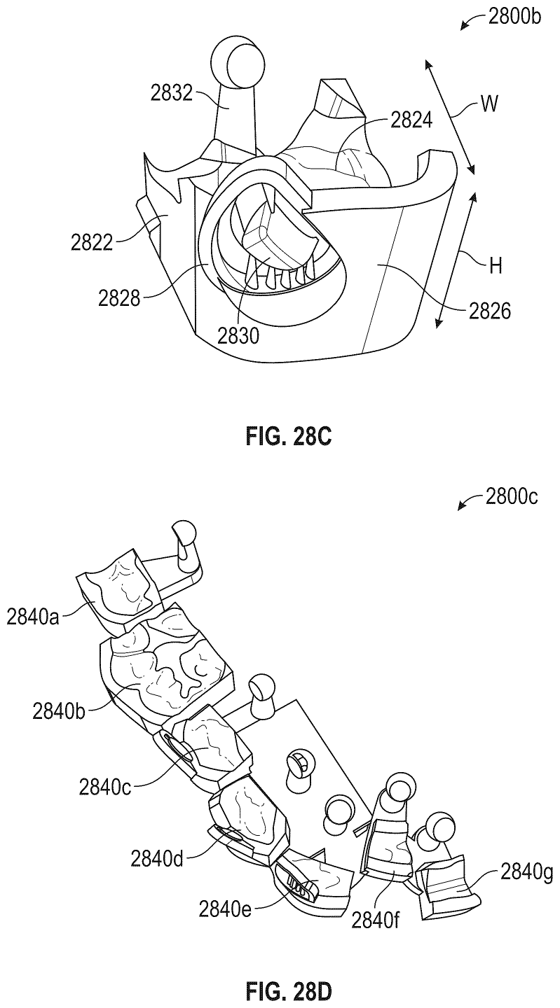

Alternatively or in combination, the registration element can be adjusted by separating the registration element from another portion of the dental auxiliary positioner. Separation can be performed, for example, by breaking a connection between the registration element and the other portion, or by decoupling a releasable coupling between the registration element and the other portion (e.g., without breaking any components of the positioner). In such embodiments, the registration element and/or a portion of the dental auxiliary positioner containing the registration element can include features configured to maintain stable coupling to the patient's teeth even when separated from the rest of the positioner. Representative examples of such stabilizing features are described, e.g., in connection with FIGS. 28A-28D below. The dental auxiliary positioner may be fabricated to facilitate the separation, for example, by having frangible features (e.g., perforations, thinner portions) along points of connection that are configured to be severable.

An auxiliary support can be adjusted by moving the auxiliary support relative to another portion of the dental auxiliary positioner (e.g., relative to a registration element that is coupled to the auxiliary support). In such embodiments, the auxiliary support can be coupled to the other portion of the dental auxiliary positioner via a coupling mechanism that permits relative movement of the auxiliary support, e.g., in one, two, or three degrees of freedom in translation and/or one, two, or three degrees of freedom in rotation. For instance, the coupling mechanism can allow relative translation of the auxiliary support in a mesial-distal direction, a buccal-lingual direction, and/or a gingival-occlusal direction; and/or relative rotation around a mesial-distal axis, a buccal-lingual axis, and/or a gingival-occlusal axis. The coupling mechanism can include flexible connectors (e.g., springs, elastic and/or deformable materials, structures with holes, channels, etc., formed therein to provide flexibility), rotatable joints (e.g., hinges, ball-and-socket joints), slidable joints, interlocking links, and/or suitable combinations thereof.

In some embodiments, the second tooth arrangement of the patient's teeth is determined using a physical measurement device, (e.g., the device 2500 described below in connection with FIG. 25), and the dental auxiliary positioner is a different device. In other embodiments, however, the physical measurement device can also be the dental auxiliary positioner (e.g., the device 2600 described below in connection with FIG. 26). In such embodiments, the determination of the second tooth arrangement and the adjustment to the dental auxiliary positioner can be performed concurrently.

At block 2310, the method 2300 can include placing the auxiliary on the tooth using the adjusted dental auxiliary positioner. As described elsewhere herein, the placement process can include mounting the auxiliary to the tooth using an adhesive, separating the auxiliary from the dental auxiliary positioner, then removing the dental auxiliary positioner from the patient's teeth while leaving the auxiliary bonded in place on the tooth.

The method 2300 illustrated in FIG. 23 can be modified in many different ways. For example, some of the processes of the method 400 can be omitted, such as the processes of block 2302 and/or 2306. For example, in some embodiments, a clinician may position the dental auxiliary positioner as provided and may simply adjust the dental auxiliary position as needed without detecting a difference between the first and second positions, or without even referring to the first position (thus omitting block 2306). The method 2300 can include additional processes not shown in FIG. 23, and/or the order of the processes shown in FIG. 23 can be varied as desired. Moreover, although the above processes of the method 400 are described with respect to a single auxiliary on a single tooth, the method 2300 can be used to detect changes in the positions of multiple teeth and/or to determine adjustments to a dental auxiliary positioner configured to place multiple auxiliaries on the teeth.

FIG. 24A illustrates a user interface (“UI”) 2400 for displaying a comparison between a first tooth arrangement and a second tooth arrangement of a patient's teeth, in accordance with embodiments of the present technology. The UI 2400 can be displayed on an output device to facilitate determination of changes in tooth position between the first and second tooth arrangements, e.g., as part of the processes of blocks 2304 and/or 2306 of the method 2300 of FIG. 23. As shown in FIG. 24A, the UI 2400 can display a first dental representation 2402 representing the first tooth arrangement of the patient's teeth, and a second dental representation 2404 representing the second tooth arrangement of the patient's teeth. As used herein, the term “dental representation” refers to a two-dimensional image or a three-dimensional model representing all or a subset of a patient's teeth. The first dental representation 2402 can be generated based on a first measurement (e.g., first scan data) of the first tooth arrangement, and the second dental representation 2404 can be generated based on a second measurement (e.g., second scan data) of the second tooth arrangement.

The first dental representation 2402 and the second dental representation 2404 can be displayed on the UI 2400 so a user (e.g., the clinician) can visually assess the differences between the first and second tooth arrangements. For instance, the first dental representation 2402 can be overlaid with the second dental representation 2404, the first dental representation 2402 can be displayed side-by-side with the second dental representation 2404, the user can toggle between displaying the first dental representation 2402 and the second dental representation 2404, the UI 2400 can show an animation transitioning between the first dental representation 2402 and the second dental representation 2404, etc.

The UI 2400 can also display one or more indicators showing the location and/or amount of any differences in tooth position between the first and second tooth arrangements. In the illustrated embodiment, for example, a tooth 2406 proximate to a space 2408 left by a missing, unerupted, and/or erupting tooth has moved to close the space 2408, and the UI 2400 displays a virtual gauge 2410 showing that the size of the space 2408 in the second dental representation 2404 is smaller than the size of the space 2408 in the first dental representation 2402. Alternatively or in combination, the UI 2400 can use other types of indicators to denote changes in tooth position, such as textual and/or numerical annotations (e.g., a numerical value in millimeters to represent a difference in distance, an angle or vector value to represent a difference in orientation and/or position), markers, coloring, etc. In some examples, UI 2400 may display an overlay of the two dental representations, with the differences being highlighted visually using a suitable indicator (e.g., in a different color). In some examples, UI 2400 may display a simplified view that only depicts a subregion of the dental representations that are clinically relevant for auxiliary placement. For example, only a subregion of the dental representations 2402 and 2404 that includes and/or is adjacent to the space 2408 may be depicted.

Optionally, the UI 2400 can also display a first positioner representation 2412 together with the first dental representation 2402, and/or a second positioner representation 2414 together with the second dental representation 2404. As used herein, the term “positioner representation” refers to a two-dimensional image or a three-dimensional model representing all or a subregion of a dental auxiliary positioner. The first positioner representation 2412 can represent a first configuration of a dental auxiliary positioner to place one or more auxiliaries on the teeth in the first tooth arrangement, and the second positioner representation 2414 can represent a second configuration of the dental auxiliary positioner to place the auxiliaries on the teeth in the second tooth arrangement. As shown in FIG. 24A, the second positioner representation 2414 can be adjusted relative to the first positioner representation 2412 to accommodate the changes in the position of the tooth 2406. For instance, the distance between registration elements 2416 flanking the space 2408 can be shortened in the second configuration, to conform to the smaller size of the space 2408. Alternatively or in combination, other types of adjustments to the positioner can be made, as discussed elsewhere herein. The first positioner representation 2412 and/or second positioner representation 2414 can be displayed to the user to provide visual guidance for adjusting the physical dental auxiliary positioner to accommodate changes in tooth position. In other embodiments, however, the first positioner representation 2412 and/or the second positioner representation 2414 are optional and can be omitted, and/or the guidance can be provided in other formats (e.g., as textual instructions). For example, a textual instruction may instruct a clinician to adjust the dental auxiliary positioner such that an auxiliary is moved in a specified direction (e.g., distally, mesially) by a specified amount (e.g., 0.5 millimeters).

FIG. 24B illustrates a digital dental representation 2420 (e.g., a 2D image, a 3D model) for displaying a comparison between a first tooth arrangement and a second tooth arrangement of a patient's teeth, in accordance with embodiments of the present technology. The dental representation 2420 can be displayed as part of the UI 2400, e.g., alternatively or in addition to the first dental representation 2402 and/or the second dental representation 2404. The dental representation 2420 can be a representation of a tooth arrangement of the patient's teeth (e.g., the first tooth arrangement or the second tooth arrangement) with an overlaid heat map 2422. The heat map 2422 can include a plurality of different colors representing the deviation between the tooth surfaces in the first tooth arrangement versus the second tooth arrangement. For instance, the heat map 2422 can have a first set of colors representing positive distances between a tooth surface in the first tooth arrangement versus the second tooth arrangement, a second set of colors representing negative distances between a tooth surface in the first tooth arrangement versus the second tooth arrangement, and a third color representing zero distance between the tooth surface in the first tooth arrangement versus the second tooth arrangement. Optionally, an indicator such as a tooltip 2424 can be displayed to provide additional information on specific teeth, such as the identifier for the tooth, the direction and/or amount of translational movement, and/or the direction and/or amount of rotational movement. Thus, a user (e.g., the clinician) can view the dental representation 2420 to evaluate the locations and/or amounts of any changes in tooth position that have occurred.

FIG. 25 is a perspective view of a device 2500 for measuring movement of a patient's teeth 2502, in accordance with embodiments of the present technology. The device 2500 can be used to determine changes in tooth position between first and second tooth arrangements of the teeth 2502, e.g., as part of the processes of blocks 2304 and/or 2306 of the method 2300 of FIG. 23. The device 2500 includes a base 2504, and one or more registration elements 2506 coupled to the base 2504. Each registration element 2506 can be configured to receive a respective tooth 2502 of the patient. A registration element 2506 may receive a tooth 2502 by, for example, receiving and/or extending over only a portion of the tooth, such as the crown of the tooth, a portion of the tooth proximate to the crown, a buccal portion of an occlusal surface of the tooth, etc. Although the device 2500 is illustrated as having six registration elements 2506, in other embodiments, the device 2500 can include a different number of registration elements 2506, such as one, two, three, four, five, eight, ten, 16, 20, or 32 registration elements 2506. Moreover, although the device 2500 is illustrated as receiving only a subset of the patient's teeth 2502, in other embodiments, the device 2500 can instead receive all of the patient's teeth 2502.

In the illustrated embodiment, each registration element 2506 includes a first portion 2508 coupled to the base 2504, and a second portion 2510 configured to receive and engage a corresponding tooth 2502. For instance, the first portion 2508 can be a first side of the registration element 2506 that is located away from the teeth 2502, and the second portion 2510 can be a second, opposite side of the registration element 2506 that is located proximate to the teeth 2502. The first portion 2508 can be coupled to the base 2504 using any suitable attachment technique, such as adhesives, mechanical fixation (e.g., interference fit, snap fit, interlocking features), fasteners, etc. Optionally, the first portion 2508 may be integrally formed with the base 2504. The coupling between the first portion 2508 and the base 2504 may be a rigid coupling or a movable coupling. The second portion 2510 of the registration element 2506 can include a cavity formed therein to define an inner surface that engages a corresponding surface of the received tooth (e.g., the occlusal, buccal, and/or lingual surface of the tooth). For instance, the topography of the inner surface can be complementary to the topography of the corresponding surface of the tooth.

The base 2504 can join the registration elements 2506 together to form a unitary device body. In the illustrated embodiment, the base 2504 is an elongate shaft (e.g., a rod, bar, bridge, or other connector) that extends along at least a portion of or the entirety of the patient's arch. In other embodiments, however, the base 2504 can be configured differently, e.g., the base 2504 can be a plurality of discrete connectors extending between neighboring pairs of registration elements 2506, rather than being a single component that connects to each of the registration elements 2506.

In some embodiments, the device 2500 includes at least one adjustable registration element (“adjustable element 2512”) that is movable relative to the base 2504, e.g., along at least one translational direction and/or at least one rotational direction. The adjustable element 2512 can be configured to a receive a tooth 2514 that is susceptible to movement, e.g., the tooth 2514 can be proximate to a space 2516 associated with a missing, unerupted, or erupting tooth. The adjustable element 2512 can be coupled to the base 2504 in a manner that allows the adjustable element 2512 to move to accommodate a range of different possible positions for the tooth 2514. For instance, the adjustable element 2512 can be movable relative to the base 2504 in one, two, or three degrees of freedom in translation and/or one, two, or three degrees of freedom in rotation. Alternatively or in combination, the adjustable element 2512 can be translatable relative to the base 2504 along a mesial-distal axis MD, a buccal-lingual axis BL, and/or a gingival-occlusal axis GO; and/or can be rotatable relative to the base 2504 around the mesial-distal axis MD, buccal-lingual axis BL, and/or gingival-occlusal axis GO. The adjustable element 2512 can be coupled to the base 2504 via any suitable coupling mechanism that permits movement in the desired directions, such as a slidable and/or rotatable coupling. In the illustrated embodiment, for example, the adjustable element 2512 includes a slot oriented in a mesial-distal direction that receives a portion of the base 2504, such that the adjustable element 2512 can slide in a mesial-distal direction along the base 2504.

Optionally, the base 2504 can also be adjustable to accommodate tooth movement. For instance, the base 2504 can be made out of a flexible and/or deformable material that can be bent, expanded, compressed, or otherwise reshaped to conform to changes in the tooth arrangement of the patient's teeth 2502. Alternatively or in combination, the base 2504 can have a structure that allows for bending, expansion, compression, etc., to accommodate such changes. In some embodiments, for example, the base 2504 can be made of a plurality of discrete segments that are connected to each other via a coupling mechanism that permits translational and/or rotational movement of the segments relative to each other. The base 2504 can be adjustable only at the portions proximate to the adjustable element 2512, or the entirety of the base 2504 can be adjustable. In other embodiments, however, the base 2504 can be a nonadjustable rigid structure having a fixed geometry.

The base 2504 can include a plurality of measurement indicators 2518 proximate to the adjustable element 2512 so the movement distance of the adjustable element 2512 can be measured. In the illustrated embodiment, for example, the indicators 618 are a series of distance markings extending along the length of the base 2504 in a mesial-distal direction near the space 2516 in the patient's arch. Accordingly, the indicators 2518 can be used to measure the amount of movement of the adjustable element 2512 along the mesial-distal direction, and thus, the movement of the tooth 2514 along the mesial-distal direction. The configuration of the indicators 2518 can be varied as desired, depending on the possible directions of movement of the adjustable element 2512. For example, the base 2504 can alternatively or additionally include distance markings in a buccal-lingual direction and/or a gingival-occlusal direction, the base 2504 can alternatively or additionally include angle markings to measure rotation of the adjustable element 2512 relative to the base 2504, etc.

In some embodiments, the device 2500 is provided with the adjustable element 2512 in an initial position corresponding to the first position of the tooth 2514 in the first tooth arrangement of the patient's teeth 2502, and the adjustable element 2512 is movable to an adjusted position to accommodate the second position of the tooth 2514 in the second tooth arrangement of the patient's teeth 2502. Accordingly, the measurement indicators 2518 can be configured to measure the amount and/or direction of movement of the adjustable element 2512 away from the initial position, thereby allowing the difference between the initial and adjusted positions to be measured. For instance, a nonzero movement distance (e.g., a positive or negative movement distance) of the adjustable element 2512 may indicate that the second position of the tooth 2514 is different from the first position of the tooth 2514, whereas a zero or substantially zero movement distance of the adjustable element 2512 may mean that the second position of the tooth 2514 is the same or substantially the same as the first position of the tooth 2514.

The indicators 2518 can be viewed by a user (e.g., the clinician) to determine the appropriate adjustments to be made to a dental auxiliary positioner to accommodate the movement of the tooth 2514, as described further below. In some embodiments, for example, the user can view the indicators 2518 to determine quantitative values for translational and/or rotational adjustments to fit the tooth 2514. Alternatively, the user can view the indicators 2518 to assess the general direction and/or extent of adjustments to be made, without determining quantitative values for translational and/or rotational adjustments.

Although FIG. 25 depicts a single adjustable element 2512, in other embodiments, the device 2500 can include a plurality of adjustable elements 2512 (e.g., two, three, four, five, eight, ten, 16, 20, or 32 adjustable elements 2512). The adjustable elements 2512 can correspond to teeth that are prone to movement (e.g., teeth proximate to the space 2516), teeth that are intended to receive auxiliaries, and/or any other suitable teeth. In some embodiments, all of the registration elements 2506 are adjustable elements 2512 that are movably coupled to the base 2504. Alternatively, only some of the registration elements 2506 are adjustable elements 2512, such that the remaining registration elements 2506 are fixedly coupled to the base 2504. In embodiments where the device 2500 includes a plurality of adjustable elements 2512, the base 2504 can include measurement indicators 2518 proximate to some or all of the adjustable elements 2512. This approach allows a single device 2500 to be used to measure the positions of multiple teeth.

FIG. 26 is a perspective view of a device 2600 for measuring movement of a patient's teeth 2502, in accordance with embodiments of the present technology. The device 2600 can be used to determine changes in tooth position between first and second tooth arrangements of the teeth 2502, e.g., as part of the processes of blocks 2304 and/or 2306 of the method 2300 of FIG. 23. The device 2600 can be generally similar to the device 2500 of FIG. 25 and can include all of the features of the device 2500, except that the device 2600 can also be used as a dental auxiliary positioner to place one or more auxiliaries on the teeth 2502. In the illustrated embodiment, for example, the adjustable element 2512 is coupled to an auxiliary support 2604 for an auxiliary 2602 on the tooth 2514. The auxiliary support 2604 can be generally similar to the other embodiments described herein (e.g., the auxiliary supports 110 of FIGS. 1A and 1B). The auxiliary support 2604 can be rigidly coupled to the adjustable element 2512 so that the auxiliary support 2604 and auxiliary 2602 remain in a fixed spatial relationship with the adjustable element 2512. Alternatively, the auxiliary support 2604 can be movably coupled to the adjustable element 2512 so that the auxiliary support 2604 and the auxiliary 2602 are movable (e.g., translatable and/or rotatable) relative to the adjustable element 2512.

The adjustable element 2512 including the auxiliary support 2604 can be coupled to the base 2504 in a manner that allows the adjustable element 2512 to move (e.g., rotate and/or translate) to accommodate a range of different possible positions for the tooth 2514, e.g., as discussed above in connection with FIG. 25. In some embodiments, the device 2600 is provided with the adjustable element 2512 and auxiliary support 2604 in an initial position corresponding to the first position of the tooth 2514 in the first tooth arrangement of the patient's teeth 2502, and the adjustable element 2512 and auxiliary support 2604 are movable to an adjusted position to accommodate the second position of the tooth 2514 in the second tooth arrangement of the patient's teeth 2502. Thus, the movement of the adjustable element 2512 and the auxiliary support 2604 can be used not only to detect changes in tooth position (e.g., based on the measurement indicators 2518 proximate to the adjustable element 2512), but can also be used to adjust the device 2600 so the auxiliary 2602 is positioned at the appropriate location on the tooth 2514 for bonding, as described in greater detail below. Optionally, the base 2504 can also be adjustable to accommodate changes in tooth position.

Optionally, as shown in FIG. 26, some or all of the other registration elements 2506 of the device 2600 (which may or may not also be adjustable elements 2512) can also include respective auxiliary supports 2604 for placing auxiliaries 2602 on the corresponding received teeth 2502. In some embodiments, the device 2600 is used to place auxiliaries 2602 on teeth that are susceptible to movement (e.g., using adjustable elements 2512) and on teeth that are not expected to move substantially (e.g., using fixed registration elements 2506). Alternatively, the device 2600 can be used to place auxiliaries 2602 only on teeth 2502 that are susceptible to movement, while a separate dental auxiliary positioner can be used to place auxiliaries on the teeth 2502 that are not expected to move substantially.

FIGS. 27A-27D illustrate representative examples of adjustable dental auxiliary positioners for placing auxiliaries on a patient's teeth, in accordance with embodiments of the present technology. The embodiments of FIGS. 27A-27D can be used in any of the methods described herein, such as the method 2300 of FIG. 23. Moreover, any of the features of the embodiments of FIGS. 27A-27D can be combined with each other and/or with any of the other embodiments of dental auxiliary positioners and related devices described herein (e.g., the devices of FIGS. 1A, 1B, 25, 26, and 28A-28D).

FIG. 27A is a side view of an adjustable dental auxiliary positioner 2700a, in accordance with embodiments of the present technology. The positioner 2700a includes a plurality of registration elements 2702 configured to receive a patient's teeth, and one or more auxiliary supports 2704 each configured to position a corresponding auxiliary 2706 on a tooth. The registration elements 2702 and auxiliary supports 2704 can be identical or generally similar to the corresponding components of FIGS. 1A and 1B.

The registration elements 2702 are connected to one another by one or more couplings 2708. In some embodiments, the couplings 2708 allows for relative movement between adjacent registration elements 2702. Some or all of the couplings 2708 may be formed as a spring, which includes any suitable type of spring (e.g., coil, helical, conical, disc, leaf, or other suitable shape). When formed as a spring, the coupling 2708 can be configured to have spring properties depending on the treatment plan, and the spring may be a constant spring, variable spring, or a variable stiffness spring. The spring constant may also be designed into the spring to allow a desired motion of adjacent registration elements 2702 in response to an applied force. Some or all of the couplings 2708 may alternatively or additionally be formed of any suitable geometry, such as zig-zag, sinusoidal, S-shaped, circuitous, V-shaped, flat, and the like. Some or all of the couplings 2708 may also be formed as a suitable connection, such as a joint (e.g., ball and socket, heim joint, rose joint, etc.), flexible member, lattice structure, or other suitable coupling.

In some embodiments, the couplings 2708 allow articulation between the registration elements 2702 and may allow the distance between adjacent registration elements 2702 to be adjusted, e.g., to accommodate changes in tooth position as described herein. In some embodiments, the couplings 2708 allow relative movement between adjacent registration elements 2702 to allow each of the registration elements 2702 to be independently positioned on a respective tooth. In some embodiments, the couplings 2708 are designed and configured to allow a relative range of motion within a range from about 1 mm to about 5 mm and/or to exert a force within a range of from about 0.1 N to about 1 N.

The couplings 2708 can allow freedom in the relative distance and orientation between adjacent registration elements 2702. For example, in the course of a treatment plan, the patient's teeth may be scanned. By the time the positioner 2700a is applied to the patient's teeth (which can be several days or weeks after the initial scan), the teeth may have moved. The one or more couplings 2708 that allow relative motion between adjacent registration elements 2702 may reduce the possible need to fabricate a new positioner. The result can be greater efficiency, flexibility, and adjustability at the treatment location. The couplings 2708 may additionally provide advantages during manufacturing, handling, and/or installation. For example, the one or couplings 2708 may facilitate removal from an additive manufacturing platform and allow the positioner 2700a to absorb impacts and/or other forces associated with handling to a greater degree without failing.

FIG. 27B is a partially schematic side view of an adjustable dental auxiliary positioner 2700b, in accordance with embodiments of the present technology. In the illustrated embodiment, the coupling 2708 is depicted as a ball and socket joint that allows for articulated movement between adjacent registration elements 2702. For instance, a first registration element 2702 can include a ball 2710 extending therefrom, and a second registration element 2702 can include a socket 2712 configured to receive the ball 2710, and, optionally, a portion of a shaft 2714 connected to the ball 2710. In some embodiments, the socket 2712 elastically deforms in order to receive the ball 2710 therein, and then returns to its original shape to capture the ball 2710 within the socket 2712, for example. In some embodiments, the ball 2710 and socket 2712 are additively manufactured as a connected structure and no further assembly is required.

FIG. 27C is a partially schematic side view of an adjustable dental auxiliary positioner 2700c, in accordance with embodiments of the present technology. The positioner 2700c includes a base 2716 and a plurality of couplings 2718, such as spring couplings, for coupling each of the plurality of registration elements 2702 to the base 2716. The base 2716 may be a continuous structure that is shaped to correspond to a shape of a portion (e.g., a quadrant) of a patient's arch. In some embodiments, the base 2716 may be formed to be substantially parallel to the occlusal plane of the patient when the positioner 2700c is placed on the teeth of the patient.

In some embodiments, the couplings 2718 allow for relative movement between adjacent registration elements 2702 with respect to each other and the base 2716. Some or all of the couplings 2718 may be formed as a spring, which includes any suitable type of spring (e.g., coil, helical, conical, disc, leaf, or other suitable shape). When formed as a spring, the coupling 2718 can be configured to have spring properties depending on the treatment plan, and the spring may be a constant spring, variable spring, or a variable stiffness spring. The spring constant may also be designed into the spring to allow a desired motion of adjacent registration elements 2702 in response to an applied force. Some or all of the couplings 2718 may alternatively or additionally be formed of any suitable geometry, such as zig-zag, sinusoidal, S-shaped, circuitous, V-shaped, flat, and the like. Some or all of the couplings 2718 may also be formed as a suitable connection, flexible member, lattice structure, or other suitable coupling.

The couplings 2718 may be attached to the registration elements 2702 and the base 2716 through any suitable connection, such as an interference fit, bonding, mechanical fastening, integrally formed during manufacture, or any other suitable method for connecting the registration elements 2702 and the base 2716 with couplings 2718. In some embodiments, the couplings 2718 may be mounted within an aperture formed partially through the base 2716. In some embodiments, the couplings 2718 may be mounted to a base surface of the aperture. In some embodiments, the couplings 2718 may be mounted to an upper surface of the base 2716. The upper surface may be a surface that faces the occlusal surfaces of the teeth when the positioner 2700c is placed on the patient's teeth. The shape of the couplings 2718 and apertures can allow articulation of the registration elements 2702 along and/or about all three dimensions or axes with respect to the base 2716. For example, the shapes can allow for translation mesial-distally, buccal-lingually, and/or or occlusal-gingivally. The shapes may also provide for mesial-distal rotation, buccal-lingual rotation, and/or occlusal-gingival rotation.

In some embodiments, the couplings 2718 allow articulation between the registration elements 2702 and the base 2716, and may allow the distance between adjacent registration elements 2702 and the base 2716 to be adjusted, e.g., to accommodate changes in tooth position as described herein. In some embodiments, the couplings 2718 allow relative movement between registration elements 2702 and the base 2716 to allow each of the registration elements 2702 to be independently positioned on a respective tooth. In some embodiments, the couplings 2718 are designed and configured to allow a relative range of motion within a range from about 1 mm to about 5 mm, and/or to exert a force within a range of from about 0.1 N to about 1 N.

The couplings 2718 can allow freedom in the relative distance and orientation between adjacent registration elements 2702. For example, in the course of a treatment plan, the patient's teeth may be scanned. By the time the positioner 2700c is applied to the patient's teeth (which can be several days or weeks after the initial scan), the teeth may have moved. The one or more couplings 2718 that allow relative motion between adjacent registration elements 2702 may reduce the possible need to fabricate a new positioner. In some embodiments, a positioner 2700c with registration elements 2702 attached to a unitary base, such as base 2716, may provide for easier handling and manipulation by a clinician than a positioner where couplings directly couple the registration elements to each other. This may be because the unitary base 2716 provides a more rigid structure for the clinician to handle.

The couplings 2718 between the registration elements 2702 and the base 2716 can be tuned to have desired properties. For example, the couplings 2718 can be designed to have a desired strength, stiffness (e.g., spring constant “k”), restorative force, thickness, and the like. In some embodiments, the couplings 2718 are manufactured with the registration elements 2702 through an additive manufacturing process as described herein. Other coupling parameters that may be adjusted include, without limitation, spring diameter, diameter of a spring winding, number of windings, pitch, length, and the like. For example, the distance between the occlusal plane of the patient and an occlusal surface of each tooth may vary. In such embodiments, the length of the couplings 2718 may vary according to the distance between the occlusal plane and the occlusal surface of each tooth. For example, when a tooth is further from the occlusal plane, the length of the coupling 2718 that attaches a corresponding registration element 2702 for the tooth to the base 2716 may be longer than a coupling 2718 that connects a corresponding registration element 2702 for a tooth with an occlusal surface that is closer to the occlusal plane.

The positioner 2700c may optionally include one or more supports 2720 that connect the registration elements 2702 with the base 2716. In some embodiments, the supports 2720 may provide a rigid connection between the base 2716 and the registration elements 2702. In some embodiments, the supports 2720 may hold the registration elements 2702 in a position such that all of the registration elements 2702 are located with respect to their corresponding teeth according to a first tooth arrangement of the teeth. In some embodiments, if one or more of the teeth move away from the first tooth arrangement as described herein, then one or more of the supports 2720 may be fractured and/or removed to allow the registration element 2702 to be moved relative to the base 2716.

FIG. 27D is a partially schematic side view of an adjustable dental auxiliary positioner 2700d, in accordance with embodiments of the present technology. The positioner 2700d includes a base 2722 and a plurality of couplings 2724 for coupling each of the plurality of registration elements 2702 to the base 2722. The base 2722 may be a continuous structure that is shaped to correspond to a shape of a portion (e.g., a quadrant) of a patient's arch. In some embodiments, the base 2722 may be formed to be substantially parallel to the occlusal plane of the patient when the positioner 2700d is placed on the teeth of the patient.

In some embodiments, the couplings 2724 allow for relative movement between adjacent registration elements 2702 with respect to each other and the base 2722. The couplings 2724 may be formed as a type of captured coupling where an end of the registration element 2702 is captured by the base 2722 via an aperture 2726, as described in greater detail below. In some embodiments, the couplings 2724 allow articulation between the registration elements 2702 and the base 2722, and may allow the distance between adjacent registration elements 2702 and the base 2722 to be adjusted. In some embodiments, the couplings 2724 allow relative movement between registration elements 2702 and the base 2722 to allow each of the registration elements 2702 to be independently positioned on a respective tooth. In some embodiments, the couplings 2724 are designed and configured to allow a relative range of motion within a range from about 1 mm to about 5 mm, and/or rotation or angulation of +/−about 5 degrees, about 10 degrees, or about 15 degrees about each axis.

The couplings 2724 can accommodate changes in the relative distance and/or orientation between registration elements 2702 and the base 2722. For example, in the course of a treatment plan, the patient's teeth may be scanned. By the time the positioner 2700d is applied to the patient's teeth (which can be several days or weeks after the initial scan), the teeth may have moved. The one or more couplings 2724 that allow relative motion between adjacent registration elements 2702 and the base 2722 may reduce the possible need to fabricate a new positioner. The result can be greater efficiency, flexibility, and adjustability at the treatment location.

The couplings 2724 may include two parts: a base portion formed in the base 2722, and a captured portion including a shaft 2728 and head 2730. The base portion may include an aperture 2726 formed through the base 2722, and a socket 2732 formed in the base 2722 and extending from the aperture 2726. In some embodiments, the socket 2732 has a width in a buccal-lingual direction, and a height in an occlusal-gingival direction. The aperture 2726 may have a dimension, such as a diameter, which defines the size of the aperture 2726. The captured portion may include a shaft 2728 having a first end that extends from the registration element 2702, and a second end that extends from the head 2730. The shaft 2728 may have a length that extends from the registration element 2702 to the head 2730. The head 2730 may have a dimension (e.g., a diameter) that is larger than a corresponding dimension of the aperture 2726 in the base 2722 such that the head 2730 is captured by the aperture 2726. The base 2722 with the socket 2732 may be directly fabricated simultaneously with the captured portion (including the head 2730 and the shaft 2728) and the registration element 2702, such that the aperture 2726 captures the registration element 2702 and the captured portion as they are formed in a direct fabrication process.

The coupling 2724, including the base portion and the captured portion, can be tuned to have desired properties. For example, the shaft 2728 can be designed to have a desired length in order to provide adjustability for the distance between the base 2722 and a tooth. In addition, the aperture 2726 and shaft 2728 may be configured to provide for translation and/or angulation of the registration elements 2702 relative to the base 2722. For instance, the shape of the shaft 2728, head 2730, and socket 2732 can allow articulation of the registration elements 2702 along and/or about all three dimensions or axes with respect to the base 2722. For example, the shapes can allow for translation mesial-distally, buccal-lingually, and/or or occlusal-gingivally. The shapes may also provide for mesial-distal rotation, buccal-lingual rotation, and/or occlusal-gingival rotation.

The positioner 2700d may optionally include one or more supports (e.g., similar to the supports 2720 of FIG. 27C) that connect the registration elements 2702 with the base 2722. In some embodiments, the supports may provide a rigid connection between the base 2722 and the registration elements 2702. In some embodiments, the supports may hold the registration elements 2702 in a position such that all of the registration elements 2702 are located with respect to their corresponding teeth according to a first tooth arrangement of the teeth. In some embodiments, if one or more of the teeth move away from the first tooth arrangement as described herein, then one or more of the supports may be fractured and/or removed to allow the registration element 2702 to be moved relative to the base 2722.

Additional examples and features of adjustable dental auxiliary positioners that may be used with the present technology are provided in U.S. Patent Application Publication No. 2021/0259810, the disclosure of which is incorporated by reference herein in its entirety.

In some embodiments, the dental auxiliary positioners of the present disclosure include one or more registration elements having stabilizing features to enhance coupling of the registration elements to their corresponding teeth, which may improve the accuracy of auxiliary placement on the teeth. The stabilizing features can include extended surfaces at the buccal, lingual, and/or occlusal portions of the registration element to provide improved contact with the buccal, lingual, and/or occlusal surfaces of the tooth, respectively. Stabilizing features may be advantageous, for example, in embodiments where the dental auxiliary positioner includes a relatively small number of registration elements, such as no more than ten, nine, eight, seven, six, five, four, three, or two registration elements, or a single registration element only. Stabilizing features may also be beneficial in embodiments where the dental auxiliary positioner is separated into a plurality of smaller segments to place auxiliaries on a patient's teeth, e.g., to accommodate changes in tooth position as described above in connection with the method 2300 of FIG. 23. In such embodiments, each segment may include no more than ten, nine, eight, seven, six, five, four, three, or two registration elements, or a single registration element only.

FIGS. 28A-28D illustrate representative examples of registration elements with stabilizing features, in accordance with embodiments of the present technology. The embodiments of FIGS. 28A-28D can be used in any of the methods described herein, such as the method 2300 of FIG. 23. Moreover, any of the features of the embodiments of FIGS. 28A-28D can be combined with each other and/or with any of the other embodiments of dental auxiliary positioners and related devices described herein (e.g., the devices of FIGS. 1A, 1B, 25, 26, and 27A-27D).

FIGS. 28A and 28B are side and top views, respectively, of a registration element 2800a with stabilizing features, in accordance with embodiments of the present technology. The registration element 2800a includes a body 2802 configured to couple to a patient's tooth 2806. For instance, the body 2802 can include a first surface 2804 having a topography that is complementary to the occlusal surface of the tooth 2806, a second surface 2808 opposite the first surface 2804, and one or more sidewalls 2810 between the first surface 2804 and the second surface 2808. The registration element 2800a is coupled to an auxiliary support 2812 including an auxiliary for placement on the tooth 2806 (the auxiliary is obscured in FIGS. 28A and 28B). Optionally, the registration element 2800a can be coupled to a retention support 2814 that is configured to provide a biasing force to maintain close contact between the auxiliary and the tooth 2806.

In some embodiments, the registration element 2800a provides greater surface coverage of the tooth 2806. For instance, the first surface 2804 can cover at least 50%, 60%, 70%, 75%, 80%, 90%, 95%, 99%, or 100% of the occlusal surface of the tooth. The first surface 2804 can be extended in a buccal direction by a distance D₁ and/or in a lingual direction by a distance D₂ (e.g., relative to a default registration element as indicated by the broken lines) in order to provide greater occlusal coverage. The distance D₁ and the distance D₂ can each independently be at least 0.1 mm, 0.25 mm, 0.5 mm, 0.75 mm, 1 mm, 1.5 mm, or 2 mm. The distance D₁ can be less than the distance D₂, the distance D₁ can be greater than the distance D₂, or the distance D₁ can be equal to the distance D₂. Optionally, the second surface 2808 can also be extended in a buccal direction (e.g., by the distance D₁) and/or in a lingual direction (e.g., by the distance D₂).

Alternatively or in combination, some or all of the sidewalls 2810 of the registration element 2800a can be extended to provide greater surface coverage of the lingual, buccal, mesial, and/or distal surfaces of the tooth 2806. For instance, some or all of the sidewalls 2810 can be extended toward the tooth 2806 by a distance D₃ (e.g., relative to the default registration element as indicated by the broken lines). The distance D₃ can correspond to the height of the lowest cusp point of the tooth 2806. In some embodiments, the distance D₃ is at least 0.1 mm, 0.25 mm, 0.5 mm, 0.75 mm, 1 mm, 1.5 mm, or 2 mm.

FIG. 28C is a perspective view of a registration element 2800b with stabilizing features, in accordance with embodiments of the present technology. The registration element 2800b includes a body 2822 having an interior surface 2824 configured to couple to a tooth, and one or more sidewalls 2826 around the surface 2824. The registration element 2800b can be coupled to an auxiliary support 2828 for placing an auxiliary 2830 on the tooth. Optionally, the registration element 2800b can also be coupled to a retention support 2832 for biasing the auxiliary 2830 against the tooth.

The registration element 2800b can have a width W (e.g., measured along a buccal-lingual direction) that provides greater coverage of the occlusal surface of the tooth. For instance, the surface 2824 of the registration element 2800b can cover at least 50%, 60%, 70%, 75%, 80%, 90%, 95%, 99%, or 100% of the occlusal surface of the tooth. Alternatively or in combination, the registration element 2800b can have a height H (e.g., measured along a gingival-occlusal direction) that provides greater coverage of the buccal, lingual, mesial, and/or distal surfaces of the tooth. For instance, the sidewalls 2826 of the registration element 2800b can cover at least 10%, 20%, 25%, 30%, 40%, 50%, 60%, 70%, 75%, 80%, 90%, 95%, 99%, or 100% of the buccal, lingual, mesial, and/or distal surfaces of the tooth. In the illustrated embodiment, for example, the buccal sidewall 2826 of the registration element 2800b has been extended to provide greater coverage of the buccal surface of the tooth. Accordingly, the auxiliary support 2828 can be partially or fully embedded within the buccal sidewall 2826, rather than being positioned above the sidewall 2826. In such embodiments, certain components of the auxiliary support 2828 can be truncated or omitted altogether, such as the frame and/or bridge.

FIG. 28D is a perspective view of a dental auxiliary positioner 2800c including at least one registration element with stabilizing features, in accordance with embodiments of the present technology. The positioner 2800c includes a plurality of registration elements 2840a-2840g (collectively, “registration elements 2840”) configured to receive a patient's teeth. Although FIG. 28D illustrates eight registration elements 2840, in other embodiments, the positioner 2800c can include a different number of registration elements 2840, such as two, three, four, five, six, seven, or more registration elements 2840. Any of the registration elements 2840 can include an auxiliary support having an auxiliary for placement on a tooth and/or a retention support. Optionally, any of the registration elements 2840 may not include an auxiliary support and/or a retention support.

Some or all of the registration elements 2840 can include stabilizing features to enhance coupling to the patient's teeth, as described herein. In the illustrated embodiment, for example, the registration element 2840b that is the penultimate registration element in the positioner 2800c (e.g., adjacent to the distalmost registration element 2840a) has stabilizing features (e.g., extended occlusal coverage). The remaining registration elements 2840a, 2840c-2840g can have a default geometry without stabilizing features, such that the registration element 2840b is the only registration element 2840 with stabilizing features. Alternatively, some or all of the remaining registration elements 2840a, 2840c-2840g can also have stabilizing features. Moreover, any of the registration elements 2840 illustrated in FIG. 28D can be provided with or without stabilizing features, as desired.

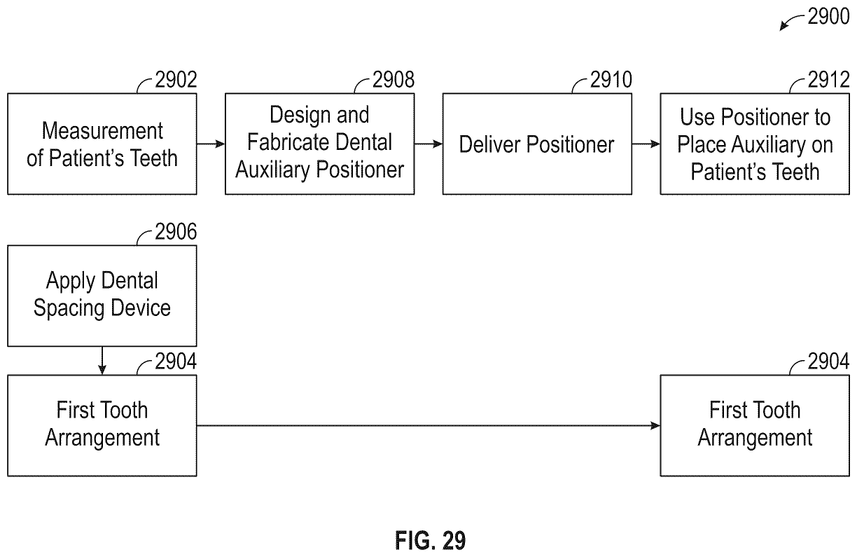

In some embodiments, the present technology provides methods for placing auxiliaries on teeth that are prone to movement (e.g., due to proximity to missing, unerupted, or actively erupting teeth) by applying a dental spacing device to inhibit tooth movement until the dental auxiliary positioner is ready for use, e.g., as described in connection with FIGS. 29-34B below. As described herein, the dental auxiliary positioner may be designed based on measurements of the patient's teeth (e.g., scan data) taken at an initial time, but there may be a significant delay (e.g., four to six weeks) between the initial time at which the measurements were taken, and the subsequent time at which the positioner is actually used to place the auxiliary. Teeth that are proximate to a space left by a missing, unerupted, or erupting tooth may move relative to the space during the delay period, such that the positioner may no longer fit properly on the patient's teeth and/or may not locate the auxiliary at the proper position on the tooth. To address these and other challenges, a dental spacing device can be placed into and/or proximate to the space to inhibit tooth movement relative to the space (e.g., prevent the tooth from moving toward and/or away from the space), thereby maintaining the tooth in its initial position to ensure the positioner will fit properly on the tooth and/or locate the auxiliary correctly on the tooth, even if significant time has elapsed since the initial measurement. This approach can improve predictability of treatment, avoid treatment delays due to redesign of positioners that no longer fit properly, and/or provide improved treatment outcomes.

FIG. 29 is a schematic block diagram illustrating a workflow 2900 for placing a dental auxiliary on a tooth, in accordance with embodiments of the present technology. The workflow 2900 can be used to accurately place a dental auxiliary on the tooth, even if the tooth is susceptible to movement (e.g., due to proximity to a missing, unerupted, or erupting tooth).

The workflow 2900 includes obtaining a measurement of the patient's teeth (block 2902). The measurement can be based on any data type that provides information on the positions and orientations of some or all of the teeth, such as photographs and/or videos (as captured on, e.g., a mobile computing device such as a smartphone, or another suitable device with a camera), scan data (e.g., intraoral and/or extraoral scans), MRI data, and/or radiographic data (e.g., standard x-ray data such as bitewing x-ray data, panoramic x-ray data, cephalometric x-ray data, CT data, CBCT data, fluoroscopy data). In some embodiments, for example, the measurement is or includes scan data obtained using an intraoral scanner, as described herein.

The measurement can be taken while the patient's teeth are in a first tooth arrangement (block 2904). In some embodiments, the first tooth arrangement is a pre-treatment tooth arrangement of the patient's teeth, such as a tooth arrangement before any dental appliances have been applied to the patient's teeth in accordance with a treatment plan. Alternatively, the first tooth arrangement can be an intermediate tooth arrangement of the patient's teeth, such as a tooth arrangement during an intermediate stage of a treatment plan in which one or more dental appliances have already been applied to the patient's teeth. The first measurement can be obtained for all of the patient's teeth, or can be obtained for only a subset of the patient's teeth (e.g., only the teeth that will receive auxiliaries, and/or only the teeth that are likely to move).

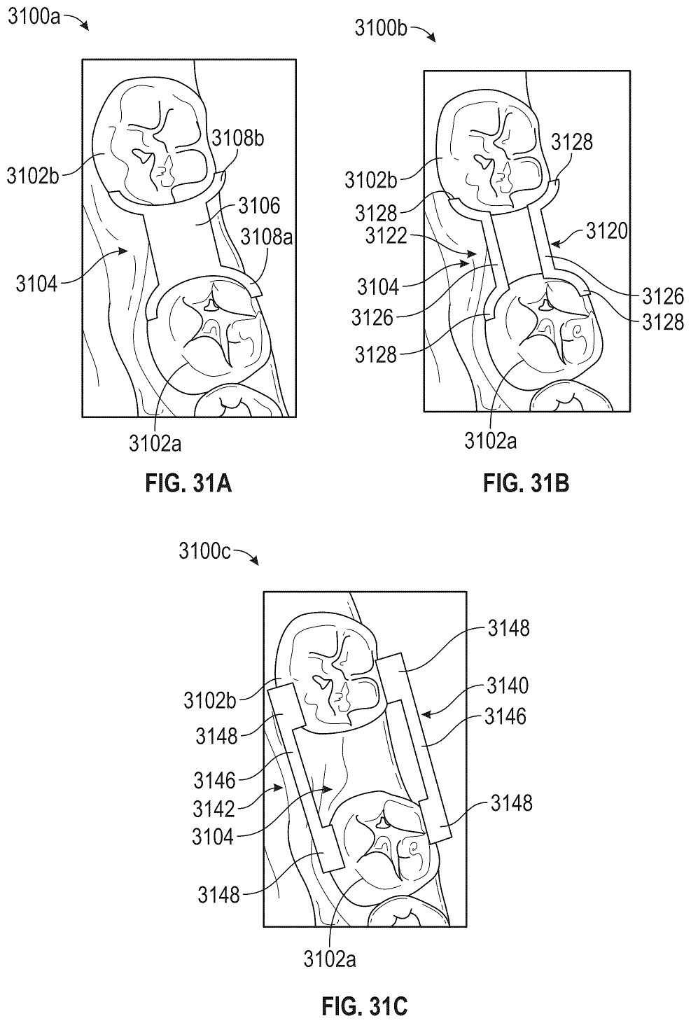

The workflow 2900 also includes applying a dental spacing device to the patient's teeth (block 2906). The dental spacing device can be applied to the patient's teeth at any suitable time, such as before, concurrently with, or after obtaining the measurement of the patient's teeth. For instance, the dental spacing device can be placed shortly after the measurement (e.g., on the same day, or within one day, two days, or three days). The dental spacing device can be configured to maintain the positions of some or all of the patient's teeth, e.g., in the same or a similar position as in the first tooth arrangement. For example, the dental spacing device can inhibit movement of a tooth that is proximate to a missing, unerupted, or erupting tooth, e.g., via a spacer portion that is configured to fit into or proximate to the space proximate to the tooth to prevent the tooth from moving relative to the space. Additional details and examples of dental spacing devices are described below, e.g., in connection with FIGS. 31A-34B.

The first measurement of the patient's teeth in the first tooth arrangement can be used to design and fabricate a dental auxiliary positioner (block 2908). The positioner can be configured to place one or more auxiliaries (e.g., attachments, buttons, power arms, brackets, wires, splints, distalizers) on one or more of the patient's teeth. The positioner can include any of the features of the embodiments of FIGS. 1A and 1B. For example, the positioner can include one or more registration elements configured to receive one or more teeth of the patient, and at least one auxiliary support coupled to a registration element and to an auxiliary in order to position an auxiliary at a predetermined location on a tooth. In some embodiments, the positioner is adjustable (e.g., as described in connection with FIGS. 26-28D), while in other embodiments, the positioner is not adjustable. In some embodiments, the positioner is designed based on the measurement of the patient's teeth, such that the positioner is customized to the first tooth arrangement of the patient's teeth. For instance, the geometry and locations of the registration elements and/or auxiliary supports can correspond to the geometry and locations of the teeth in the first tooth arrangement. The positioner can be fabricated using any suitable technique, such as a direct fabrication technique using one or more additive manufacturing processes, e.g., as described above in connection with block 2206 of FIG. 22.

After fabrication, the positioner can be delivered to the clinician or patient (block 2910). The positioner can then be used to place an auxiliary on the patient's teeth (block 2912). For example, the positioner can be placed on the patient's teeth to position the auxiliary at a predetermined location on a surface of a tooth, the auxiliary can be mounted to the tooth via an adhesive (e.g., a UV-curable adhesive), and the positioner can then be separated from the auxiliary and removed from the patient's teeth while the auxiliary remains in place on the tooth.

The dental spacing device can remain in place during some or all of the time period between the measurement of the patient's teeth (block 2902) and the placement of the auxiliary on the teeth (block 2912). For instance, the time period can be at least one week, two weeks, three weeks, four weeks, five weeks, six weeks, or more. In some embodiments, the dental spacing device remains continuously on the teeth for the entirety of the time period (e.g., if the device is affixed to the teeth via bonding), while in other embodiments, the dental spacing device may be temporarily removed from the teeth for brief periods (e.g., when the patient is eating, brushing their teeth, etc.). The dental spacing device can be removed shortly before the positioner is used to place the auxiliary, such as on the same day (e.g., at the same appointment) or no more than three days, two days, or one day before auxiliary placement.

The presence of the dental auxiliary positioner can inhibit movement of at least one tooth, such that at the time the positioner is ready for use, the tooth is still in the same or substantially the same position as in the first tooth arrangement, and the positioner can fit properly to place the auxiliary at the appropriate location on the tooth. For instance, the maximum translation distance of the tooth can be no more than 2 mm, 1 mm, 0.5 mm, 0.2 mm, or 0.1 mm; and/or the maximum rotation of the tooth can be no more than 1 degree, 2 degrees, 3 degrees, 4 degrees, 5 degrees, 6 degrees, 7 degrees, 8 degrees, 9 degrees, or 10 degrees. In some embodiments, all of the patient's teeth are in the same or substantially the same positions at the time the positioner is ready for use. Alternatively, it may be permissible for teeth that are not intended to receive auxiliaries and/or not necessary for proper fitting of the positioner to move, as long as teeth that are intended to receive auxiliaries and/or are necessary for proper fitting of the positioner remain in the same or substantially the same positions as in the first tooth arrangement at the time the positioner is ready for use.

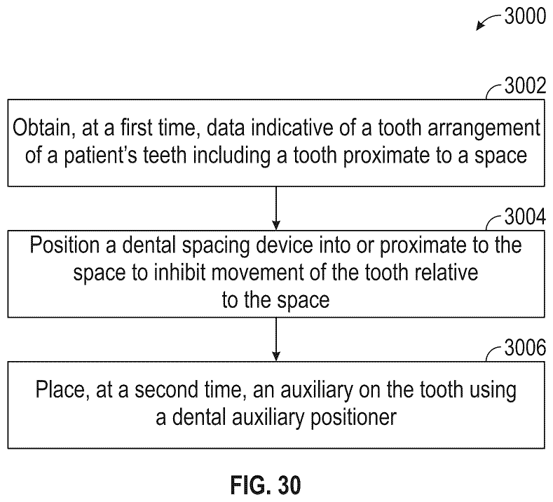

FIG. 30 is a flow diagram illustrating a method 3000 for placing a dental auxiliary on a tooth, in accordance with embodiments of the present technology. For instance, the method 3000 can be used to place an auxiliary on a tooth that is prone to movement, such as a tooth that is proximate to a space associated with a missing, unerupted, or erupting tooth. The method 3000 can be combined with and/or performed as part of the workflow 2900 of FIG. 29. Some or all of the processes of the method 3000 can be performed manually (e.g., by a clinician or other healthcare provider), and/or some or all of the processes of the method 3000 can be implemented as computer-readable instructions (e.g., program code) that are configured to be executed by one or more processors of a computing device.

The method 3000 can begin at block 3002 with obtaining, at a first time, data indicative of a tooth arrangement of a patient's teeth, including a tooth proximate to a space (e.g., a space associated with a missing, unerupted, or erupting tooth). The data can be any data type that provides information on the positions and orientations of the patient's teeth at the first time, such as photographs, videos, scan data, MRI data, and/or radiographic data, etc. The data can be obtained for all of the patient's teeth, or can be obtained for only a subset of the patient's teeth (e.g., only the teeth that will receive auxiliaries). The data can be used to design a dental auxiliary positioner for placing one or more auxiliaries on the teeth, as described further below with respect to block 3006.

At block 3004, the method 3000 can include positioning a dental spacing device into or proximate to the space proximate to the tooth. The dental spacing device can be configured to inhibit movement of the tooth relative to the space. For instance, the dental spacing device can include a spacer portion (e.g., a block, wire, or other solid component) that fills some or all of the space to block tooth movement toward the space. Alternatively or in combination, the spacer portion can be positioned near the space and can be affixed to another tooth that serves as a mounting element to prevent tooth movement toward and/or away from the space. The dental spacing device can be coupled to one or more teeth to secure the spacer portion within the patient's mouth. The coupling can be a fixed coupling that is not removable by the patient, such as a coupling using curable adhesives (e.g., UV-curable adhesives) and/or a secure mechanical fit (e.g., bands and/or other components that fit tightly around one or more teeth). Alternatively, the coupling can be removable by the patient, such a coupling using a mechanical fit that is sufficiently loose to permit manual removal without the aid of specialized tools. Additional details and examples of dental spacing devices that may be used are described, e.g., in connection with FIGS. 31A-34B below.

In some embodiments, the dental spacing device is a generic device that can be adjusted (e.g., resized) for the particular tooth arrangement of the patient. For instance, the size of the spacer portion can be adjusted (e.g., lengthened and/or expanded, or shortened and/or compressed) to conform to the size of the space. Adjustability of the spacer portion can be achieved via a mechanical adjustment mechanism (e.g., a telescoping mechanism), flexible properties of the spacer portion (e.g., the spacer portion can be made out of a deformable material and/or can have a deformable structure), or suitable combinations thereof. Optionally, a plurality of dental spacing devices with different sizes can be provided (e.g., as a kit), and the clinician can select the device having the size that is closest to the size of the space for use. In other embodiments, however, the dental spacing device can be manufactured in a configuration that is customized to the particular patient, e.g., via chairside 3D printing.

At block 3006, the method 3000 can continue with placing, at a second time after the first time, an auxiliary on the tooth using a dental auxiliary positioner. The dental auxiliary positioner can have a configuration based on the data of the arrangement of the patient's teeth at the first time. For instance, the dental auxiliary positioner can be designed to fit the arrangement of the patient's teeth at the first time, such that the locations and geometry of the registration element(s) and auxiliary support(s) of the positioner correspond to the locations and geometry of the patient's teeth in the arrangement at the first time.

The second time can be at least one week, two weeks, three weeks, four weeks, five weeks, six weeks, or more after the first time. In some embodiments, the dental spacing device is worn on the patient for at least 50%, 60%, 70%, 75%, 80%, 90%, 95%, 99%, or 100% of the time elapsed between the first time and the second time. The dental spacing device may be worn continuously, or may be worn substantially continuously with only brief periods of removal for brushing, eating, etc. Accordingly, the dental spacing device can inhibit movement of the tooth relative to the space (e.g., toward and/or away from the space), thus ensuring that the tooth is still in the same or substantially the same position at the second time so that the dental auxiliary positioner fits properly on the tooth.

The process of block 3006 can include removing the dental spacing device from the patient's teeth before applying the dental auxiliary positioner to the teeth to place the auxiliary. For instance, the dental spacing device can be removed immediately before auxiliary placement (e.g., at the same appointment and/or on the same day), or a short time period before auxiliary placement (e.g., within one day, two days, or three days beforehand). As described elsewhere herein, the auxiliary placement process can include mounting the auxiliary to the tooth using an adhesive, separating the auxiliary from the dental auxiliary positioner, then removing the dental auxiliary positioner from the patient's teeth while leaving the auxiliary bonded in place on the tooth.

The method 3000 illustrated in FIG. 30 can be modified in many different ways. For example, some of the processes of the method 3000 can be omitted, such as the process of block 3002. The method 3000 can include additional processes not shown in FIG. 30, and/or the order of the processes shown in FIG. 30 can be varied as desired. Moreover, although the above processes of the method 3000 are described with respect to a single auxiliary on a single tooth, the method 3000 can be used to inhibit movements of multiple teeth and/or to place multiple auxiliaries on the teeth.

FIGS. 31A-34B illustrate representative examples of dental spacing devices for inhibiting tooth movement, in accordance with embodiments of the present technology. The embodiments of FIGS. 31A-34B can be used in any of the methods described herein, such as the method 3000 of FIG. 30. Moreover, any of the features of the embodiments of FIGS. 31A-34B can be combined with each other.

FIG. 31A is a partially schematic top view of a dental spacing device 3100a, in accordance with embodiments of the present technology. The device 3100a can be used to inhibit movement of at least one tooth 3102a relative to a space 3104 in a patient's dentition (e.g., a space left by a missing, unerupted, or erupting tooth). The device 3100a includes a spacer portion 3106 configured to fit into the space 3104, a first fixation portion 3108a configured to couple to the tooth 3102a, and a second fixation portion 3108b configured to couple to another tooth 3102b proximate to the space 3104 (e.g., at an opposite side of the space 3104 as the tooth 3102a).

The spacer portion 3106 can physically obstruct the tooth 3102a and/or the tooth 3102b from tipping, inclining, translating, or otherwise moving toward the space 3104. In the illustrated embodiment, the spacer portion 3106 is a block that fills a majority of or the entirety of the space 3104. Alternatively, the spacer portion 3106 can be a wire, crossbar, rod, or other thin elongate member that spans the space 3104 but fills less than all of the space 3104. If there is an actively erupting tooth in the space 3104, the spacer portion 3106 can be configured to accommodate the erupting tooth by not occluding the eruption path (e.g., the spacer portion 3106 can include a sufficient clearance gap to allow for safe eruption of the tooth). The spacer portion 3106 can have a length that is the same as or similar to the length of the space 3104. In some embodiments, the length of the spacer portion 3106 is at least 80%, 90%, 95%, 99%, or 100% of the length of the space 3104. The spacer portion 3106 can be made partially or entirely out of a rigid material (e.g., a high modulus polymer or a metal) that is sufficiently stiff to resist deformation and/or deflection by applied forces from the teeth 3102a, 3102b. Alternatively or in combination, the dimensions of the spacer portion 3106 (e.g., width, thickness, and/or diameter) can be sufficiently large to impart rigidity to the spacer portion 3106.

The fixation portions 3108a, 3108b (collectively, “fixation portions 3108”) can be connected to the spacer portion 3106 to mount the spacer portion 3106 to the respective teeth 3102a, 3102b (collectively, “teeth 3102”). Optionally, the coupling between the fixation portions 3108 and the teeth 3102 can also prevent the teeth 3102 from tipping, inclining, translating, or otherwise moving away from the space 3104. In the illustrated embodiment, the first fixation portion 3108a is connected to a first side of the spacer portion 3106 and is coupled to the tooth 3102a, and the second fixation portion 3108b is connected to a second, opposite side of the spacer portion 3106 and is coupled to the other tooth 3102b. The fixation portions 3108 can be coupled to the teeth 3102 using any suitable technique, such as adhesives (e.g., UV curable adhesives), mechanical fit, or suitable combinations thereof.

Each fixation portion 3108 can include a surface that engages a corresponding surface of the coupled tooth 3102. Although the fixation portions 3108 are depicted as engaging the buccal, lingual, and interproximal surfaces of each tooth 3102, in other embodiments, the fixation portions 3108 can engage the buccal surface only, the lingual surface only, the interproximal surface only, the buccal and interproximal surface only, or the lingual and interproximal surfaces only. Moreover, although the fixation portions 3108 are depicted as extending along only a portion of the circumference of each tooth 3102, in other embodiments, the fixation portions 3108 can extend around the entire tooth circumference (e.g., similar to a molar band).

Optionally, the second fixation portion 3108b can be omitted, such that the device 3100a is coupled only to the tooth 3102a via the first fixation portion 3108a. In such embodiments, the second side of the spacer portion 3106 can abut a surface of the tooth 3102b (e.g., the buccal, lingual, and/or interproximal surface), e.g., without being coupled to the tooth 3102b via adhesives and/or mechanical fixation. This configuration can be used to inhibit movement of the teeth 3102 toward the space 3104, but may not inhibit movement of the teeth 3102 away from the space 3104.

FIG. 31B is a partially schematic top view of a dental spacing device 3100b, in accordance with embodiments of the present technology. The device 3100b can be used to inhibit movement of at least one tooth 3102a relative to a space 3104 in a patient's dentition (e.g., a space left by a missing, unerupted, or erupting tooth). In the illustrated embodiment, the device 3100b includes a lingual element 3120 in a lingual side of the space 3104, and a buccal element 3122 in a buccal side of the space 3104. The lingual element 3120 and buccal element 3122 can each include a spacer portion 3126 and one or more fixation portions 3128 connected to the spacer portion 3126, which may be generally similar to the corresponding components of FIG. 31A.

In the illustrated embodiment, the spacer portion 3126 of the lingual element 3120 is positioned within and spans the lingual side of the space 3104, and the spacer portion 3126 of the buccal element 3122 is positioned within and spans the buccal side of the space 3104. The spacer portions 3126 can resist movements of the teeth 3102 toward and/or away from the space 3104. Although the spacer portions 3126 are depicted as being wires, in other embodiments, one or both of the spacer portions 3126 instead be configured as a block (e.g., similar to the embodiment shown in FIG. 31A). If there is an actively erupting tooth in the space 3104, the spacer portions 3126 can be configured to accommodate the erupting tooth by not occluding the eruption path (e.g., the spacer portions 3126 can include a sufficient clearance gap to allow for safe eruption of the tooth).

In the illustrated embodiment, the fixation portions 3128 of the lingual element 3120 are coupled to the lingual and/or interproximal surfaces of the teeth 3102, and the fixation portions 3128 of the buccal element 3122 are coupled to the buccal and/or interproximal surfaces of the teeth 3102. Optionally, the fixation portions 3128 for the tooth 3102a can be combined into a single unitary component (e.g., a single band that extends partially or entirely around the tooth 3102a), and/or the fixation portions 3128 for the tooth 3102b can be combined into a single unitary component (e.g., a single band that extends partially or entirely around the tooth 3102b), such that the lingual element 3120 and the buccal element 3122 are connected to each other via the fixation portions 3128. Moreover, although FIG. 31B illustrates a device 3100b with both a lingual element 3120 and a buccal element 3122, in other embodiments, the device 3100b can include the lingual element 3120 only or the buccal element 3122 only.

FIG. 31C is a partially schematic top view of a dental spacing device 3100c configured in accordance with embodiments of the present technology. The device 3100c can be used to inhibit movement of at least one tooth 3102a relative to a space 3104 in a patient's dentition (e.g., a space left by a missing, unerupted, or erupting tooth). In the illustrated embodiment, the device 3100c includes a lingual element 3140 proximate to a lingual side of the space 3104, and a buccal element 3142 proximate to a buccal side of the space 3104. The lingual element 3140 and buccal element 3142 can each include a spacer portion 3146 and one or more fixation portions 3148 coupled to the spacer portion 3146, which may be generally similar to the corresponding components of FIGS. 31A and 31B. The lingual element 3140 and buccal element 3142 can differ from the lingual element 3120 and buccal element 3122 of FIG. 31B, in that the lingual element 3140 and buccal element 3142 are positioned entirely outside of the space 3104.

In the illustrated embodiment, the spacer portion 3146 of the lingual element 3140 is positioned lingual to the lingual side of the space 3104, and the fixation portions 3148 of the lingual element 3140 are coupled to the lingual surfaces of the teeth 3102. The spacer portion 3146 of the buccal element 3142 is positioned buccal to the buccal side of the space 3104, and the fixation portions 3148 of the buccal element 3142 are coupled to the buccal surfaces of the teeth 3102. The spacer portions 3146 can resist movements of the teeth 3102 toward and/or away from the space 3104. Although the spacer portions 3146 are depicted as being wires, in other embodiments, one or both of the spacer portions 3146 instead be configured as a block (e.g., similar to the embodiment shown in FIG. 31A). Moreover, although FIG. 31C illustrates a device 3100c with both a lingual element 3140 and a buccal element 3142, in other embodiments, the device 3100c can include the lingual element 3140 only or the buccal element 3142 only.

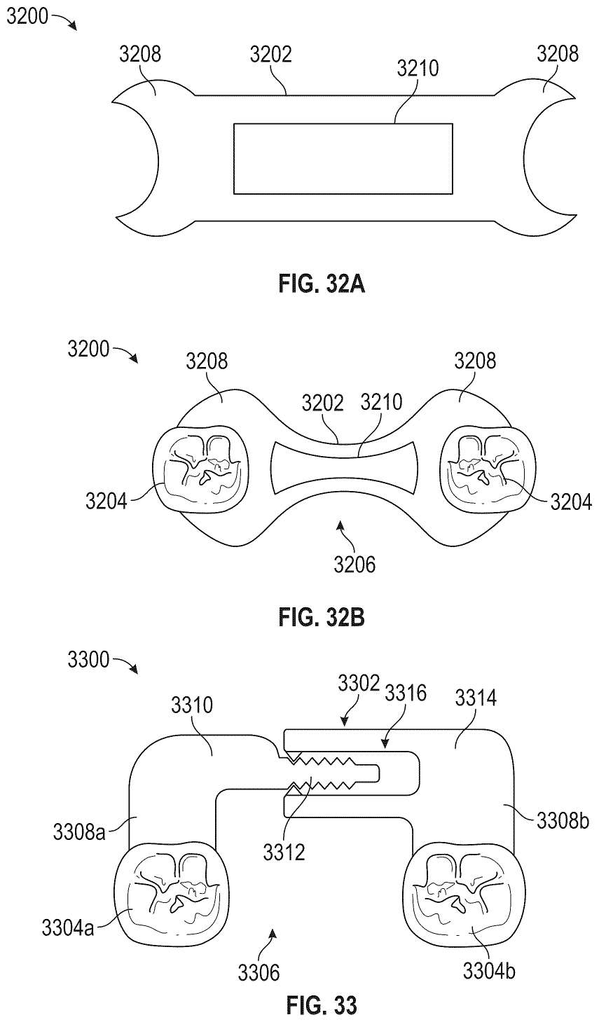

FIGS. 32A and 32B are partially schematic top views of a dental spacing device 3200 with a deformable spacer portion 3202, in accordance with embodiments of the present technology. The device 3200 can be used to inhibit movement of at least one tooth 3204 relative to a space 3206 in a patient's dentition (e.g., a space left by a missing, unerupted, or erupting tooth). The device 3200 includes the spacer portion 3202, and at least one fixation portion 3208 connected to the spacer portion 3202 and configured to couple to at least one corresponding tooth 3204.

The spacer portion 3202 can be configured to deform in order to fit into the space 3206. For example, the spacer portion 3202 can be composed partially or entirely out of a deformable material (e.g., a low modulus polymer) that is deformable (e.g., compressible and/or expandable). Alternatively or in combination, the spacer portion 3202 can have a deformable structure. In the illustrated embodiment, for example, the spacer portion 3202 includes a cavity 3210 (e.g., a slot, hole, groove, recess) formed therein to reduce the overall stiffness of the spacer portion 3202. Other types of deformable structures that may be used for the spacer portion 3202 include mesh and/or porous structures, springs, pleats, compliant mechanisms, and/or suitable combinations thereof.

As shown in FIG. 32A, when the device 3200 is in an unloaded configuration (e.g., before placement on the patient's teeth), the spacer portion 3202 can be in an undeformed state with an initial resting length. As shown in FIG. 32B, when the device 3200 is placed into a space 3206 having a length that is shorter than the resting length of the spacer portion 3202, the spacer portion 3202 can be compressed, collapsed, or otherwise deformed to a shorter length in order to fit into the space 3206. This approach can advantageously allow the device 3200 to fit into differently-sized spaces between teeth. The spacer portion 3202 can still have sufficient stiffness to resist movement of the teeth 3204 relative to the space 3206. In some embodiments, the force exerted on the teeth 3204 by the spacer portion 3202 attempting to return to its resting length may prevent the teeth 3204 from moving toward the space 3206.

FIG. 33 is a partially schematic top view of a dental spacing device 3300 with an adjustable spacer portion 3302, in accordance with embodiments of the present technology. The device 3300 can be used to inhibit movement of one or more teeth 3304a, 3304b (collectively, “teeth 3304”) relative to a space 3306 in a patient's dentition (e.g., a space left by a missing, unerupted, or erupting tooth). The device 3300 includes the spacer portion 3302, a first fixation portion 3308a configured to couple to a first tooth 3304a, and a second fixation portion 3308b configured to couple to a second tooth 3304b.

The spacer portion 3302 includes a mechanical adjustment mechanism that allows the length of the spacer portion 3302 to be modified (e.g., lengthened and/or shortened) to accommodate differently-sized spaces between teeth. In the illustrated embodiment, for example, the spacer portion 3302 includes a first section 3310 connected to the first fixation portion 3308a and including a projection 3312, and a second section 3314 connected to the second fixation portion 3308b and including a lumen 3316 formed therein. The projection 3312 can be slidably received within the lumen 3316, thereby forming a telescopic assembly that can be adjusted to increase or decrease the overall length of the spacer portion 3302. The exterior surfaces of the projection 3312 and interior surfaces of the lumen 3316 can include locking features (e.g., mating ratchet teeth) to maintain the projection 3312 at a specific position within the lumen 3316, thereby fixing the length of the spacer portion 3302 to inhibit movement of the teeth 3304.

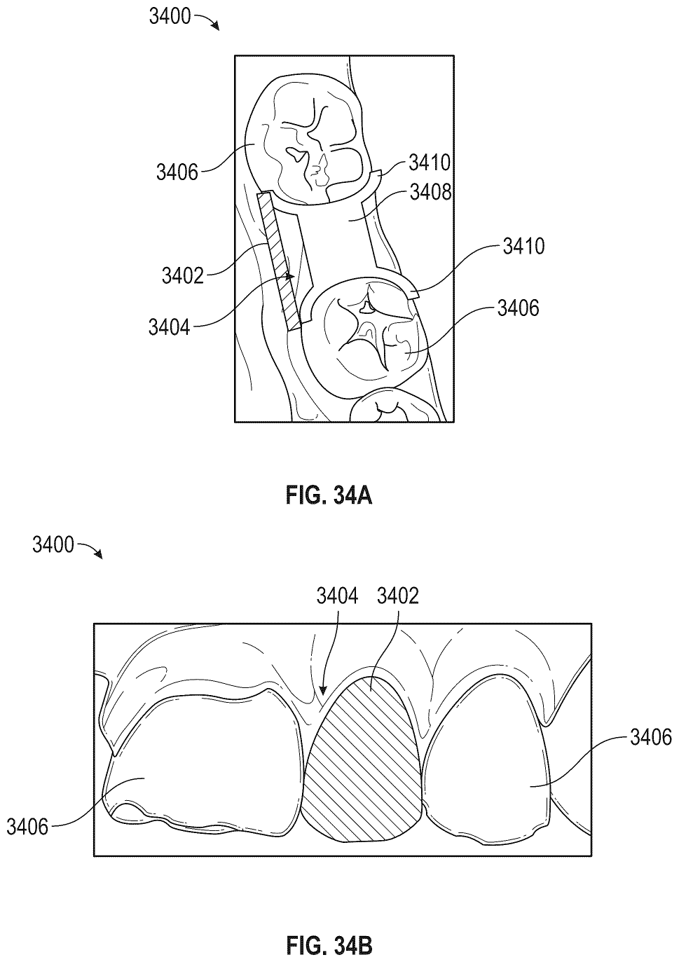

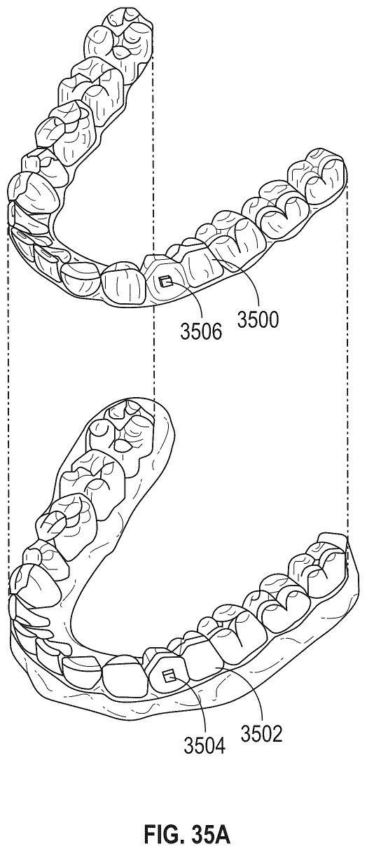

FIGS. 34A and 34B are partially schematic top and side views, respectively, of a dental spacing device 3400 with an aesthetic portion 3402, in accordance with embodiments of the present technology. The aesthetic portion 3402 can be a cover, plate, pontic, veneer, or other component that is configured to partially or entirely obscure a space 3404 in the patient's dentition due to a missing, unerupted, or erupting tooth. For instance, the aesthetic portion 3402 can include a visual representation of one or more teeth to disguise the presence of the space 3404. The aesthetic portion 3402 can be positioned at or proximate to the buccal side of the space 3404 such when the patient's teeth 3406 are exposed (e.g., due to the patient smiling), the space 3404 is not visible. The device 3400 can further include a spacer portion 3408 configured to inhibit movement of one or more teeth 3406, and at least one fixation portion 3410 connected to the spacer portion 3408 and configured to couple to at least one corresponding tooth 3406. Accordingly, the device 3400 can be used to concurrently inhibit tooth movement while also covering the space 3404 to improve the appearance of the patient's smile. Although the spacer portion 3408 and fixation portions 3410 are depicted as being similar to the embodiment of FIG. 31A, the spacer portion 3408 and fixation portions 3410 can alternatively be configured according to any of the other embodiments described herein.

In the illustrated embodiment, the aesthetic portion 3402 is a discrete component that is separate from the spacer portion 3408 and the fixation portions 3410. In such embodiments, the aesthetic portion 3402 can be coupled to the spacer portion 3408, to one or both fixation portions 3410, or a combination thereof using any suitable attachment technique, such as adhesives, mechanical fit, fasteners, etc. The aesthetic portion 3402 can be positioned buccal to and coupled to the buccal side of the spacer portion 3408 and/or the fixation portions 3410. In other embodiments, however, the aesthetic portion 3402 can be part of the spacer portion 3408, rather than being a separate discrete component. In such embodiments, the aesthetic portion 3402 can be a visual representation of one or more teeth that is formed directly on the buccal surface of the spacer portion 3408 (e.g., as an image, engraving, coating, molding, etc.).

### IV. Dental Appliances and Associated Methods

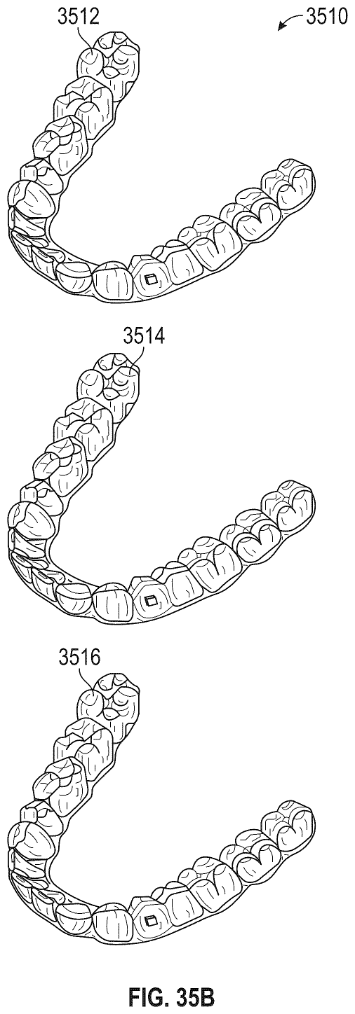

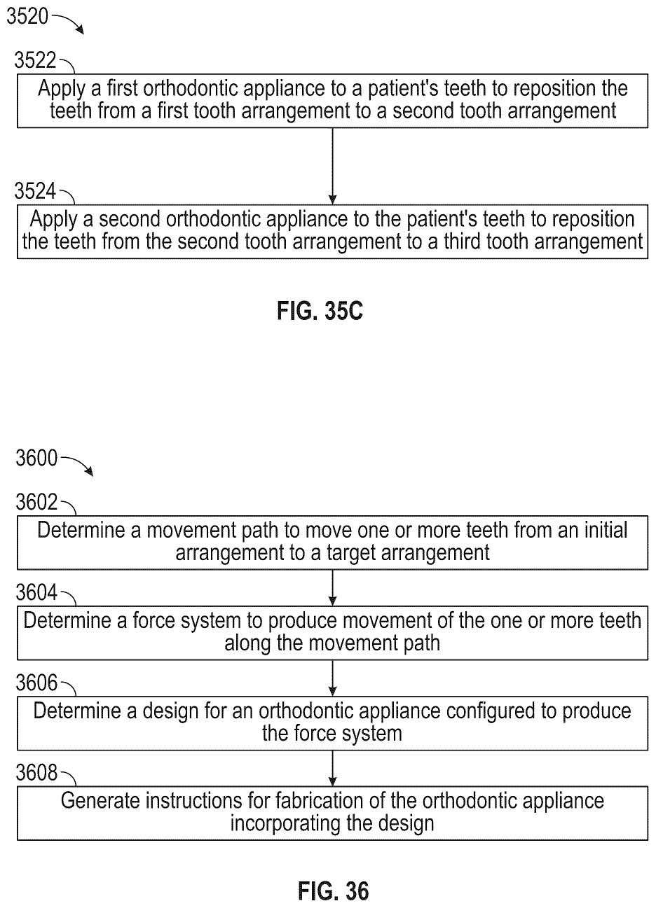

FIG. 35A illustrates a representative example of a tooth repositioning appliance 3500 configured in accordance with embodiments of the present technology. The appliance 3500 can be used in combination with any of the systems, methods, and devices described herein. The appliance 3500 (also referred to herein as an “aligner”) can be worn by a patient in order to achieve an incremental repositioning of individual teeth 3502 in the jaw. The appliance 3500 can include a shell (e.g., a continuous polymeric shell or a segmented shell) having teeth-receiving cavities that receive and resiliently reposition the teeth. The appliance 3500 or portion(s) thereof may be indirectly fabricated using a physical model of teeth. For example, an appliance (e.g., polymeric appliance) can be formed using a physical model of teeth and a sheet of suitable layers of polymeric material. In some embodiments, a physical appliance is directly fabricated, e.g., using additive manufacturing techniques, from a digital model of an appliance.

The appliance 3500 can fit over all teeth present in an upper or lower jaw, or less than all of the teeth. The appliance 3500 can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance 3500 can be a generic appliance configured to receive the teeth, but not necessarily shaped to match the topography of the patient's teeth. In some cases, only certain teeth received by the appliance 3500 are repositioned by the appliance 3500 while other teeth can provide a base or mounting region for holding the appliance 3500 in place as it applies force against the tooth or teeth targeted for repositioning. In some cases, some, most, or even all of the teeth can be repositioned at some point during treatment. Teeth that are moved can also serve as a base or mounting region for holding the appliance as it is worn by the patient. In preferred embodiments, no wires or other means are provided for holding the appliance 3500 in place over the teeth. In some cases, however, it may be desirable or necessary to provide individual attachments 3504 or other auxiliaries (e.g., buttons) on teeth 3502 with corresponding receptacles 3506 or apertures in the appliance 3500 so that the appliance 3500 can apply a selected force on the tooth. Representative examples of appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the url “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 35B illustrates a tooth repositioning system 3510 including a plurality of appliances 3512, 3514, 3516, in accordance with embodiments of the present technology. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 3510 can include a first appliance 3512 corresponding to an initial tooth arrangement, one or more intermediate appliances 3514 corresponding to one or more intermediate arrangements, and a final appliance 3516 corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

FIG. 35C illustrates a method 3520 of orthodontic treatment using a plurality of appliances, in accordance with embodiments of the present technology. The method 3520 can be practiced using any of the appliances or appliance sets described herein. In block 3522, a first orthodontic appliance is applied to a patient's teeth in order to reposition the teeth from a first tooth arrangement to a second tooth arrangement. In block 3524, a second orthodontic appliance is applied to the patient's teeth in order to reposition the teeth from the second tooth arrangement to a third tooth arrangement. The method 3520 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the next appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

FIG. 36 illustrates a method 3600 for designing an orthodontic appliance, in accordance with embodiments of the present technology. The method 3600 can be applied to any embodiment of the orthodontic appliances described herein. Some or all of the steps of the method 3600 can be performed by any suitable data processing system or device, e.g., one or more processors configured with suitable instructions.

In block 3602, a movement path to move one or more teeth from an initial arrangement to a target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, including measured or extrapolated hidden surfaces and root structures, as well as surrounding bone and soft tissue.

The target arrangement of the teeth (e.g., a desired and intended end result of orthodontic treatment) can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, a movement path can be defined for the motion of each tooth. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In block 3604, a force system to produce movement of the one or more teeth along the movement path is determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

Determination of the force system can be performed in a variety of ways. For example, in some embodiments, the force system is determined on a patient-by-patient basis, e.g., using patient-specific data. Alternatively or in combination, the force system can be determined based on a generalized model of tooth movement (e.g., based on experimentation, modeling, clinical data, etc.), such that patient-specific data is not necessarily used. In some embodiments, determination of a force system involves calculating specific force values to be applied to one or more teeth to produce a particular movement. Alternatively, determination of a force system can be performed at a high level without calculating specific force values for the teeth. For instance, block 3604 can involve determining a particular type of force to be applied (e.g., extrusive force, intrusive force, translational force, rotational force, tipping force, torquing force, etc.) without calculating the specific magnitude and/or direction of the force.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be measured, or input by a treating professional. In other embodiments, the treating professional can select an appropriate treatment based on physiological characteristics of the patient. For example, the properties of the palate may also be estimated based on factors such as the patient's age—for example, young juvenile patients can require lower forces to expand the suture than older patients, as the suture has not yet fully formed.

In block 3606, a design for an orthodontic appliance configured to produce the force system is determined. The design can include the appliance geometry, material composition and/or material properties, and can be determined in various ways, such as using a treatment or force application simulation environment. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systèmes of Waltham, MA.

Optionally, one or more designs can be selected for testing or force modeling. As noted above, a desired tooth movement, as well as a force system required or desired for eliciting the desired tooth movement, can be identified. Using the simulation environment, a candidate design can be analyzed or modeled for determination of an actual force system resulting from use of the candidate appliance. One or more modifications can optionally be made to a candidate appliance, and force modeling can be further analyzed as described, e.g., in order to iteratively determine an appliance design that produces the desired force system.

In block 3608, instructions for fabrication of the orthodontic appliance incorporating the design are generated. The instructions can be configured to control a fabrication system or device in order to produce the orthodontic appliance with the specified design. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by thermoforming.

Although the above steps show a method 3600 of designing an orthodontic appliance in accordance with some embodiments, a person of ordinary skill in the art will recognize some variations based on the teaching described herein. Some of the steps may comprise sub-steps. Some of the steps may be repeated as often as desired. One or more steps of the method 3600 may be performed with any suitable fabrication system or device, such as the embodiments described herein. Some of the steps may be optional, e.g., the process of block 3604 can be omitted, such that the orthodontic appliance is designed based on the desired tooth movements and/or determined tooth movement path, rather than based on a force system. Moreover, the order of the steps can be varied as desired.

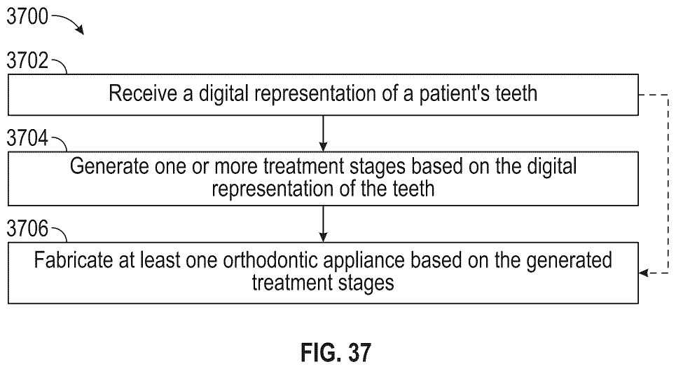

FIG. 37 illustrates a method 3700 for digitally planning an orthodontic treatment and/or design or fabrication of an appliance, in accordance with embodiments. The method 3700 can be applied to any of the treatment procedures described herein and can be performed by any suitable data processing system.

In block 3702 a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 3704, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 3706, at least one orthodontic appliance is fabricated based on the generated treatment stages. For example, a set of appliances can be fabricated, each shaped according to a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired.

In some instances, staging of various arrangements or treatment stages may not be necessary for design and/or fabrication of an appliance. As illustrated by the dashed line in FIG. 37, design and/or fabrication of an orthodontic appliance, and perhaps a particular orthodontic treatment, may include use of a representation of the patient's teeth (e.g., including receiving a digital representation of the patient's teeth (block 3702)), followed by design and/or fabrication of an orthodontic appliance based on a representation of the patient's teeth in the arrangement represented by the received representation.

The embodiments herein can be used in combination with aligners and/or a series of aligners with tooth-receiving cavities configured to move a person's teeth from an initial arrangement toward a target arrangement in accordance with a treatment plan. Aligners can include mandibular repositioning elements, such as those described in U.S. Pat. No. 10,912,629, entitled “Dental Appliances with Repositioning Jaw Elements,” filed Nov. 30, 2015; U.S. Pat. No. 10,537,406, entitled “Dental Appliances with Repositioning Jaw Elements,” filed Sep. 19, 2014; and U.S. Pat. No. 9,844,424, entitled “Dental Appliances with Repositioning Jaw Elements,” filed Feb. 21, 2014; all of which are incorporated by reference herein in their entirety.

As described above, the embodiments herein can be used in combination with dental auxiliary positioners, e.g., devices used to position prefabricated attachments and/or other auxiliaries on a person's teeth in accordance with one or more aspects of a treatment plan. Examples of dental auxiliary positioners (also known as “attachment placement devices,” “attachment placement templates” or “attachment fabrication templates”) can be found at least in: U.S. application Ser. No. 17/249,218, entitled “Flexible 3D Printed Orthodontic Device,” filed Feb. 24, 2021; U.S. application Ser. No. 16/366,686, entitled “Dental Attachment Placement Structure,” filed Mar. 27, 2019; U.S. application Ser. No. 15/674,662, entitled “Devices and Systems for Creation of Attachments,” filed Aug. 11, 2017; U.S. Pat. No. 11,103,330, entitled “Dental Attachment Placement Structure,” filed Jun. 14, 2017; U.S. application Ser. No. 14/963,527, entitled “Dental Attachment Placement Structure,” filed Dec. 9, 2015; U.S. application Ser. No. 14/939,246, entitled “Dental Attachment Placement Structure,” filed Nov. 12, 2015; U.S. application Ser. No. 14/939,252, entitled “Dental Attachment Formation Structures,” filed Nov. 12, 2015; and U.S. Pat. No. 9,700,385, entitled “Attachment Structure,” filed Aug. 22, 2014; all of which are incorporated by reference herein in their entirety.

The embodiments herein can be used in combination with incremental palatal expanders and/or a series of incremental palatal expanders used to expand a person's palate from an initial position toward a target position in accordance with one or more aspects of a treatment plan. Examples of incremental palatal expanders can be found at least in: U.S. application Ser. No. 16/380,801, entitled “Releasable Palatal Expanders,” filed Apr. 10, 2019; U.S. application Ser. No. 16/022,552, entitled “Devices, Systems, and Methods for Dental Arch Expansion,” filed Jun. 28, 2018; U.S. Pat. No. 11,045,283, entitled “Palatal Expander with Skeletal Anchorage Devices,” filed Jun. 8, 2018; U.S. application Ser. No. 15/831,159, entitled “Palatal Expanders and Methods of Expanding a Palate,” filed Dec. 4, 2017; U.S. Pat. No. 10,993,783, entitled “Methods and Apparatuses for Customizing a Rapid Palatal Expander,” filed Dec. 4, 2017; and U.S. Pat. No. 7,192,273, entitled “System and Method for Palatal Expansion,” filed Aug. 7, 2003; all of which are incorporated by reference herein in their entirety.

### EXAMPLES

The following examples are included to further describe some aspects of the present technology, and should not be used to limit the scope of the technology.

Example 1. A method for placing a dental auxiliary on a tooth, the method comprising:
providing a dental auxiliary positioner comprising an auxiliary to be mounted on a tooth of a patient, wherein the dental auxiliary positioner is provided in a first configuration corresponding to a first tooth arrangement of the patient's teeth;
determining a second tooth arrangement of the patient's teeth, wherein the second tooth arrangement corresponds to the patient's teeth at a time subsequent to the first tooth arrangement, and wherein a second position of the tooth in the second tooth arrangement is different from a first position of the tooth in the first tooth arrangement;
adjusting the dental auxiliary positioner to a second configuration to accommodate the difference between the first and second positions of the tooth; and
placing the auxiliary on the tooth using the adjusted dental auxiliary positioner.

Example 2. The method of Example 1, wherein the tooth is proximate to a space associated with a missing, unerupted, or erupting tooth.

Example 3. The method of Example 1 or 2, wherein the second arrangement of the patient's teeth is determined based on digital data of a current tooth arrangement of the patient's teeth.

Example 4. The method of Example 3, wherein the digital data is obtained using an intraoral scanner.

Example 5. The method of Example 3 or 4, further comprising:
comparing the digital data of the second tooth arrangement to digital data of the first tooth arrangement, and
identifying the difference between the first and second positions of the tooth based on the comparison.

Example 6. The method of Example 5, wherein the first configuration of the dental auxiliary positioner is based on the digital data of the first tooth arrangement.

Example 7. The method of any one of Examples 1 to 6, wherein the second tooth arrangement of the patient's teeth is measured using an adjustable device.

Example 8. The method of Example 7, wherein the adjustable device comprises an adjustable registration element configured to receive the tooth, and the method further comprises:
placing the adjustable device on the patient's teeth,
moving the adjustable registration element to accommodate the second position of the tooth, and
identifying the difference between the first and second positions of the tooth based on the movement of the adjustable registration element.

Example 9. The method of Example 8, wherein moving the adjustable registration element comprises one or more of translating or rotating the adjustable registration element.

Example 10. The method of Example 8 or 9, wherein the adjustable device comprises a plurality of measurement indicators, and wherein the adjustable registration element is moved relative to the measurement indicators to identify the difference between the first and second positions of the tooth.

Example 11. The method of any one of Examples 7 to 10, wherein the adjustable device is the dental auxiliary positioner.

Example 12. The method of any one of Examples 7 to 10, wherein the adjustable device is different from the dental auxiliary positioner.

Example 13. The method of any one of Examples 1 to 12, wherein:
when in the first configuration, the dental auxiliary positioner is configured to place the auxiliary at a predetermined location on the tooth in the first position, and
when in the second configuration, the dental auxiliary positioner is configured to place the auxiliary at the predetermined location on the tooth in the second position.

Example 14. The method of any one of Examples 1 to 13, wherein the dental auxiliary positioner comprises:
a registration element configured to receive the tooth, and
an auxiliary support coupled to the registration element and further coupled to the auxiliary.

Example 15. The method of Example 14, wherein the auxiliary support comprises:
a frame extending at least partially around the auxiliary,
one or more struts coupling the auxiliary to the frame, and
a bridge coupling the frame to the registration element.

Example 16. The method of Example 14 or 15, wherein adjusting the dental auxiliary positioner comprises adjusting the registration element, adjusting the auxiliary support, or a combination thereof.

Example 17. The method of any one of Examples 14 to 16, wherein the auxiliary support is movably coupled to the registration element, and adjusting the dental auxiliary positioner comprises moving the auxiliary support relative to the registration element.

Example 18. The method of any one of Examples 14 to 17, wherein the registration element is movably coupled to another portion of the dental auxiliary positioner, and adjusting the dental auxiliary positioner comprises moving the registration element relative to the portion of the dental auxiliary positioner.

Example 19. The method of any one of Examples 14 to 18, wherein the registration element is coupled to another portion of the dental auxiliary positioner, and adjusting the dental auxiliary positioner comprises separating the registration element from the portion of the dental auxiliary positioner.

Example 20. The method of any one of Examples 1 to 19, wherein the auxiliary comprises an attachment, a button, a power arm, a bracket, a splint, a distalizer, or a wire.

Example 21. The method of any one of Examples 1 to 20, wherein the auxiliary is configured to engage a dental appliance to apply a force to the tooth.

Example 22. A device for measuring tooth movement, the device comprising:
a base comprising a plurality of measurement indicators; and
one or more registration elements coupled to the base, wherein each registration element is configured to receive a tooth of a patient, and wherein the one or more registration elements comprise an adjustable registration element that is movable relative to the base to measure a difference between a first position and a second position of the corresponding received tooth.

Example 23. The device of Example 22, wherein the base comprises an elongate shaft that is configured to extend along at least a portion of the patient's arch.

Example 24. The device of Example 23, wherein the adjustable registration element is slidably coupled to the elongate shaft.

Example 25. The device of Example 23 or 24, wherein the adjustable registration element is rotatably coupled to the elongate shaft.

Example 26. The device of any one of Examples 22 to 25, wherein the adjustable registration element is movable relative to the base along one or more of a translational direction or a rotational direction.

Example 27. The device of any one of Examples 22 to 26, wherein the adjustable registration element is movable relative to the base along one or more of the following: a mesial-distal direction, a buccal-lingual direction, or a gingival-occlusal direction.

Example 28. The device of any one of Examples 22 to 27, wherein the adjustable registration element is configured to receive a tooth that is proximate to a space associated with a missing, unerupted, or erupting tooth.

Example 29. The device of any one of Examples 22 to 28, wherein the one or more registration elements comprise a plurality of registration elements.

Example 30. The device of Example 29, wherein the adjustable registration element is the only registration element that is movable relative to the base.

Example 31. The device of Example 29, wherein the plurality of registration elements comprise at least one additional adjustable registration element that is movable relative to the base.

Example 32. The device of any one of Examples 22 to 31, wherein the one or more registration elements each comprise a first portion coupled to the base, and a second portion configured to receive the corresponding tooth.

Example 33. The device of Example 32, wherein the second portion comprises an inner surface configured to engage a surface of the corresponding tooth.

Example 34. The device of Example 32 or 33, wherein the surface of the corresponding tooth comprises one or more of an occlusal surface, a buccal surface, or a lingual surface.

Example 35. The device of any one of Examples 22 to 34, wherein the adjustable registration element is further coupled to an auxiliary to be placed on the corresponding received tooth.

Example 36. The device of Example 35, wherein the auxiliary is coupled to the adjustable registration element via an auxiliary support comprising:
a frame extending at least partially around the auxiliary,
one or more struts coupling the auxiliary to the frame, and
a bridge coupling the frame to the adjustable registration element.

Example 37. The device of Example 35 or 36, wherein the auxiliary comprises an attachment, a button, a power arm, a bracket, a splint, a distalizer, or a wire.

Example 38. The device of any one of Examples 35 to 37, wherein the auxiliary is configured to engage a dental appliance to apply a force to the tooth.

Example 39. A system for placing a dental auxiliary on a tooth, the system comprising:
the device of any one of Examples 22 to 34; and
a dental auxiliary positioner comprising an auxiliary to be mounted on the tooth received by the adjustable registration element of the device, wherein the dental auxiliary positioner comprises a first configuration corresponding to a first tooth arrangement of the patient's teeth, and is adjustable to a second configuration to accommodate the second position of the received tooth.

Example 40. The system of Example 39, wherein:
when in the first configuration, the dental auxiliary positioner is configured to place the auxiliary at a predetermined location on the received tooth in the first position, and
when in the second configuration, the dental auxiliary positioner is configured to place the auxiliary at the predetermined location on the received tooth in the second position.

Example 41. The system of Example 39 or 40, wherein the dental auxiliary positioner comprises:
a registration element configured to receive the tooth, and an auxiliary support coupled to the registration element and further coupled to the auxiliary.

Example 42. The system of Example 41, wherein the auxiliary support comprises:
a frame extending at least partially around the auxiliary,
one or more struts coupling the auxiliary to the frame, and
a bridge coupling the frame to the registration element.

Example 43. The system of Example 41 or 42, wherein the auxiliary support is movably coupled to the registration element.

Example 44. The system of any one of Examples 41 to 43, wherein the registration element is movably coupled to another portion of the dental auxiliary positioner.

Example 45. The system of any one of Examples 41 to 44, wherein the registration element is releasably coupled to another portion of the dental auxiliary positioner.

Example 46. The system of any one of Examples 39 to 45, wherein the auxiliary comprises an attachment, a button, a power arm, a bracket, a splint, a distalizer, or a wire.

Example 47. The system of any one of Examples 39 to 46, wherein the auxiliary is configured to engage a dental appliance to apply a force to the tooth.

Example 48. A method for placing a dental auxiliary on a tooth, the method comprising:
obtaining, at a first time, data indicative of a tooth arrangement of a patient's teeth, wherein the patient's teeth comprise a tooth proximate to a space associated with a missing, unerupted, or erupting tooth;
positioning a dental spacing device into or proximate to the space proximate to the tooth, wherein the dental spacing device is configured to inhibit movement of the tooth relative to the space; and
placing, at a second time after the first time, an auxiliary on the tooth using a dental auxiliary positioner, wherein the dental auxiliary positioner has a configuration based on the data of the tooth arrangement of the patient's teeth at the first time.

Example 49. The method of Example 48, wherein the dental spacing device comprises:
a fixation portion configured to couple to the tooth, and
a spacer portion coupled to the fixation portion and configured to fit into or proximate to the space proximate to the tooth.

Example 50. The method of Example 49, further comprising adjusting a size of the spacer portion to fit into or proximate to the space proximate to the tooth.

Example 51. The method of Example 49 or 50, wherein the spacer portion comprises an adjustment mechanism.

Example 52. The method of any one of Examples 49 to 51, wherein the spacer portion comprises a deformable material.

Example 53. The method of any one of Examples 49 to 52, wherein the spacer portion is configured to obstruct movement of the tooth toward the space.

Example 54. The method of any one of Examples 49 to 53, wherein the fixation portion is configured to couple to one or more of a lingual surface, a buccal surface, or an interproximal surface of the tooth.

Example 55. The method of any one of Examples 49 to 54, wherein the fixation portion is configured to be coupled to the tooth via bonding, mechanical fixation, or a combination thereof.

Example 56. The method of any one of Examples 49 to 55, wherein the fixation portion is a first fixation portion, and the dental spacing device further comprises a second fixation portion coupled to the spacer portion and configured to engage another tooth proximate to the space.

Example 57. The method of any one of Examples 48 to 56, wherein the dental spacing device comprises an aesthetic portion configured to cover the space proximate to the tooth.

Example 58. The method of any one of Examples 48 to 57, wherein the second time is at least four weeks after the first time.

Example 59. The method of any one of Examples 48 to 58, further comprising removing the dental spacing device from the space immediately before placing the auxiliary on the tooth using the dental auxiliary positioner.

Example 60. The method of any one of Examples 48 to 59, wherein the dental auxiliary positioner is not adjustable.

Example 61. The method of any one of Examples 48 to 60, wherein the dental auxiliary positioner comprises:
a registration element configured to receive the tooth, and
an auxiliary support coupled to the registration element and further coupled to the auxiliary.

Example 62. The method of Example 61, wherein the auxiliary support comprises:
a frame extending at least partially around the auxiliary,
one or more struts coupling the auxiliary to the frame, and
a bridge coupling the frame to the registration element.

Example 63. The method of any one of Examples 48 to 62, wherein the auxiliary comprises an attachment, a button, a power arm, a bracket, a splint, a distalizer, or a wire.

Example 64. The method of any one of Examples 48 to 63, wherein the auxiliary is configured to engage a dental appliance to apply a force to the tooth.

Example 65. A device for maintaining a space in a patient's arch, the device comprising:
a fixation portion configured to couple to a tooth that is proximate to a space associated with a missing, unerupted, or erupting tooth;
a spacer portion coupled to the fixation portion and configured to fit into or proximate to the space proximate to the tooth to inhibit movement of the tooth toward the space; and
an aesthetic portion coupled to one or more of the fixation portion or the spacer portion, wherein the aesthetic portion is configured to cover the space proximate to the tooth and comprises a visual representation of one or more teeth.

Example 66. A system comprising:
the device of Example 65; and
a dental auxiliary positioner comprising an auxiliary to be mounted on the tooth proximate to the space.

Example 67. A device for bonding a dental auxiliary to a tooth, the device comprising:
a proximal portion and a distal portion, the distal portion comprising:
   one or more light output regions configured to radiate light having a wavelength configured to cure a light-curable dental compound; and
   one or more engager arms configured to engage one or more frames coupled to a dental auxiliary, wherein the one or more frames are part of a positioner assembly.

Example 68. The device of Example 67, further comprising a rotatable joint configured to enable the one or more engager arms to rotate after the one or more frames have been engaged so as to cause the one or more frames to break.

Example 69. The device of Example 67 or 68, further comprising an actuating control configured to actuate the one or more engager arms to engage the frame from the positioner assembly.

Example 70. The device of any one of Examples 67 to 69, further comprising an actuating control configured to extend the one or more engager arms distally to engage with the one or more frames.

Example 71. The device of any one of Examples 67 to 70, further comprising an actuating control configured to retract the one or more engager arms proximally to disengage the one or more frames from the positioner assembly.

Example 72. The device of any one of Examples 67 to 71, further comprising a dental auxiliary contact region configured to engage with the dental auxiliary.

Example 73. The device of Example 72, wherein the dental auxiliary contact region comprises a concave surface.

Example 74. The device of Example 72 or 73, further comprising a sensor configured to sense a pressure applied by the dental auxiliary contact region against the dental auxiliary seated within the dental auxiliary contact region.

Example 75. The device of any one of Examples 72 to 74, wherein the dental auxiliary contact region is configured to transmit the light radiated by the one or more light output regions.

Example 76. The device of any one of Examples 67 to 75, further comprising a handle at the proximal portion, the handle comprising an actuating control configured to actuate the one or more engager arms.

Example 77. The device of any one of Examples 67 to 76, further comprising a light control input configured to activate the one or more light output regions to radiate the light to cure the light-curable dental compound.

Example 78. The device of any one of Examples 67 to 77, wherein the one or more engager arms comprise one or more engagers at a distal end region of each of the one or more engager arms that is configured to engage with the one or more frames.

Example 79. A device for bonding a dental auxiliary to a tooth, the device comprising:
an actuation body having a proximal end and a distal end;
a tip region comprising one or more light output regions configured to radiate light having a wavelength that is configured to cure a light-curable dental compound;
a dental auxiliary contact region configured to engage with a dental auxiliary;
one or more engager arms at a distal end region of the tip region, the one or more engager arms configured to engage one or more frames coupled to the dental auxiliary, wherein the one or more frames are part of a positioner assembly; and
an actuating control configured to cause the one or more engager arms to engage with the one or more frames coupled to the dental auxiliary while the dental auxiliary is engaged with the dental auxiliary contact region.

Example 80. The device of Example 79, wherein the dental auxiliary contact region forms a part of the tip region.

Example 81. The device of Example 79 or 80, wherein the dental auxiliary contact region comprises a concave surface configured to conform to the dental auxiliary.

Example 82. The device of any one of Examples 79 to 81, further comprising a sensor configured to sense a pressure applied by the dental auxiliary contact region against the dental auxiliary seated within the dental auxiliary contact region.

Example 83. The device of Example 82, further comprising an output device configured to output a pressure indicator based on the sensed pressure applied by the dental auxiliary contact region against the dental auxiliary seated within the dental auxiliary contact region.

Example 84. The device of any one of Examples 79 to 83, wherein the dental auxiliary contact region is configured to transmit the light radiated by the one or more light output regions to the dental auxiliary.

Example 85. The device of any one of Examples 79 to 84, further comprising a rotatable joint configured to enable the one or more engager arms to rotate after the one or more frames have been engaged so as to cause the one or more frames to break.

Example 86. The device of any one of Examples 79 to 85, wherein the actuating control is configured to extend the one or more engager arms distally to engage the one or more frames.

Example 87. The device of any one of Examples 79 to 86, wherein the actuating control is configured to retract the one or more engager arms proximally to disengage the one or more frames from the positioner assembly.

Example 88. The device of any one of Examples 79 to 87, further comprising a light control input device configured to activate the one or more light output regions to radiate the light to cure the light-curable dental compound.

Example 89. The device of any one of Examples 79 to 88, further comprising a handle at the proximal end of the actuation body, wherein the handle includes the actuating control.

Example 90. The device of any one of Examples 79 to 89, wherein each engager arm comprises an engager at a distal end region of the engager arm that is configured to engage with the one or more frames.

Example 91. The device of any one of Examples 79 to 90, wherein the actuation body is configured to be manipulated by a hand of an operator.

Example 92. The device of any one of Examples 79 to 91, wherein the actuation body is configured to be controlled by a robotic system.

Example 93. An apparatus for adapting a light curing device for use with dental auxiliary, the apparatus comprising:
an adapter body configured to fit over the light curing device without optically occluding a light-emitting distal tip region of the light curing device; and
one or more engager arms at a distal end region of the adapter body configured to engage one or more frames coupled to a dental auxiliary, wherein the one or more frames are part of a positioner assembly.

Example 94. The apparatus of Example 93, further comprising a dental auxiliary contact region configured to engage with the dental auxiliary.

Example 95. The apparatus of Example 94, wherein the dental auxiliary contact region is configured to transmit light radiated by the light-emitting distal tip region of the light curing device.

Example 96. The apparatus of Example 94 or 95, wherein the dental auxiliary contact region comprises a concave surface.

Example 97. The apparatus of any one of Examples 94 to 96, further comprising a sensor configured to sense a pressure applied by the dental auxiliary contact region against the dental auxiliary seated within the dental auxiliary contact region.

Example 98. The apparatus of any one of Examples 93 to 97, further comprising an actuating control configured to extend the one or more engager arms to engage with the one or more frames.

Example 99. The apparatus of Example 98, wherein the actuating control is configured to extend the one or more engager arms distally to engage with the one or more frames.

Example 100. The apparatus of Example 98 or 99, wherein the actuating control is configured to retract the one or more engager arms proximally to disengage the one or more frames from the positioner assembly.

Example 101. The apparatus of any one of Examples 93 to 100, further comprising a rotatable joint configured to enable the one or more engager arms to rotate after the one or more frames have been engaged so as to cause the one or more frames to break.

Example 102. The apparatus of any one of Examples 93 to 101, further comprising a handle mount at a proximal end of the adapter body, the handle mount comprising an actuating control configured to actuate the one or more engager arms.

Example 103. An apparatus for removing a dental auxiliary from a tooth while reducing the release of debris, the apparatus comprising:
a first handle pivotally connected to a second handle;
a scraping jaw at a distal end region of the first handle;
an occlusal gripper at a distal end region of the second handle, wherein the occlusal gripper comprises a pocket configured to fit over the dental auxiliary; and
a suction lumen extending from a suction inlet at the distal end region of the first handle, at the distal end region of the second handle, or between the first and second handles, wherein the suction lumen is configured to aspirate debris as the dental auxiliary is being removed.

Example 104. The apparatus of Example 103, further comprising a suction connector at a proximal end region of the first or the second handle.

Example 105. The apparatus of Example 103 or 104, further comprising a suction source coupled to the suction lumen.

Example 106. The apparatus of Example 105, wherein the suction inlet is within the pocket, and wherein the suction lumen extends from the second handle to a suction connector at a proximal end region of the second handle.

Example 107. The apparatus of any one of Examples 103 to 107, wherein the suction inlet is adjacent to the scraping jaw at the distal end region of the first handle, and wherein the suction lumen extends from the first handle to a suction connector at a proximal end region of the first handle.

Example 108. The apparatus of any one of Examples 103 to 107, further comprising a reservoir configured to hold a putty material, wherein the putty material is configured to adhere to one or more of the dental auxiliary or fragments of the dental auxiliary.

Example 109. The apparatus of Example 108, further comprising a channel coupled to the reservoir, wherein the channel is configured to convey the putty material toward the distal end region of the first or second handle.

Example 110. The apparatus of any one of Examples 103 to 109, wherein the pocket comprises a concave surface configured to hold the dental auxiliary.

Example 111. The apparatus of any one of Examples 103 to 110, further comprising a shield extending around the distal end regions of the first handle and the second handle.

Example 112. The apparatus of Example 111, wherein the shield comprises a conical barrier.

Example 113. The apparatus of any one of Examples 103 to 112, further comprising a gripper shield extending around the occlusal gripper at the distal end region of the second handle.

Example 114. The apparatus of Example 113, wherein the gripper shield comprises a conical barrier.

Example 115. The apparatus of any one of Examples 103 to 114, wherein the scraping jaw comprises an edge configured to engage with the dental auxiliary or a bonding material coupling the dental auxiliary to a tooth.

Example 116. A method for bonding a dental auxiliary to a tooth, the method comprising:
emitting a light from a distal tip region of a light curing device to cure a light-curable dental compound on the dental auxiliary;
unlocking a release within the light curing device after the light has been emitted from the distal tip region for a cure time to permit one or more engager arms at a distal end region of the distal tip region to engage with a frame to which the dental auxiliary is attached; and
actuating the one or more engager arms to engage with the frame of the dental auxiliary to uncouple the frame from the dental auxiliary after the release has been unlocked.

Example 117. The method of Example 116, further comprising: sensing a pressure applied by the distal tip region of the light curing device against the dental auxiliary while emitting the light, and emitting an alert if the sensed pressure falls outside of a predetermined pressure range.

Example 118. The method of Example 117, wherein the release is unlocked after the light has been emitted from the distal tip region for the cure time while the sensed pressure is within the predetermined pressure range.

Example 119. The method of any one of Examples 116 to 118, further actuating the one or more engager arms comprises actuating an actuating control.

Example 120. The method of any one of Examples 116 to 119, wherein actuating the one or more engager arms comprises extending the one or more engager arms distally from the distal tip region to engage the frame.

Example 121. The method of any one of Examples 116 to 120, wherein actuating the one or more engager arms comprises retracting the one or more engager arms proximally while the one or more engager arms are engaged with the frame.

Example 122. The method of any one of Examples 116 to 121, wherein actuating the one or more engager arms comprises grabbing the frame with an engager at a distal end of each of the one or more engager arms.

Example 123. The method of any one of Examples 116 to 122, wherein actuating the one or more engager arms comprises rotating a rotatable joint to cause the frame to break.

Example 124. The method of any one of Examples 116 to 123, further comprising actuating suction from a suction port on the light curing device to remove debris from the frame.

Example 125. The method of any one of Examples 116 to 124, further comprising sensing an angle between the distal tip region and the dental auxiliary and triggering an alert if the angle is outside of a predetermined range.

Example 126. A method for bonding a dental auxiliary to a tooth, the method comprising:
sensing a contact or a pressure applied by a distal tip region of a light curing device against the dental auxiliary;
emitting a light from the distal tip region of the light curing device to cure a light-curable dental compound on the dental auxiliary when the sensed contact or pressure is within a predetermined range; and
emitting an alert if the sensed contact or pressure falls outside of the predetermined range.

Example 127. The method of Example 126, wherein the contact or pressure is sensed using one or more of a capacitive sensor, a piezoelectric sensor, or a mechanical sensor.

Example 128. The method of Example 126 or 127, wherein sensing the contact or pressure comprises deforming a spring element coupled to the distal tip region.

Example 129. The method of any one of Examples 126 to 128, wherein sensing the contact or pressure comprises sensing the contact.

Example 130. The method of any one of Examples 126 to 129, wherein sensing the contact or pressure comprises sensing the pressure.

Example 131. The method of any one of Examples 126 to 130, wherein the light is emitted while the sensed contact or pressure indicates sufficient contact between the distal tip region and the dental auxiliary.

Example 132. The method of any one of Examples 126 to 131, wherein the alert comprises one or more of a visual alert or an audible alert.

Example 133. The method of any one of Examples 126 to 132, wherein the alert is emitted from a region of the light curing device proximal to the distal tip region.

Example 134. The method of any one of Examples 126 to 133, further comprising unlocking a release within the light curing device after the light has been emitted from the distal tip region for the cure time, to permit one or more engager arms at a distal end region of the distal tip region to engage with a frame to which the dental auxiliary is attached.

Example 135. The method of Example 134, further comprising actuating the one or more engager arms to engage with the frame of the dental auxiliary to uncouple the frame from the dental auxiliary after the release has been unlocked.

Example 136. The method of Example 135, further comprising engaging the dental auxiliary with a pocket within the distal tip region of the light curing device.

Example 137. The method of Example 136, wherein sensing the contract or the pressure comprises sensing the contact or the pressure applied by the dental auxiliary to the pocket.

Example 138. The methods of any one of Examples 126 to 137, further comprising sensing an angle between the distal tip region and the dental auxiliary and triggering an alert if the angle is outside of a predetermined range.

Example 139. A method for bonding dental auxiliary to a tooth, the method comprising:
sensing a contact or a pressure applied by a distal tip region of a light curing device against the dental auxiliary;
emitting a light from the distal tip region of the light curing device to cure a light-curable dental compound on the dental auxiliary;
emitting an alert if the sensed contact or pressure is below a threshold;
unlocking a release of the light curing device after the light has been emitted from the distal tip region for a cure time to permit one or more engager arms at a distal end region of the distal tip region to engage with a frame of the dental auxiliary; and
actuating the one or more engager arms to engage with the frame of the dental auxiliary to uncouple the frame from the dental auxiliary after the release has been unlocked.

Example 140. A device for removing a dental auxiliary from a tooth, the device comprising:
a first elongate member comprising a proximal end and a distal end;
a second elongate member comprising a proximal end and a distal end, wherein the first and second elongate members are pivotally coupled to each other;
a scraper at the distal end of the second elongate member, wherein the scraper is configured to apply force to the dental auxiliary to remove the dental auxiliary from the tooth; and
a pair of sidewalls configured to capture debris resulting from the removal of the dental auxiliary from the tooth.

Example 141. The device of Example 140, wherein at least a portion of the scraper is a hollow structure.

Example 142. The device of Example 140, wherein the scraper is a solid structure.

Example 143. The device of any one of Examples 140 to 142, wherein the scraper comprises a lower surface configured to contact the dental auxiliary.

Example 144. The device of Example 143, wherein the lower surface is configured to direct the debris between the pair of the sidewalls.

Example 145. The device of Example 143, wherein the lower surface is angled or concave.

Example 146. The device of any one of Examples 140 to 145, wherein the scraper comprises a distal surface that is angled in a proximal direction.

Example 147. The device of any one of Examples 140 to 146, further comprising a platform at the distal end of the first elongate member, wherein the platform is configured to support the tooth.

Example 148. The device of Example 147, wherein the sidewalls are located proximal to the platform.

Example 149. The device of Example 147 or 148, wherein the platform is integrally formed with the first elongate member.

Example 150. The device of Example 147 or 148, wherein the platform is a discrete component that is coupled to the first elongate member.

Example 151. The device of Example 150, wherein the first elongate member is composed of a first material and the platform is composed of a second material, wherein the first and second materials are different materials, and wherein the first and second material have different rigidities.

Example 152. The device of any one of Examples 147 to 151, further comprising a rotatable assembly pivotally coupled to the distal ends of the first and second elongate members, wherein the scraper and the platform are located on the rotatable assembly.

Example 153. The device of Example 152, wherein the rotatable assembly is configured to rotate to a plurality of different orientations relative to the first and second elongate members.

Example 154. The device of Example 153, further comprising a control configured to control the rotation of the rotatable assembly.

Example 155. The device of Example 154, wherein the control is located on at least one of the proximal ends of the first and second elongate members.

Example 156. The device of any one of Examples 152 to 155, further comprising a locking feature configured to lock the rotatable assembly at a fixed orientation relative to the distal ends of the first and second elongate members.

Example 157. The device of any one of Examples 140 to 156, wherein the pair of sidewalls are integrally formed with the first elongate member.

Example 158. The device of any one of Examples 140 to 156, wherein the pair of sidewalls are located on a removable sleeve that is configured to be coupled to the device.

Example 159. The device of any one of Examples 140 to 158, wherein the distal ends of the first and second elongate members are curved or angled.

Example 160. The device of any one of Examples 140 to 159, wherein the proximal ends of the first and second elongate members are configured to be handled by a user to open and close the distal ends of the first and second elongate members.

Example 161. The device of Example 160, wherein the closing of the distal ends of the first and second elongate members causes the scraper to apply the force to the dental auxiliary to remove the dental auxiliary from the tooth.

Example 162. The device of any one of Examples 140 to 161, wherein the debris comprises the dental auxiliary or a fragment thereof, a bonding material coupling the dental auxiliary to the tooth, or a combination thereof.

Example 163. The device of any one of Examples 140 to 162, further comprising a shield at least partially surrounding at least one of the distal ends of the first and second elongate members, wherein the shield is configured to capture the debris resulting from the removal of the dental auxiliary from the tooth.

Example 164. The device of any one of Examples 140 to 163, further comprising a suction inlet, wherein the suction inlet is configured to aspirate the debris resulting from the removal of the dental auxiliary from the tooth.

Example 165. The device of any one of Examples 140 to 164, further comprising an energy output region, wherein the energy output region is configured to output energy to cure a bonding material for coupling a second dental auxiliary to a tooth.

Example 166. The device of Example 165, further comprising one or more engager arms proximate to the energy output region, wherein the one or more engager arms are configured to engage with a frame of the second dental auxiliary.

Example 167. The device of Example 165 or 166, further comprising a dental auxiliary contact region configured to contact the second dental auxiliary.

Example 168. The device of Example 167, further comprising a sensor configured to monitor one or more of contact, pressure, or an angle of the dental auxiliary contact region against the second dental auxiliary.

Example 169. A method for removing a dental auxiliary from a tooth, the method comprising:
positioning a first distal end of a dental auxiliary removal device proximate to a dental auxiliary on a tooth of a patient, wherein the first distal end comprises or is coupled to a scraper;
positioning a second distal end of the dental auxiliary removal device proximate to the tooth, wherein the second distal end comprises or is coupled to a platform;
removing the dental auxiliary from the tooth by applying a force against the dental auxiliary using the scraper while the tooth is supported by the platform; and
directing debris generated by the removal of the dental auxiliary between a pair of sidewalls proximate to the platform at the second distal end of the dental auxiliary removal device.

Example 170. The method of Example 169, wherein the scraper and the platform are located on a rotatable assembly pivotally coupled to the first and second distal ends, and wherein the method further comprises rotating the rotatable assembly to a target orientation relative to the first and second distal ends.

Example 171. The method of Example 169 or 170, wherein the sidewalls are part of the dental auxiliary removal device.

Example 172. The method of Example 169 or 170, further comprising coupling a sleeve to the dental auxiliary removal device, wherein the sidewalls are part of the sleeve.

### CONCLUSION

Although many of the embodiments are described above with respect to systems, devices, and methods for placement of dental attachments on teeth and/or removal of dental attachments from teeth, the present technology is applicable to placement and/or removal of other types of dental auxiliaries. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-37.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

What is claimed is:

1. A device for removing a dental auxiliary from a tooth, the device comprising:
a first elongate member comprising a proximal end and a distal end;
a second elongate member comprising a proximal end and a distal end, wherein the first and second elongate members are pivotally coupled to each other;
a scraper at the distal end of the second elongate member, wherein the scraper is configured to apply force to the dental auxiliary to remove the dental auxiliary from the tooth; and
a pair of sidewalls configured to capture debris resulting from the removal of the dental auxiliary from the tooth.

2. The device of claim 1, wherein at least a portion of the scraper is a hollow structure.

3. The device of claim 1, wherein the scraper comprises a lower surface configured to contact the dental auxiliary, and wherein the lower surface is configured to direct the debris between the pair of the sidewalls.

4. The device of claim 1, wherein the lower surface is angled or concave, and wherein the scraper comprises a distal surface that is angled in a proximal direction.

5. The device of claim 1, further comprising a platform at the distal end of the first elongate member, wherein the platform is configured to support the tooth.

6. The device of claim 5, wherein the sidewalls are located proximal to the platform.

7. The device of claim 5, wherein the platform is a discrete component that is coupled to the first elongate member.

8. The device of claim 5, wherein the first elongate member is composed of a first material and the platform is composed of a second material, wherein the first and second materials are different materials, and wherein the first and second material have different rigidities.

9. The device of claim 5, further comprising a rotatable assembly pivotally coupled to the distal ends of the first and second elongate members, wherein the scraper and the platform are located on the rotatable assembly, and wherein the rotatable assembly is configured to rotate to a plurality of different orientations relative to the first and second elongate members.

10. The device of claim 9, further comprising a control configured to control the rotation of the rotatable assembly.

11. The device of claim 10, wherein the control is located on at least one of the proximal ends of the first and second elongate members.

12. The device of claim 9, further comprising a locking feature configured to lock the rotatable assembly at a fixed orientation relative to the distal ends of the first and second elongate members.

13. The device of claim 1, wherein the pair of sidewalls are integrally formed with the first elongate member.

14. The device of claim 1, wherein the pair of sidewalls are located on a removable sleeve that is configured to be coupled to the device.

15. The device of claim 1, wherein the distal ends of the first and second elongate members are curved or angled.

16. The device of claim 1, wherein the proximal ends of the first and second elongate members are configured to be handled by a user to open and close the distal ends of the first and second elongate members, and wherein the closing of the distal ends of the first and second elongate members causes the scraper to apply the force to the dental auxiliary to remove the dental auxiliary from the tooth.

17. A method for removing a dental auxiliary from a tooth, the method comprising:
positioning a first distal end of a dental auxiliary removal device proximate to a dental auxiliary on a tooth of a patient, wherein the first distal end comprises or is coupled to a scraper;
positioning a second distal end of the dental auxiliary removal device proximate to the tooth, wherein the second distal end comprises or is coupled to a platform;
removing the dental auxiliary from the tooth by applying a force against the dental auxiliary using the scraper while the tooth is supported by the platform; and
directing debris generated by the removal of the dental auxiliary between a pair of sidewalls proximate to the platform at the second distal end of the dental auxiliary removal device.

18. The method of claim 17, wherein the scraper and the platform are located on a rotatable assembly pivotally coupled to the first and second distal ends, and wherein the method further comprises rotating the rotatable assembly to a target orientation relative to the first and second distal ends.

19. The method of claim 17, wherein the sidewalls are part of the dental auxiliary removal device.

20. The method of claim 17, further comprising coupling a sleeve to the dental auxiliary removal device, wherein the sidewalls are part of the sleeve.
